(19) United States
(12) Patent Application Publication
(10) Pub. No.: US 2024/0189797 A1
(43) Pub. Date: Jun. 13, 2024

(54) SEPARATION MEDIA AND PURIFICATION METHODS FOR CARBOHYDRATE BINDING DOMAIN CONTAINING MOLECULES

(71) Applicant: Donaldson Company, Inc., Bloomington, MN (US)

(72) Inventors: Jinxiang Zhou, Greenville, SC (US); Graham Temples, Greenville, SC (US); Daniel Henn, Wilmington, DE (US)

(21) Appl. No.: 18/383,453

(22) Filed: Oct. 24, 2023

Related U.S. Application Data
(60) Provisional application No. 63/419,204, filed on Oct. 25, 2022.

(51) Int. Cl. B01J 20/32; B01D 15/38; B01J 20/22; B01J 20/24; B01J 20/289; C07K 1/22; C07K 14/005

(52) U.S. Cl. CPC B01J 20/3265; B01D 15/3828; B01J 20/223; B01J 20/24; B01J 20/289; B01J 20/3206; C07K 1/22; C07K 14/005; B01D 2221/10; B01J 2220/44; B01J 2220/52

(57) ABSTRACT

Separation media includes a support substrate and a plurality of separation ligands immobilized on the support substrate. The plurality of separation ligands include an affinity capable of recognizing and binding to a carbohydrate recognizing domain. Methods of making the separation media and methods of using the separation media are disclosed.

### CROSS-REFERENCE TO RELATED APPLICATION

This application claims the benefit of U.S. Provisional Patent Application No. 63/419,204, filed Oct. 25, 2022, which is incorporated herein by reference in its entirety.

### GOVERNMENT FUNDING

This invention was made with government support under AI149987 awarded by the National Institutes of Health. The government has certain rights in the invention.

### FIELD

The present disclosure relates to separation media and separation devices containing the same. The separation media of the present disclosure may be useful for isolation and/or concentration of biomolecules (e.g., viruses, proteins or fragments thereof) that include a carbohydrate binding domain. The separation media of the present disclosure may be used for separations in membrane chromatography. The present disclosure further relates to methods of making and using the separation media.

### INTRODUCTION

Each year, from 5 to 20 percent of the US population becomes ill with influenza. More than 200,000 are hospitalized, and about 36,000 individuals die. Worldwide, there are roughly 3-5 million yearly cases of severe flu illness and up to 500,000 deaths as a result. Vaccine development and production time is key to combat the emergence of new pandemic influenza strains. Recombinant hemagglutinin (rHA) vaccines show great promise for a rapid and scalable pandemic response. However, the slow and low-yielding purification steps required to obtain sufficient vaccine purity is a critical barrier for the commercial-scale implementation of recombinant vaccine manufacturing.

### Summary

This disclosure describes, in one aspect, a separation media that includes a support substrate and a plurality of separation ligands immobilized on the support substrate. The plurality of separation ligands are of the formula SL:

-L-Z (SL)
where L is a linker and Z is a separation group. The separation group includes an affinity group. The affinity group includes a carbohydrate or a small molecule. The separation media may be configured for isolating a target molecule comprising a carbohydrate binding domain. In some embodiments, the target molecule includes hemagglutinin. In some embodiments, the target molecule includes a recombinant hemagglutinin based vaccine.

In some embodiments, the formula SL is of formula SL1 or SL2

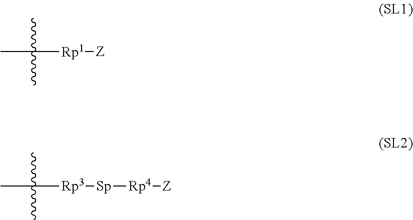

In formula SL1 and SL2 Rp¹, Rp³, and Rp⁴ each independently comprise the reaction product of any one of Rp^{A}, Rp^{B}, Rp^{C}, Rp^{D}, Rp^{E}, Rp^{F}, Rp^{G}, Rp^{H}, Rp^{I}, Rp^{J}, Rp^{K}, or an isomer thereof

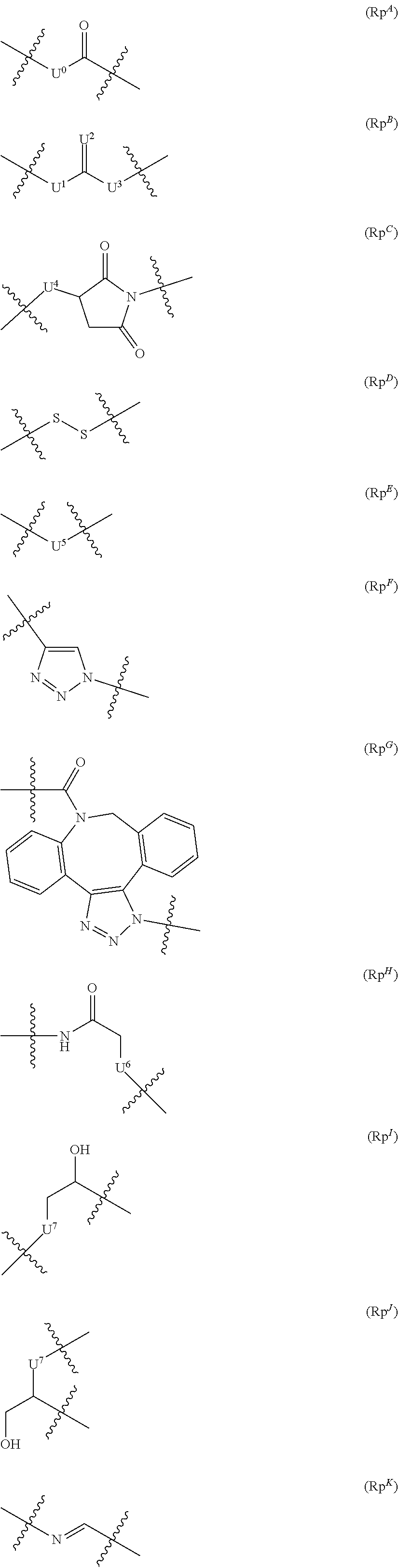

where U⁰, U¹, U², U³, U⁴, U⁵, U⁶, and U⁷ are each independently NH, N, O, or S and Sp is a spacer.

In some embodiments, the separation ligand formula SL or SL1 is of formula X, XI, XII, or XIII:

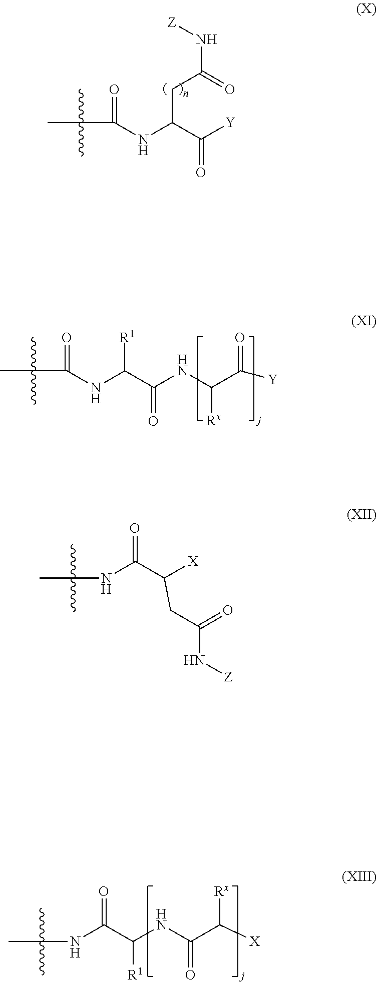

where n is 0, 1, 2, 3, or 4; X is NH₂ or PG_{N} where PG_{N} is an amine protecting group; Y is OH or a PG_{C(O)OH} where PG_{C(O)CH} is a carboxylic acid protecting group; R¹ is an amino acid side chain; j is 1, 2, 3, 4, 5, 6, 7, 8, 9, or 10; each R^{X} is independently an amino acid side chain or

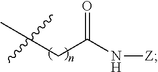

and at least one R^{X} is

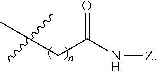

In another aspect, this disclosure describes a separation device that includes a housing and a separation media of the present disclosure disposed within the housing.

In another aspect, this disclosure describes a method for isolating a target molecule from an isolation solution. The isolation solution includes an isolation solvent and the target molecule. The target molecule includes a carbohydrate recognizing domain. The method includes contacting the isolation solution with the separation media or separation device of the present disclosure.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### Definitions

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

As used here, the symbol “

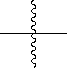

” (referred to here as “a point of attachment bond”) denotes a bond that is a point of attachment between two chemical entities, or a chemical entity and a support substrate, one of which is depicted as being attached to the point of attachment bond and the other of which is not depicted. For example, “

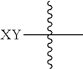

” indicates that the chemical entity “XY” is bonded to another chemical entity or support substrate via the point of attachment bond.

The term “organic group” refers to a group that has carbon-hydrogen bonds. The group may also include heteroatoms such as O, S, N, or P. One or more heteroatoms may be catenated at any location in the organic group (e.g., ether, thioether, or amine). A heteroatom may be covalently bonded to a carbon atom through a double bond (e.g., ketone, imine). A heteroatom covalently bonded to a carbon atom may also be covalently bonded to another heteroatom (e.g., phosphodiester, sulfone). One or more functional groups may be included in an organic group, for example, alkane (branched, linear, or cyclic), alkene (branched or linear), alkyne (branched or linear), aromatic, amine (primary, secondary, tertiary, or quaternary), amino, amide, alcohol (primary, secondary, or tertiary), alkoxy, aldehyde, carboxylic acid, ether, ester, imine, phosphoester, phosphodiester, sulfone, sulfonamide, urea, thiourea, thioether, any combination thereof, and ionized versions thereof. Generally, the organic group may be covalently bonded to a compound. The point of attachment of the organic group to the compound may be described in several ways. For example, in some embodiments, the organic group may be described as the monovalent or radical of the respective functional group (e.g., alkyl for alkane, aryl for aromatic ring, aminyl for a primary or secondary amine). In some embodiments, where a general formula is shown with a covalent bond connecting the organic group to a compound, the organic group may be described as the common functional group. For example, if the organic group R is described relative to the formula CH₃CH₂CH₂—R, the organic group may be described, for example, as an aromatic ring.

The term “catenated” in the context of heteroatoms refers to a heteroatom (e.g., O, S, N, P) that replaces at least one carbon atom in a carbon chain. For example, ether groups contain one catenary oxygen atom with at least one carbon atom on each side of the catenary oxygen atom and polyether groups contain more than one catenary oxygen atom with carbon atoms on each side of the more than one catenary oxygen atoms.

The term “aryl” refers to a monovalent group that is aromatic. The aryl group may be carbocyclic or include one or more heteroatoms such as S, N, or O. Example aryl groups include, but are not limited to, phenyl, thiophenyl, furanyl, pyridinyl, pyrimidinyl, piperidinyl, and pyrrolyl.

The term “alkylene” or “alkanediyl” refers to a divalent group that is a radical of an alkane and includes groups that are linear, branched, cyclic, bicyclic, or a combination thereof.

Unless otherwise indicated, the alkylene group typically has 1 to 30 carbon atoms. In some embodiments, the alkylene group has 1 to 20 carbon atoms, 1 to 10 carbon atoms, 1 to 6 carbon atoms, or 1 to 4 carbon atoms. Examples of “alkylene” groups include methylene, ethylene, propylene, 1,4-butylene, 1,4-cyclohexylene, and 1,4-cyclohexyldimethylene.

“Alkenyl” or “alkenyl group” refers to a straight or branched hydrocarbon chain radical having from two to forty carbon atoms, and having one or more carbon-carbon double bonds. Each alkenyl group is attached to the rest of the molecule by a single bond. Unless stated otherwise specifically in the specification, an alkyl group can be optionally substituted.

“Alkoxy” refers to the group —OR, where R is alkyl, alkenyl, alkynyl, cycloalkyl, or heterocycle as defined herein. Unless stated otherwise specifically in the specification, alkoxy can be optionally substituted.

The term “backbone” refers to the longest contiguous chain. One or more branches may be covalently bonded to the backbone.

The term “aromatic” refers to a cyclic, fully conjugated planar structure that obeys Hückel's rules, that is the compound has 4n+2π electrons where n is a positive integer or zero. For example, benzene has 6π electrons. Thus, 6=4n+2π. Solving for n gives 1. Therefore, benzene is an aromatic compound.

The term “kosmotrope” is generally used to denote a solute that increases the degree of ordered-ness of water by stabilizing water-water interactions. Kosmotropes may be ionic or non-ionic. In contrast, the term “chaotrope” is generally used to denote a solute that decreases the degree of ordered-ness of water by destabilizing water-water interactions. Chaotropes may be ionic or non-ionic.

The term “peptide” refers to a sequence of amino acid residues without regard to the length of the sequence. Therefore, the term “peptide” refers to any amino acid sequence having at least two amino acids and includes full-length proteins and, as the case may be, polyproteins.

The term “polypeptide” refers to a sequence of amino acid residues without regard to the length of the sequence. Therefore, the term “polypeptide” refers to any amino acid sequence having at least two amino acids and includes full-length proteins, fragments thereof, and/or, as the case may be, polyproteins.

The term “protein” refers to any sequence of two or more amino acid residues without regard to the length of the sequence, as well as any complex of two or more separately translated amino acid sequences. Protein also refers to amino acid sequences chemically modified to include a carbohydrate, a lipid, a nucleotide sequence, or any combination of carbohydrates, lipids, and/or nucleotide sequences. As used herein, “protein,” “peptide,” and “polypeptide” are used interchangeably.

In the description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be performed in any feasible order. And, as appropriate, any combination of two or more steps may be performed simultaneously.

### BRIEF DESCRIPTION OF FIGURES

FIG. 1 shows the structure of neuraminic acid.

FIG. 2A shows the structure of an α(2,3) N-acetylneuraminic acid-galactose motif.

FIG. 2B shows the structure of α(2,6) N-acetylneuraminic acid-galactose motif.

FIG. 3A shows a schematic of a linear and branched glycan structure.

FIG. 3B shows a schematic of a branched glycan structure having two terminal N-acetylneuraminic acid-galactose motifs.

FIG. 4A is a flow diagram of a first method for making the separation media of the present disclosure.

FIG. 4B is a flow diagram of a second method for making the separation media of the present disclosure.

FIG. 5A is a flow diagram of a third method for making the separation media of the present disclosure.

FIG. 5B is a flow diagram of a fourth method for making the separation media of the present disclosure.

FIG. 6A is a flow diagram of a fifth method for making the separation media of the present disclosure.

FIG. 6B is a flow diagram of a sixth method for making the separation media of the present disclosure.

FIG. 7A is a schematic of a separation media consistent with embodiments of the present disclosure.

FIG. 7B is a schematic representation of a separation device consistent with embodiments of the preset disclosure.

FIG. 8 is a flow diagram of a method of using the separation media and/or separation devices of the present disclosure.

FIG. 9 is a schematic of a first synthetic strategy for making the separation media consistent with embodiments of the present disclosure. This method includes the indirect immobilization of separation ligands through the grafting of a polymer from the support substrate. This method also includes the amine assisted method.

FIG. 10A is a plot showing the degree of grafting (%) of various polymers made from different monomers at different initiation times.

FIG. 10B is a plot showing the degree of grafting (%) of a poly(HEA) when different amounts of the HEA monomer were used during polymerization.

FIG. 11A is a plot showing the relationship of initiator concentration and degree of grafting on 0.45 μm pore size membrane.

FIG. 11B is a plot showing the relationship between degree of grafting and the percent mass change when 10 mg DSC/mL DMSO was used in step 3 of FIG. 9.

FIG. 12 is schematic of a second synthetic strategy for making the separation media of consistent with embodiments of the present disclosure. This method includes the direct immobilization of separation ligands using the amine assisted method.

FIG. 13 is a schematic of a third synthetic strategy for making the separation media consistent with embodiments of the present disclosure. This method includes the direct immobilization of separation ligands using the organic solvent assisted method.

FIG. 14 is and SDS-PAGE gel comparing non-reducing and reducing conditions of elution pools or recombinant HA (rHA) purified protein using: the separation media column using method (M1) 1; the separation media column using method 2 (M2); or a Cyvita CAPTO IMPACT column (S) (M-Ladder).

FIG. 15. shows a synthetic scheme for the synthesis of aminated 6′sialyllactose from 6′sialyllactose.

### DETAILED DESCRIPTION

The present disclosure provides separation media and separation devices containing the same. Specifically, the disclosure provides separation media that may be used to concentrate or separate (e.g., purify) a target molecule that includes a carbohydrate binding domain. To that end, the separation media of the present disclosure includes separation ligands. The separation ligands include a separating group that can be an affinity group, an assistance group, or a capping group. The affinity group includes a carbohydrate to which the target molecule can bind. Multiple separation media of the present disclosure may be arranged in a stacked configuration to increase separation specificity and/or efficiency. The separation media of the present disclosure may be used for separations in membrane chromatography.

A carbohydrate is a monosaccharide, a disaccharide, or a polysaccharide (also called a glycan). Glycans may include a variety of saccharides of varying identities arranged in a glycosylation pattern; that is, the pattern of saccharides in a glycan that includes the identity of the saccharides and the configuration of the connection between adjacent saccharides. Glycans can be conjugated to various bio-molecules to create glyco-biomolecules (i.e., biomolecules that include a glycan group). For example, glycans may be conjugated to lipids, capsids (the protein shell of a virus), and proteins to create glycolipids, glycoproteins, and glycocapsids respectively. A biomolecule that includes a glycan group may be referred to as having been glycosylated (e.g., a glycosylated protein). The binding of various biomolecules to specific glycosylation patterns of glycosylated biomolecules regulate many biological processes.

Glyco-biomolecules and biomolecules that bind thereto play a role in various cellular processes in both healthy and disease states. For example, cancer cells may have altered glycosylation patterns that contribute to tumor growth. Additionally, some viruses have surface proteins that can bind to various glycosylated biomolecules displayed extracellularly on human cells. Some viruses bind to specific glycosylation patters. For example, some viruses bind to specific terminal sialic acid-galactose glycosylation motifs (e.g., N-acetylneuraminic acid-galactose motif). Upon binding to such a motif, the virus may gain entrance to infect the cell. Additionally, various proteins can bind to carbohydrates. For example, lectins can bind to specific glycosylation patterns displayed on cell surfaces to evoke downstream response to regulate, for example, cell adhesion, glycoprotein synthesis, and the immune system.

Because of their ability to target cells displaying specific glycosylation patterns, modified viruses and lectins are being explored for use as therapeutics and diagnostic tools. For example, viruses are being explored for use as viral vectors to deliver gene therapy reagents to cells and are used to deliver vaccines. Lectins are being investigated for use as an HIV therapy, anti-cancer therapy, cancer diagnostics, and as targeting agents for other therapeutics. To facilitate research and applications of glycan recognizing biomolecules, improved purification media and techniques are needed to isolate and/or concentrate such biomolecules.

Hemagglutinin (HA) vaccines, such as recombinant HA vaccines, are being explored as vaccines for the prevention of various virial infections including influenza. HAs are homotrimeric surface glycoproteins produced by some viruses that causes red cells to clump together, or agglutinate. HA proteins include sialic acid and use the sialic acid to bind to receptors on the surface of red blood cells. HAs are the primary antigen responsible for immune response in various viruses such as influenza. Different viruses produce and display different HA proteins. For example, the influenza A virus has at least 18 subtypes of hemagglutinin (H1-H18). Other examples of HA proteins include measles hemagglutinin, parainfluenza hemagglutinin-neuraminidase, mumps hemagglutinin-neuraminidase, influenza type B hemagglutinin, influenza type C hemagglutinin-esterase fusion, influenza type D hemagglutinin-esterase fusion, and phytohemagglutinin.

Currently, the majority of influenza vaccines are produced by growing the seasonally matched virus strains in chicken eggs. However, the decades-old egg-based platform is not suited to respond to the emergence of a pandemic strain due its lengthy development time (several months) and limited scalability. The scalability issue is largely overcome by the mammalian cell culture-based approach for propagation of the vaccine virus, but long development time and uncertainties involved in adapting the virus for the growth medium is still a major challenge. For these reasons, there is a desire to shift vaccine production to a recombinant protein (recombinant HA (rHA)) manufacturing process due to its quick startup time, flexibility, and scalability. Recombinant proteins are proteins made using recombinant DNA technology. Recombinant proteins are made by introducing recombinant DNA that encodes a protein (such as a hemagglutinin protein) into a host, expressing the protein, and purifying the protein from the cell. The host may be a bacterial cell, insect cell, or mammalian cell. Recombinant vaccines are vaccines that include a recombinant protein. For example, a hemagglutinin vaccine may be a recombinant hemagglutinin vaccine.

Conventional methods for vaccine purification fail to meet the growing needs for high purity recombinant HA influenza vaccines. Density gradient centrifugation is the traditional method for purification of egg-based influenza virus vaccines but is not suitable for purification of the relatively small recombinant protein vaccines. Resin chromatography has been used broadly for protein purification due to its scalability and consistency. However, resins require long processing time due to slow mass transfer of proteins through the small pores. In contrast, macroporous membrane chromatography has been proven to provide at least ten times faster processing speed than traditional resins. The much larger pores of membranes allow for high binding capacity for proteins and viruses with short residence time (<6 s). Ion-exchange chromatography columns, as part of a tedious multistep process, have been shown to be effective to achieve high purity vaccines. These processes, which may involve multiple chromatography steps and up to nine total steps, could be dramatically simplified through the use of affinity chromatography, due to its high selectivity. So called “affinity-like” membrane and resin technologies based on ligands such as sulfonated cellulose, and dextran sulfate have shown improvement in some cases over ion-exchange technologies in terms of loading capacity and selectivity for vaccine purification. However, no commercial affinity membrane chromatography columns are available that have the ability to discriminate between the biologically active HA vaccine species and the related inactive or denatured forms.

Traditionally, downstream purification of biomolecules has been expensive, slow, and difficult to scale. Typical biomolecule purification trains include various steps such as centrifugation, filtering, and one or more chromatography separations using one or more types of chromatography columns (e.g., size exclusion columns and affinity chromatography columns). A typical chromatography column used in biomolecule purification may include a packed bed column with resin configured for size exclusion chromatography, reverse phase chromatography, or affinity chromatography. Resin based chromatography columns have been the gold standard employed to purify biologics for decades. However, column chromatography in large volumes may be very slow. Additionally, resin columns are known to require long residence times to perform adequately.

Compared to resins, macroporous membranes can achieve over an order of magnitude faster processing speeds. The rapid purification afforded by the large flow-through pores historically has come at the cost of lower binding capacity due to decreased specific surface area compared to resins. Strategies to increase the surface area of membrane supports have included electrospun nanofiber membrane fabrication methods and hydrogel membranes. Unfortunately, these membranes are either difficult to scale up, have poor mechanical properties, or have small mesh size resulting in poor macromolecule accessibility, loss of binding capacity at short residence times, and high pressure drops. Efforts to increase surface area and ligand density can have negative impacts on binding capacity due to steric hindrance from ligand crowding.

The present disclosure describes separation media that may be used for separation in membrane chromatography. In contrast to resin columns, membrane adsorbers perform well at short column residence times, potentially providing rapid separations for biologics. The present disclosure provides separation media that are suitable for separation, purification, and/or concentration of a target that include a carbohydrate recognizing domain. The term “carbohydrate recognizing domain” refers to a biomolecule, such as a protein, or a portion thereof, that is capable of recognizing a carbohydrate. The term “carbohydrate” refers to a monosaccharide, disaccharide, or glycan.

Molecules of interest that may be separated using the separation media of the present disclosure are collectively referred to here as target molecules or as targets. The target molecules may be present in a solution, suspension, or dispersion. For simplicity, the liquid containing the target molecule is referred to here as an isolation solution. Also, for simplicity, a target may be referred to in the singular but it is understood that an isolation solution may include a plurality of target molecules of the same identity. An isolation solution may also include two or more targets of different identity. The isolation solution may be or include the media or lysate of a recombinant or natural expression system used to make the target molecule. As such, the isolation solution may include other biomolecules or cellular debris. The separation media may be configured for concentrating the target from an isolation solution of already purified target. As such, the isolation solution may include the target molecule, one or more buffering agents, and one or more salts. The isolation solution containing the target molecule may also include solvents, such as water, an organic solvent, or a combination thereof, and soluble components dissolved in the solvent. The separation media may be configured for use with an organic solvent. The separation media may be configured to separate or purify the target molecules from an isolation solution that includes an organic solvent.

In some embodiments, the target includes a carbohydrate binding domain. In some embodiments, the carbohydrate binding domain is a part of a protein. In some embodiments, the carbohydrate binding domain is a part of a hemagglutinin protein; that is, the target includes a hemagglutinin protein or a fragment thereof. In some embodiments, the hemagglutinin is recombinant hemagglutinin (rHA). In some embodiments, the hemagglutinin is expressed in animals such as, for example, chicken eggs. In some embodiments, the hemagglutinin is expressed in insects or insect cells. In some embodiments, the target includes a HA vaccine. In some embodiments, the target includes a recombinant HA vaccine. In some embodiments, the carbohydrate binding domain can bind to sialic acid.

In some embodiments, the target includes a virus, modified virus, or viral vector. In some such embodiments, the viral vector includes hemagglutinin. In some embodiments, the target includes an influenza A virus, modified virus, or viral vector. In some embodiments, the target includes an influenza B virus, modified virus, or viral vector. In some embodiments, the target includes an influenza C virus, modified virus, or viral vector. In some embodiments, the target includes an influenza D virus, modified virus, or viral vector.

A viral vector is a unidirectional non-propagating gene delivery system. The gene of a viral vector is encapsulated in shell. The shell may be a capsid or an envelope (e.g., a lipid bilayer). Viral vectors may include proteins displayed on the surface of the shell such that the proteins are able to interact with proteins and/or other molecules exterior to the viral vector. For example, a viral vector may include hemagglutinin displayed on the surface of the shell. The proteins displayed on the surface of a viral vector may be bound to shell and/or partially lodged within the shell.

In some embodiments, the target comprises hemagglutinin and the hemagglutinin is eHA1 (from H1N1 A/California/07/2009 virus), H3 type A or recombinant H3 Type A, HA from the B/Victoria virus or recombinant HA from the B/Victoria virus, or HA from the B/Yamagata-like virus or recombinant HA from the B/Yamagata-like virus. In some embodiments, the target comprises hemagglutinin and the hemagglutinin is H1, H2, H3, H4, H5, H6, H7, H8, H9, H10, H11, H12, H13, H14, H15, H16, H17, H18, or a fragment thereof from the influenza A virus. In some embodiments, the target includes HA1 subunit (eHA1) of the influenza A virus subtype H1N1.

The separation media of the present disclosure include a plurality of separation ligands immobilized on a support substrate. The separation ligands include one or more separation groups. A separation group is a chemical group that facilitates the isolation of a target molecule from an isolation solution. Facilitation of separation may be in the form of a chemical group to which the target molecule binds; a chemical group that allows for increased density of the affinity group-target molecule interaction and/or increases the target molecule attraction to the support substrate; or a chemical group that blocks a reactive group from covalently modifying the target molecule during contact with the separation media; or a combination thereof. A separation group may be an affinity group, an assistance group, or a capping group. The separation media includes a plurality of separation ligands that include an affinity group. In addition to the plurality of separation ligands that include an affinity group, the separation media may include a plurality of separation ligands that include an assistance group; a plurality of separation ligands that include a capping group; or both.

A support substrate is the base material for the separation media. The support substrate provides a platform for which the separation ligands are immobilized. The support substrate includes at least one membrane. In some embodiments, the support substrate is the at least one membrane. In some embodiments, the support substrate includes two or more membranes arranged in a stacked configuration. In addition to the at least one membrane, the support substrate may include additional layers such as hydrogels, woven fibrous materials (i.e., a material made by the interlacing of multiple fibers), nonwoven fibrous materials (i.e., a material made from one or more fibers that are bound together through chemical, physical, heat, or mechanical treatment); or combinations thereof. Such additional layers may impart rigidity and structure to the support substrate. In some embodiments, the support substrate includes a functionalized material that is deposited on the surface of the at least one membrane. The functionalized material may provide reactive handles to which the separation ligands may be reacted to be immobilized to the support substrate. In embodiments where the separation media includes multiple layers, the layers may be laminated.

Any layer of the support substrate may be made of any suitable material. A suitable support substrate material is a material that is porous so as to allow the isolation solution to pass through the support substrate. In some embodiments, a suitable support substrate material is a material that does not chemically alter the target molecule; that is, does not react with the target molecule to add, remove, or transform chemical groups on the target molecule. Additionally, in some embodiments, a suitable support substrate is a material that does not react with the target molecule, or other molecules in the isolation solution, to form a covalent bond which would permanently immobilize said molecule to the support substrate.

The support substrate includes at least one membrane. A membrane is understood as a sheet of material with a continuous pathway of polymeric material in all dimensions. The membrane may be made of any suitable support substrate material. Examples of suitable support substrate membrane materials include polyolefins; polyethersulfone; poly(tetrafluoroethylene); nylon; fiberglass; hydrogels; polyvinyl alcohol; natural polymers such as cellulose, cellulose ester, cellulose acetate, regenerated cellulose, cellulosic nanofiber, cellulose derivatives, agarose, chitosan; polyethylene; polyester; polysulfone; expanded polytetrafluoroethylene (ePTFE), polyvinylidene fluoride; polyamide (Nylon); polyacrylonitrile; polycarbonate; and any combination thereof.

In some embodiments, the support substrate or membrane itself is functionalized prior to immobilizing the separation ligands. Functionalization of the membrane may be done to install reactive handles (e.g., a support substrate reactive handle as discussed elsewhere herein) on the membrane. The reactive handles react with cooperative reactive handles on the separation ligands to form a covalent bond thereby immobilizing the separation ligands on the support substrate (as discussed elsewhere herein). Functionalization may be accomplished by plasma treatment, corona treatment, and the like.

In some embodiments, the support substrate includes a functionalized layer. In some embodiments, the functionalized layer is a membrane. A functionalized layer is a material disposed on the surface of a support substrate layer (e.g., disposed on the surface of the at least one membrane) and includes the support substrate reactive handles that may be used for separation ligand immobilization. A functionalized layer may be covalently attached to the support substrate; adhered to the support substrate through electrostatic forces, hydrogen-bonding, and/or Van der Waals forces; laminated to the support substrate; or simply contacting the support substrate. A functionalized layer may be deposited on the surface a support substrate (e.g., on the surface of the at least one membrane) using a variety of deposition techniques such as chemical vapor deposition, dip coating, spray coating, electrospinning, and the like.

In some embodiments, the functionalized layer is a polymer that is disposed onto the support surface using a grafting on or grafting from polymerization technique. The terms “grafting on,” “grafting onto,” and “grafted onto” refer to already formed polymer chains that adsorb or covalently attach to a surface (e.g., a support substrate surface). The terms “grafting from” or “grafted from” refer to a polymer chain that is imitated and grown from a surface (e.g., a support substrate surface). Any suitable polymer may be grafted on or grafted from a support substrate to from a functionalized layer. Suitable polymers are those that include a functional group that includes a reactive handle that allows for attachment of separation ligands to the support substrate. The reactive handle is not the polymerizable group, but instead is a group that remains intact following polymerization. Example polymers that include a reactive handle or a functional group that can be converted to a reactive handle (i.e., support substrate reactive handle) include carboxylic acids, amines, alcohols, epoxides, amides, azide, alkynes, and the like. Examples of monomers that can be used to form such polymers include vinyl alcohol, hydroxy functional acrylates (e.g., 2-hydroxyehtyl acrylate and 4-hydroxybutyl acrylate), hydroxy functional methacrylate (e.g., hydroxyethyl methacrylate), epoxy containing monomers, and hydroxy functional acrylamides (e.g., N-hydroxyethyl acrylamide). Examples of specific polymers that may be grafted on or grafted from a support substrate include polydopamine, poly(vinyl alcohol), poly(acrylic acid), poly(glycidyl methacrylate, and poly 2-hydroxyethyl acrylate (formed from 2-hydroxyethyl acrylate monomers). Graft on and graft from polymerization may be accomplished using suitable technique such addition polymerization (e.g., free radical polymerization such as atom transfer radical polymerization (ATRP) and reversible addition fragmentation chain transfer (RAFT) polymerization; anionic polymerization; and cationic polymerization) or condensation polymerization. In embodiments, where the polymer is grafted from the support substrate, an initiator is first coupled to the support substrate (e.g., through an OH group on the support substrate). Any suitable initiator may be used, for example, 2-bromo-2-methylpropionyl bromide (BiBB).

The membranes of the support substrate are porous and can have an average pore size, as measure by a capillary flow porometer, of 10 micrometer (μm) or less, 5 or less, 2 μm or less, 1 μm or less, 0.6 μm or less, 0.5 μm or less, 0.45 μm or less, or 0.2 μm or less. The membrane may have an average pore size of 0.1 μm or greater, 0.2 μm or greater, 0.45 μm or greater, 0.5 μm or greater, 0.6 μm or greater, 0.7 μm or greater, or 1 μm or greater. The membrane may have an average pore size ranging from about 0.1 μm to 10.0 μm, 0.1 μm to 0.2 μm, 0.1 μm to 0.45 μm, 0.1 μm to 0.5 μm, 0.1 μm to 1 μm, 0.2 μm to 0.45, 0.2 μm to 0.50, 0.2 μm to 1 μm, 0.2 μm to 2 μm, 0.2 μm to 10 μm, 0.45 μm to 1 μm, 0.45 μm to 2 μm, 0.45 μm to 10 μm, 1 μm to 2 μm, or 1 μm to 5 μm. In some embodiments, the support substrate has an average pore size of 0.1 μm to 0.5 μm, 0.1 μm to 0.6 μm, 0.1 μm to 0.3 μm, or 0.4 μm to 0.6 μm.

In some embodiments, the support membrane includes cellulose such as regenerated cellulose, cellulose acetate, or cellulose ester. In some such embodiments, the support membrane has an average pore size 0.1 μm to 0.5 μm, 0.1 μm to 0.6 μm, 0.1 μm to 0.3 μm, or 0.4 μm to 0.6 μm.

The membrane may have a thickness of 500 μm or greater, 250 μm or greater, 100 μm or greater, 80 μm or greater, 50 μm or greater, or 30 μm or greater. The membrane may have a thickness of 2500 μm or less, 1000 μm or less, 500 μm or less, 250 μm or less, or 100 μm or less. The thickness of the membrane may be in a range of 30 μm to 500 μm, 50 μm to 500 μm, 80 μm to 500 μm, 100 μm to 500 μm, 250 μm to 500 μm, 30 μm to 250 μm, 50 μm to 250 μm, 80 μm to 250 μm, 100 μm to 2500 μm, 30 μm to 100 μm, 50 μm to 100 μm, or 80 μm to 100 μm.

In some embodiments, the support substrate includes multiple membranes stacked in a multilayer arrangement to increase capacity or selectivity of the separation media for a given application. The multilayer membrane configuration (i.e., only considering the membrane layers of a support substrate) maybe have a thickens of 10,000 micrometers (um) or less, 7,500 μm or less, 5,000 μm or less, 4,000 μm or less, 3,000 μm or less, 2,500 μm or less, 2,000 μm or less, 1,000 μm or less, 750 μm or less, 500 μm or less, 400 μm or less, or 300 μm or less. The stacked arrangement of membranes may have a thickness ranging from 70 μm to 10,000 μm, 70 μm to 100 μm, 70 μm to 200 μm, 70 μm to 300 μm, 70 μm to 400 μm, 70 μm to 500 μm, 70 μm to 750 μm, 70 μm to 1,000 μm, 70 μm to 2,000 μm, 70 μm to 3,000 μm, 70 μm to 4,000 μm, 70 μm to 5,000 μm, 250 μm to 300 μm, 250 μm to 400 μm, 250 μm to 500 μm, 250 μm to 750 μm, 250 μm to 1,000 μm, 250 to 2,000 μm, 250 to 3,000 μm, 250 to 4,000 μm, 250 to 5,000 μm, 500 μm to 1,000 μm, 500 μm to 2,000 μm, 500 μm to 3,000 μm, 500 μm to 4,000 μm, or 500 μm to 5,000 μm in thickness.

In some embodiments, the membrane is a regenerated cellulose membrane having a pore size of between 0.2 μm and 5.0 μm, a thickness of between 70 μm and 2,000 μm. Such membranes may be in a stacked arrangement approximately 70 μm to 10,000 μm in thickness.

The support substrate may include or be a microfiltration membrane. Microfiltration membranes are typically created through a phase inversion process or an expansion process. Typical materials used to prepare membranes include polyethersulfone (PES), nylon, polyvinylidene fluoride (PVDF), cellulose acetate, regenerated cellulose, polypropylene, and expanded polytetrafluoroethylene (ePTFE).

The separation media includes a plurality of separation ligands that include an affinity group. In addition to the plurality of separation ligands that include an affinity group, the separation media may include a plurality of separation ligands that include an assistance group; a plurality of ligands that include a capping group; or both.

An affinity group is a chemical group that is bound by the target molecule. Stated differently, an affinity group is a chemical group that binds the target molecule. The affinity group may include a carbohydrate or be a small molecule. In some embodiments, the affinity group includes a carbohydrate. In other embodiments, the affinity group includes carbohydrate containing protein. The targets include a carbohydrate binding domain that can bind to the small molecule or carbohydrate of the affinity group thereby temporarily immobilizing the target molecule to the separation media. The carbohydrate may be a monosaccharide, a disaccharide, or a glycan.

A monosaccharide is the simplest carbohydrate form. Monosaccharide can have various chemical compositions and configurations. Generally, a monosaccharide includes carbon, hydrogen, and oxygen atoms often according to the empirical formula Cₘ(H₂O)ₙ where m and n are integers that may or may not be the same (i.e., the hydrogen-oxygen ratio is 2:1). However, not all monosaccharides follow the empirical formula such as, for example, uronic acid and deoxy-sugars (e.g., fucose, deoxyribose, fuculose, and rhamnose), and dideoxy sugars (e.g., colitose and abequose). Additionally, monosaccharides may include one or more substituents or modifications that make is such that the monosaccharide no longer follows the empirical formula. Example substituent groups and modifications include acetyl (Ac); D-alanyl (Ala), N-acetyl-D-alanyl (Ala2Ac); N-acetimidoyl (Am); N-(N-methyl-acetimidoyl) (AmMe); N-(N,N-dimethyl-acetimidoyl) (AmMez); formyl (Fo); glycolyl (Gc); N-acetyl-glutaminyl (Gln2Ac); N-methyl-5-glutamyl (5Glu2Me); glycyl (Gly); glyceryl (Gr); 2,3-di-O-methyl-glyceryl (Gr2,3Mez); 4-hydroxybutyryl (4Hb); 3,4-dihydroxybutyryl (3,4Hb); (R)-3-hydroxybutyryl (3RHb); (S)-3-hydroxybutyryl (3sHb); lactyl (Lt); methyl (Me); amino (N); N-acetyl (NAc); phosphate (P); pyruvyl (Py); 1-carboxyethylidene (Pyr); sulfate (S); and tauryl (Tau). Table 1 shows examples of monosaccharides.

Each monosaccharide may have a variety of stereoisomers. For example, monosaccharides are often classified as D or L depending on the stereochemistry of the stereocenter that is the farthest away from the anomeric carbon. Additionally, monosaccharides can exist as anomers (α-anomer and β-anomer). An anomer is a pair of stereoisomers that are identical accept at the configuration of the anomeric carbon. A single monosaccharide may exist as an a-anomer or a β-anomer.

TABLE 1
Example monosaccharides

| Monosaccharide name | Monosaccharide name | Monosaccharide name |
|---|---|---|
| 4-epi-Legionaminic | L-Fucose | N-Acetyl-D- |
| acid | | mannosamine |
| 6-Deoxy-L-altrose | N-Acetyl-L-fucosamine | Muramic acid |
| N-Acetyl-6-deoxy-L- | D-Galactose | N-Acetylmuramic |
| altrosamine | | acid |
| 6-Deoxy-D-gulose | D-Galacturonic acid | N-Glycolylmuramic |
| | | acid |
| 6-Deoxy-D-talose | D-Galactosamine | Neuraminic acid |
| N-Acetyl-6-deoxy-D- | N-Acetyl-D- | N-Acetylneuraminic |
| talosamine | galactosamine | acid |
| 8-epi-Acinetaminic | D-Glucose | N-Glycolylneuraminic |
| acid | | acid |
| 8-epi-Legionaminic | D-Glucuronic acid | Olivose |
| acid | | |
| Abequose | D-Glucosamine | Paratose |
| Acinetaminic acid | N-Acetyl-D-glucosamine | Pseudaminic acid |
| D-Allose | D-Gulose | D-Psicose |
| D-Alluronic acid | D-Guluronic acid | D-Quinovose |
| D-Allosamine | D-Gulosamine | N-Acetyl- |
| | | D-quinovosamine |
| N-Acetyl-D-allosamine | N-Acetyl-D-gulosamine | L-Rhamnose |
| L-Altrose | L-Idose | N-Acetyl-L- |
| | | rhamnosamine |
| L-Altruronic acid | L-Iduronic acid | D-Ribose |
| L-Altrosamine | L-Idosamine | Sialic acid |
| N-Acetyl-L-altrosamine | N-Acetyl-L-idosamine | L-Sorbose |
| L-Apiose | 2-Keto-3-deoxy-nononic | D-Tagatose |
| | acid | |
| L-Arabinose | 3-Deoxy-D-manno- | D-Talose |
| | octulosonic acid | |
| Bacillosamine | Legionaminic acid | D-Taluronic acid |
| Colitose | L-glycero-D-manno- | D-Talosamine |
| | Heptose | |
| D-glycero-D-manno- | D-Lyxose | N-Acetyl-D- |
| Heptose | | talosamine |
| 3-Deoxy-D-lyxo- | D-Mannose | Tyvelose |
| heptulosaric acid | | |
| D-Digitoxose | D-Mannuronic acid | D-Xylose |
| D-Fructose | D-Mannosamine | |

Monosaccharides may be covalently linked to form larger molecules such as disaccharides or glycans. Monosaccharides may be linked through glycosidic bonds; that is, covalent bonds between adjacent monosaccharides of a disaccharide or glycan. Glycosidic bonds may also be used to describe a monosaccharide or a glycan that is covalently linked to a different molecule such as a protein. In disaccharides and glycans, a glycosidic linkage couples the anomeric carbon of a first saccharide to a carbon of an adjacent saccharide through and O-type (ether linkage), N-type (amine linkage), or S-type (thioether linkage) linkage. Glycosidic linkages may take on several configurations depending on how two monosaccharides are connected. The configuration of the linkage is denoted by listing the anomeric carbon number from which the bond originates first followed by the carbon number of the second monosaccharide to which the anomeric carbon of the first monosaccharide is connected. For example, a first monosaccharide and a second monosaccharide may have a C1 to C4 glycosidic linkage; that is, the anomeric carbon (C1) of the first monosaccharide is coupled to the carbon in position 4 (C4) of the second monosaccharide. Additionally, the stereochemistry configuration of the glycosidic linkage may vary depending on the configuration of the anomeric carbon that covalently links the first monosaccharide to the second monosaccharide. If the first monosaccharide is an α-anomer, the glycosidic linkage is and a linkage. If the first monosaccharide is a β-anomer (beta-anomer), the glycosidic linkage is beta (B) linkage. The monosaccharide compostion and the configuration of the linkage of the monosaccharaides leads to polysaccharides with higher order structure. Disaccharides and polysaccharides may be homopolysaccharides (or homoglycans) or may be heterosaccharides (or heteroglycans). Homopolysaccharides include a single type of monosaccharide. In contrast, heteropolysaccharides include two or more different types of monosaccharides. Glycans may be in a linear or branched configuration. A linear glycan has a straight chain of linked monosaccharides. Examples of linear glycans include cellulose and chitin. A branched glycan is a glycan that has a glycan backbone and one or more branches off the glycan backbone.

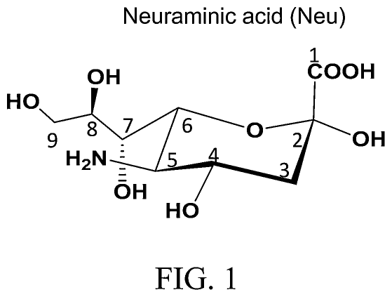

Sialic acids are a class of saccharides (a subclass of nonulsonic acids) that help regulate cell-cell interaction, cell signaling, carbohydrate-cell interactions, cell aggregation, immune reactions, reproduction, and developmental processes. Additionally, sialic acids have been found to enable infectious disease infection (e.g., viruses and bacteria) and tumor growth. Sialic acids (also called neuraminic acids) are alpha-keto acid (possess a ketone and carboxylic acid group) saccharides that have a nine carbon backbone. FIG. 1 shows the structure of the neuraminic acid (Neu), the simplest sialic acid from which other sialic acids are derived. FIG. 1 also shows the carbon numbering system of sialic acids. There are numerous sialic acids that differ in structures. Examples of sialic acid monosaccharides are shown in Table 2. Additionally, there are “sialic acid-like” monosaccharides which are de dideoxy-nonulosonic acids (deoxy at C3 and C 9). Examples of sialic acid like monosaccharides are given in Table 3.

TABLE 2
Example sialic acid monosaccharides

| Sialic Acid Name | Abbreviation |
|---|---|
| 5-Amino-3,5-dideoxy-D-glycero- | Neu |
| D-galacto-non-2-ulosonic acid/ | |
| neuraminic acid | |
| Neuraminic acid 1,5-lactam | Neu1,5lactam |
| 5-N-Acetyl-neuraminic acid | Neu5Ac |
| (N-Acetylneuraminic acid) | |
| 5-N-Acetyl-4-O-acetyl-neuraminic acid | Neu4,5Ac2 |
| 5-N-Acetyl-7-O-acetyl-neuraminic acid | Neu5,7Ac2 |
| 5-N-Acetyl-8-O-acetyl-neuraminic acid | Neu5,8Ac2 |
| 5-N-Acetyl-9-O-acetyl-neuraminic acid | Neu5,9Ac2 |
| 5-N-Acetyl-4,9-di-O-acetyl-neuraminic acid | Neu4,5,9Ac3 |
| 5-N-Acetyl-7,8-di-O-acetyl-neuraminic acid | Neu5,7,8Ac3 |
| 5-N-Acetyl-7,9-di-O-acetyl-neuraminic acid | Neu5,7,9Ac3 |
| 5-N-Acetyl-8,9-di-O-acetyl-neuraminic acid | Neu5,8,9Ac3 |
| 5-N-Acetyl-4,7,9-tri-O-acetyl-neuraminic acid | Neu4,5,7,9Ac4 |
| 5-N-Acetyl-7,8,9-tri-O-acetyl-neuraminic acid | Neu5,7,8,9Ac4 |
| 5-N-Acetyl-4,7,8,9-tetra-O-acetyl- | Neu4,5,7,8,9Ac5 |
| neuraminic acid | |
| 5-N-Acetyl-4-O-glycolyl-neuraminic acid | Neu5 Ac4Gc |
| 5-N-Acetyl-7-O-glycolyl-neuraminic acid | Neu5Ac7Gc |
| 5-N-Acetyl-9-O-lactyl-neuraminic acid | Neu5Ac9Lt |
| 5-N-Acetyl-4-O-acetyl-9-O-lactyl- | Neu4,5Ac29Lt |
| neuraminic acid | |
| 5-N-Acetyl-7-O-acetyl-9-O-lactyl- | Neu5,7Ac29Lt |
| neuraminic acid | |
| 5-N-Acetyl-8-O-acetyl-9- | Neu5,8Ac29Lt |
| O-lactyl-neuraminic acid | |
| 5-N-Acetyl-8-O-methyl-neuraminic acid | Neu5Ac8Me |
| 5-N-Acetyl-4-O-acetyl-8- | Neu4,5Ac28Me |
| O-methyl-neuraminic acid | |
| 5-N-Acetyl-9-O-acetyl-8- | Neu5,9Ac28Me |
| O-methyl-neuraminic acid | |
| 5-N-Acetyl-9-O-methyl-neuraminic acid | Neu5Ac9Me |
| 5-N-Acetyl-4-O-sulpho-neuraminic acid | Neu5 Ac4S |
| 5-N-Acetyl-8-O-sulpho-neuraminic acid | Neu5Ac8S |
| 5-N-Acetyl-4-O-acetyl-8- | Neu4,5Ac28S |
| O-sulpho-neuraminic acid | |
| 5-N-Acetyl-9-O-phospho-neuraminic acid | Neu5Ac9P |
| 5-N-Acetyl-2-deoxy-2,3- | Neu2en5Ac |
| didehydro-neuraminic acid | |
| 5-N-Acetyl-9-O-acetyl-2-deoxy- | Neu2en5,9Ac2 |
| 2,3-didehydro-neuraminic acid | |
| 5-N-Acetyl-2-deoxy-2,3-didehydro- | Neu2en5Ac9Lt |
| 9-O-lactyl-neuraminic acid | |
| 5-N-Acetyl-2,7-anhydro-neuraminic acid | Neu2,7an5 Ac |
| 5-N-Acetyl-4,8-anhydro-neuraminic acid | Neu4,8an5Ac |
| 5-N-Acetyl-neuraminic acid 1,7-lactone | Neu1,7lactone5 Ac |
| 5-N-Acetyl-9-O-acetyl-neuraminic | Neu1,7lactone5,9Ac2 |
| acid 1,7-lactone | |
| 5-N-Acetyl-4,9-di-O-acetyl-neuraminic | Neu1,7lactone4,5,9Ac3 |
| acid 1,7-lactone | |
| 1-Tauryl 5-N-acetyl-neuraminic amide | Neu5 Ac1Tau |
| 5-N-Glycolyl-neuraminic acid | Neu5Gc |
| (N-Glycolylneuraminic acid) | |
| 4-O-Acetyl-5-N-glycolyl-neuraminic acid | Neu4Ac5Gc |
| 7-O-Acetyl-5-N-glycolyl-neuraminic acid | Neu7Ac5Gc |
| 8-O-Acetyl-5-N-glycolyl-neuraminic acid | Neu8Ac5Gc |
| 9-O-Acetyl-5-N-glycolyl-neuraminic acid | Neu9Ac5Gc |
| 4,7-Di-O-acetyl-5-N-glycolyl-neuraminic acid | Neu4,7Ac25Gc |
| 4,9-Di-O-acetyl-5-N-glycolyl-neuraminic acid | Neu4,9Ac25Gc |
| 7,9-Di-O-acetyl-5-N-glycolyl-neuraminic acid | Neu7,9Ac25Gc |
| 8,9-Di-O-acetyl-5-N-glycolyl-neuraminic acid | Neu8,9Ac25Gc |
| 4,7,9-Tri-O-acetyl-5-N-glycolyl-neuraminic acid | Neu4,7,9Ac35Gc |
| 7,8,9-Tri-O-acetyl-5-N-glycolyl-neuraminic acid | Neu7,8,9Ac35Gc |
| 4,7,8,9-Tetra-O-acetyl-5-N- | Neu4,7,8,9Ac45Gc |
| glycolyl-neuraminic acid | |
| 5-N-Glycolyl-9-O-lactyl-neuraminic acid | Neu5Gc9Lt |
| 4-O-Acetyl-5-N-glycolyl-9- | Neu4Ac5Gc9Lt |
| O-lactyl-neuraminic acid | |
| 7-O-Acetyl-5-N-glycolyl-9- | Neu7Ac5Gc9Lt |
| O-lactyl-neuraminic acid | |
| 8-O-Acetyl-5-N-glycolyl-9- | Neu8Ac5Gc9Lt |
| O-lactyl-neuraminic acid | |
| 4,7-Di-O-acetyl-5-N-glycolyl- | Neu4,7Ac25Gc9Lt |
| 9-O-lactyl-neuraminic acid | |
| 7,8-Di-O-acetyl-5-N-glycolyl- | Neu7,8Ac25Gc9Lt |
| 9-O-lactyl-neuraminic acid | |
| 5-N-Glycolyl-8-O-methyl-neuraminic acid | Neu5Gc8Me |
| 4-O-Acetyl-5-N-glycolyl-8- | Neu4Ac5Gc8Me |
| O-methyl-neuraminic acid | |
| 7-O-Acetyl-5-N-glycolyl-8- | Neu7Ac5Gc8Me |
| O-methyl-neuraminic acid | |
| 9-O-Acetyl-5-N-glycolyl-8- | Neu9Ac5Gc8Me |
| O-methyl-neuraminic acid | |
| 4,7-Di-O-acetyl-5-N-glycolyl- | Neu4,7Ac25Gc8Me |
| 8-O-methyl-neuraminic acid | |
| 7,9-Di-O-acetyl-5-N-glycolyl- | Neu7,9Ac25Gc8Me |
| 8-O-methyl-neuraminic acid | |
| 5-N-Glycolyl-9-O-methyl-neuraminic acid | Neu5Gc9Me |
| 5-N-Glycolyl-8-O-sulpho-neuraminic acid | Neu5Gc8S |
| 5-N-Glycolyl-9-O-sulpho-neuraminic acid | Neu5Gc9S |
| 5-N-(O-Acetyl)glycolyl-neuraminic acid | Neu5(Gc2Ac) |
| 5-N-(O-Methyl)glycolyl-neuraminic acid | Neu5(Gc2Me) |
| 2-Deoxy-2,3-didehydro-5- | Neu2en5Gc |
| N-glycolyl-neuraminic acid | |
| 9-O-Acetyl-2-deoxy-2,3-didehydro- | Neu2en9Ac5Gc |
| 5-N-glycolyl-neuraminic acid | |
| 2-Deoxy-2,3-didehydro-5-N-glycolyl- | Neu2en5Gc9Lt |
| 9-O-lactyl-neuraminic acid | |
| 2-Deoxy-2,3-didehydro-5-N-glycolyl- | Neu2en5Gc8Me |
| 8-O-methyl-neuraminic acid | |
| 2,7-Anhydro-5-N-glycolyl-neuraminic acid | Neu2,7an5Gc |
| 2,7-Anhydro-5-N-glycolyl- | Neu2,7an5Gc8Me |
| 8-O-methyl-neuraminic acid | |
| 4,8-Anhydro-5-N-glycolyl-neuraminic acid | Neu4,8an5Gc |
| 5-N-Glycolyl-neuraminic acid 1,7-lactone | Neu1,7lactone5Gc |
| 9-O-Acetyl-5-N-glycolyl-neuraminic | Neu1,7lactone9Ac5Gc |
| acid 1,7-lactone | |
| 7-Acetamido-9-O-acetyl-7-deoxy- | Neu9Ac5Gc7NAc |
| 5-N-glycolyl-neuraminic acid | |
| 7-Acetamido-8,9-di-O-acetyl-7- | Neu8,9Ac25Gc7NAc |
| deoxy-5-N-glycolyl-neuraminic acid | |
| 3-Deoxy-D-glycero-D-galacto- | Kdn |
| non-2-ulosonic acid/2-keto-3- | |
| deoxy-nononic acid | |
| 5-O-Acetyl-2-keto-3-deoxy-nononic acid | Kdn5Ac |
| 7-O-Acetyl-2-keto-3-deoxy-nononic acid | Kdn7Ac |
| 8-O-Acetyl-2-keto-3-deoxy-nononic acid | Kdn8Ac |
| 9-O-Acetyl-2-keto-3-deoxy-nononic acid | Kdn9Ac |
| 4,5-Di-O-acetyl-2-keto-3-deoxy-nononic acid | Kdn4,5Ac2 |
| 4,7-Di-O-acetyl-2-keto-3-deoxy-nononic acid | Kdn4,7Ac2 |
| 5,9-Di-O-acetyl-2-keto-3-deoxy-nononic acid | Kdn5,9Ac2 |
| 7,9-Di-O-acetyl-2-keto-3-deoxy-nononic acid | Kdn7,9Ac2 |
| 8,9-Di-O-acetyl-2-keto-3-deoxy-nononic acid | Kdn8,9Ac2 |
| 2-Keto-3-deoxy-4-O-methyl-nononic acid | Kdn4Me |
| 2-Keto-3-deoxy-5-O-methyl-nononic acid | Kdn5Me |
| 2-Keto-3-deoxy-9-O-methyl-nononic acid | Kdn9Me |
| (R)-7,9-O-[1-Carboxyethylidene]- | Kdn7,9PyrR |
| 2-keto-3-deoxy-nononic acid | |
| 2-Keto-3-deoxy-9-O-phospho-nononic acid | Kdn9 |

TABLE 3
Example sialic acid-like monosaccharides

| Sialic Acid-Like Monosaccharide Name | Abbreviation |
|---|---|
| 5,7-Diamino-3,5,7,9-tetradeoxy- | Pse |
| L-glycero-L-manno-non-2-ulosonic | |
| acid/pseudaminic acid | |
| 5,7-Di-N-acetyl-pseudaminic acid | Pse5,7Ac2 |
| 5,7-Di-N-acetyl-4-O-acetyl-pseudaminic acid | Pse4,5,7Ac3 |
| 5,7-Di-N-acetyl-8-O-acetyl-pseudaminic acid | Pse5,7,8Ac3 |
| 5,7-Di-N-acetyl-8-O-glycyl-pseudaminic acid | Pse5,7Ac28Gly |
| 5,7-Di-N-glyceryl-pseudaminic acid | Pse5,7Gr2 |
| 5-N-Acetimidoyl-7-N-acetyl-pseudaminic acid | Pse7Ac5Am |
| 5-N-Acetimidoyl-7-N-acetyl-8- | Pse7,8Ac25Am |
| O-acetyl-pseudaminic acid | |
| 5-N-Acetimidoyl-7-N-acetyl- | Pse7Ac5Am8(Gln2Ac) |
| 8-O-(N-acetyl-glutaminyl)- | |
| pseudaminic acid | |
| 5-N-Acetyl-7-N-formyl-pseudaminic acid | Pse5Ac7Fo |
| 5-N-Acetyl-7-N-L-glyceryl-pseudaminic acid | Pse5Ac7Gr |
| 5-N-Acetyl-7-N-[(R)-3- | Pse5Ac7(3RHb) |
| hydroxybutyryl]-pseudaminic acid | |
| 5-N-Acetyl-7-N-[(R)-3-hydroxybutyryl]- | Pse4,5Ac27(3RHb) |
| 4-O-acetyl-pseudaminic acid | |
| 5-N-Acetyl-7-N-[(S)-3- | Pse5Ac7(3SHb) |
| hydroxybutyryl]-pseudaminic acid | |
| 5-N-Acetyl-7-N-(4- | Pse5Ac7(4Hb) |
| hydroxybutyryl)-pseudaminic acid | |
| 5-N-Acetyl-7-N-(3,4- | Pse5Ac7(3,4Hb) |
| dihydroxybutyryl)-pseudaminic acid | |
| 7-N-Acetimidoyl-5-N-acetyl-pseudaminic acid | Pse5Ac7Am |
| 5-N-Acetimidoyl-7-N-glyceryl- | Pse5Am7Gr |
| pseudaminic acida | |
| 7-N-Acetimidoyl-5-N-(2,3-di-O- | Pse7Am5(Gr2,3Me2) |
| methyl-glyceryl)-pseudaminic acid | |
| 7-N-Acetyl-5-N-(3- | Pse7Ac5(3Hb) |
| hydroxybutyryl)-pseudaminic acid | |
| 7-N-Acetyl-5-N-(2,3-di-O-methyl- | Pse7Ac5(Gr2,3Me2) |
| glyceryl)-pseudaminic acid | |
| 7-N-Formyl-5-N-[(R)-3- | Pse7Fo5(3RHb) |
| hydroxybutyryl]-pseudaminic acid | |
| 5,7-Diamino-3,5,7,9-tetradeoxy- | Leg |
| D-glycero-D-galacto-non-2-ulosonic | |
| acid/legionaminic acid | |
| 5,7-Di-N-acetyl-legionaminic acid | Leg5,7Ac2 |
| 5,7-Di-N-acetyl-4-O-acetyl-legionaminic acid | Leg4,5,7Ac3 |
| 5,7-Di-N-acetyl-8-amino-8- | Leg5,7Ac28N |
| deoxy-legionaminic acid | |
| 5-N-Acetimidoyl-7-N-acetyl-legionaminic acid | Leg7Ac5Am |
| 5-N-Acetimidoyl-7-N-acetyl-8- | Leg7,8Ac25Am |
| O-acetyl-legionaminic acid | |
| 5-N-Acetimidoyl-7-N-acetyl-5- | Leg7Ac5Am5Me |
| N-methyl-legionaminic acid | |
| 5-N-(N-Methyl-acetimidoyl)-7- | Leg7Ac5AmMe |
| N-acetyl-legionaminic acid | |
| 5-N-(N,N-Dimethyl-acetimidoyl)- | Leg7Ac5AmMe2 |
| 7-N-acetyl-legionaminic acid | |
| 5-N-Acetimidoyl-7-N-acetyl-8-O- | Leg7,8Ac25Am5Me |
| acetyl-5-N-methyl-legionaminic | |
| acid | |
| 5-N-(N,N-Dimethyl-acetimidoyl)- | Leg7,8Ac25AmMe2 |
| 7-N-acetyl-8-O-acetyl- | |
| legionaminic acid | |
| 5-N-Acetyl-7-N-(N-acetyl- | Leg5Ac7(Ala2Ac) |
| D-alanyl)-legionaminic acid | |
| 5-N-Acetyl-7-N-(D-alanyl)-legionaminic acid | Leg5Ac7Ala |
| 7-N-Acetyl-5-N-formyl-legionaminic acid | Leg7Ac5Fo |
| 7-N-Acetyl-5-N-[(S)-3- | Leg7Ac5(3SHb) |
| hydroxybutyryl]-legionaminic acidd | |
| 7-N-Acetyl-5-N-(N-methyl-5- | Leg7Ac5(5Glu2Me) |
| glutamyl)-legionaminic acid | |
| 5,7-Diamino-3,5,7,9-tetradeoxy- | 4Leg |
| D-glycero-D-talo-non-2-ulosonic | |
| acid/4-epi-legionaminic acid | |
| 5,7-Di-N-acetyl-4-epi-legionaminic acid | 4eLeg5,7Ac2 |
| 5,7-Di-N-acetyl-8-O-acetyl- | 4eLeg5,7,8Ac3 |
| 4-epi-legionaminic acid | |
| 5-N-Acetimidoyl-7-N-acetyl- | 4eLeg7Ac5Am |
| 4-epi-legionaminic acid | |
| 5-N-Acetimidoyl-7-N-acetyl-8- | 4eLeg7,8Ac25Am |
| O-acetyl-4-epi-legionaminic acid | |
| 5,7-Diamino-3,5,7,9-tetradeoxy- | 8eLeg |
| L-glycero-D-galacto-non-2-ulosonic | |
| acid/8-epi-legionaminic acid | |
| 5,7-Di-N-acetyl-8-epi-legionaminic acid | 8eLeg5,7Ac2 |
| 5,7-Di-N-acetyl-8-O-acetyl- | 8eLeg5,7,8Ac3 |
| 8-epi-legionaminic acid | |
| 5-N-Acetimidoyl-7-N-acetyl- | 8eLeg7Ac5Am |
| 8-epi-legionaminic acid | |
| 7-N-Acetimidoyl-5-N-acetyl- | 8eLeg5Ac7Am |
| 8-epi-legionaminic acid | |
| 7-N-Acetimidoyl-5-N-acetyl-8- | 8eLeg5,8Ac27Am |
| O-acetyl-8-epi-legionaminic acid | |
| 7-N-Acetyl-5-N-[(R)-3-hydroxybutyryl]- | 8eLeg7Ac5(3RHb) |
| 8-epi-legionaminic acid | |
| 7-N-Acetyl-5-N-(4-hydroxybutyryl)- | 8eLeg7Ac5(4Hb) |
| 8-epi-legionaminic acid | |
| 5,7-Diamino-3,5,7,9-tetradeoxy-L- | Aci |
| glycero-L-altro-non-2-ulosonic | |
| acid/acinetaminic acid | |
| 5,7-Di-N-acetyl-acinetaminic acid | Aci5,7Ac2 |
| 5,7-Diamino-3,5,7,9-tetradeoxy- | 8eAci |
| D-glycero-L-altro-non-2-ulosonic | |
| acid/8-epi-acinetaminic acid | |
| 5,7-Di-N-acetyl-8-epi-acinetaminic acid | 8eAci5,7Ac2 |
| Some related 9-deoxy-non-2-ulosonic acids | |
| 5- or 7-Acetamido-,7- or 5- | |
| (3-hydroxybutyramido)-5,7,9-trideoxy- | |
| non-2-ulosonic acid | |
| 5-Acetamido-7-[(S)-3- | |
| hydroxybutyramido]-8-amino-3,5,7,8,9- | |
| pentadeoxy-L-glycero-L-manno- | |
| or D-glycero-L-manno-non-2- | |
| ulosonic acid | |
| 5-Acetamidino-3,5,9-trideoxy- | |
| L-glycero-L-gluco-non-2-ulosonic | |
| acid (tentatively assigned chirality; | |
| trivial name: fusaminic acid) | |
| 5-Acetamidino-4-O-acetyl- | |
| 3,5,9-trideoxy-L-glycero-L-gluco-non-2- | |
| ulosonic acid (tentatively assigned chirality) | |
| 5-Acetamidino-7-acetamido-3,5,7,9- | |
| tetradeoxy-D-glycero-L-gluco- | |
| non-2-ulosonic acid | |
| (tentatively assigned chirality) | |

Sialic acids may be terminal or internal residues of a glycan. Most commonly, sialic acids are terminal residues of cell surface glycans. As such, sialic acids help to regulate various biological processes including cell-cell signaling, tumor growth and metathesis, cell adhesion, and cellular recognition. Additionally, viruses, such as adeno-assisted viruses can bind to cell surface sialyated glycans (a glycan that includes at least one sialic acid saccharide) and achieve entry into a host cell. Furthermore, lectins may bind to sialyated glycans on biomolecules to initiate downstream biological processes.

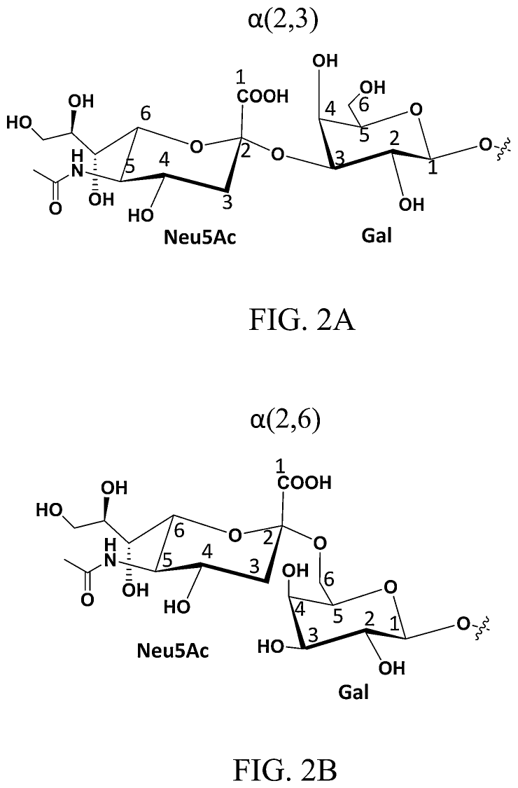

The identity of the sialic acid, the identity of the one or more adjacent saccharides, and the configuration of the glycosidic linkage linking sialic acid to one or more saccharides in a glycan may vary. For example, some cell surface sialyated glycans possess a terminal sialic acid-galactose motif, more specifically an N-acetylneuraminic acid (Neu5Ac)-galactose (Gal) motif (Nue5Ac-Gal). There are various configurations of the glycosidic bond linking the sialic acid and the galactose. The two most common configurations are a alpha(2,3) linkage and an alpha (2,6)linkage. FIG. 2A shows the alpha(2,3) Nue5Ac-Gal linkage and FIG. 2B shows the alpha(2,6) Nue5 Ac-Gal linkage.

In embodiments where the affinity group includes a carbohydrate, the affinity group of the separation ligand may include any monosaccharide, disaccharide, or glycan. In embodiments where the affinity group includes a glycan, the glycan may include any glycosylation pattern; that is, any pattern of monosaccharides, each monosaccharide linked to at least one other monosaccharide through a glycosidic linkage. The monosaccharides included in a glycan affinity group may be any monosaccharide as described herein including stereoisomers thereof, and ionized versions thereof, as well as have any substituent or modification as described herein. Additionally, each glycosidic linkage in a glycan affinity group may be of any configuration as described herein.

The identity of the carbohydrate of the affinity group can be chosen based on the target molecule to be isolated. In some embodiments, the affinity group includes a sialic acid or sialic acid-like monosaccharide. In some such embodiments, the sialic acid or sialic acid-like monosaccharide is a part of a glycan. In some embodiments, the sialic acid or sialic acid-like monosaccharide is the terminal residue of the glycan. In some embodiments, the sialic acid is a terminal Nue5Ac residue that has an α-2,6 glycosidic linkage to an adjacent saccharide. In some such embodiments, the target molecule includes a hemagglutinin (HA) protein (e.g., an HA vaccine). In some embodiments, the sialic acid is a terminal Nue5Ac residue that has an α-2,3 glycosidic linkage to an adjacent saccharide. In some embodiments, the glycan includes a terminal Nue5Ac-Gal motif. In some such embodiments, the terminal Nue5Ac-Gal is an alpha(2,3) Nue5Ac-Gal motif (see FIG. 2A). In other such embodiments, the terminal Nue5Ac-Gal is an alpha(2,6) Nue5Ac-Gal motif (see FIG. 2B). In embodiments when the affinity group includes an alpha(2,3) Nue5Ac-Gal motif or an alpha(2,6) Nue5Ac-Gal motif, the target molecule is a virus (e.g., an adeno-associated virus, an adenovirus, or a lentivirus), or a protein (e.g., a lectin or a recombinant protein that includes a carbohydrate recognizing domain).

In embodiments, where the carbohydrate includes an Nue5Ac residue that has an α-2,6 glycosidic linkage to an adjacent saccharide, the affinity group is the carbohydrate containing protein fetuin or mucin or a fragment thereof. HA purification via fetuin affinity resin chromatography have been presented in the literature. It these cases, fetuin was immobilized to an activated resin and HA was eluted using high conductivity at elevated temperature 37° C. Both fetuin and mucin have been used to in resin chromatography to purify sialic acid-recognizing lectins on the basis of the affinity interaction. Resin-immobilized fetuin chromatography media is commercially available as Fetuin-Agarose (Sigma-Aldrich), containing ≥0.2 mg sialic acid per mL resin. In addition to the slow processing speed typical of resins, this product has another major drawback in that the ligand is attached to matrix by cyanogen bromide linking chemistry, which is unstable and likely to result in constant ligand leakage, limiting it to research use.

In some embodiments, the fetuin if from fetal bovine serum. In some embodiments, mucin is from bovine submaxillary glands. Bovine fetuin and bovine mucin have 5% to 8% and 9% to 24% sialic acid (Nue5Ac) content respectively. Mucin can form multimers between cysteine-rich segments via disulfide bonds. As such, in some embodiments, mucin is used as a separation ligand in the presence of a disulfide reducing agent (e.g., dithiothreitol (DTT)).

In some embodiments, the affinity group does not include fetuin.

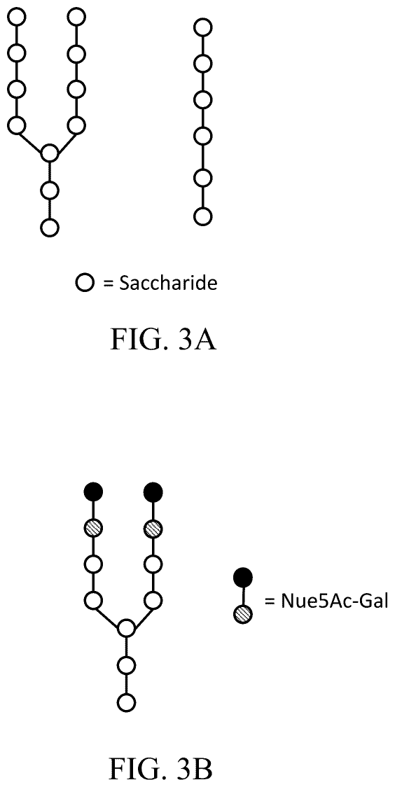

The affinity group may include one or more (e.g., 2, 3, 4, 5, 6, etc.) monosaccharides, disaccharides, or glycans. The affinity group may include a linear or branched glycan. FIG. 3A shows examples of glycan affinity groups that have a linear or branched structure. The linear or branched glycan may include one or more of specific glycosylation patterns that are bound by a given carbohydrate binding domain. For example, an affinity group may include a branched glycan that includes two terminal Nue5Ac-Gal moieties (see FIG. 3B).

In some embodiments, the affinity group is a small molecule that is bound by the target molecule. An example of such a small molecule is 4-mercaptophenyl boronic acid (MPBA).

In some embodiments, the separation media includes a plurality of separation ligands that include a separation group that is an assistance group. An assistance group is a chemical moiety that facilitates the binding of the target molecule to the affinity group; binds the target molecule through electrostatic interactions and/or hydrophobic interactions; or both. In some embodiments, the assistance group may allow for a high density of target molecules to bind to separation ligands that include an affinity group. In some embodiments, the assistance group may aid in attracting the target molecule to the support substrate such as to allow for the target molecule to be in proximity to a separation group that includes an affinity group. For example, the assistance group may be ionizable or possesses a formal charge which may be opposite the charge of the target molecule. In such cases, the oppositely charged assistance group may attract the target molecule to the support substrate which may allow the target molecule to bind to the affinity group.

In some embodiments, the assistance group functions as a cation or anion exchange chromatography ligand. Anion exchange ligands have a positively charged functional group that targets negatively charged target molecules through electrostatic interactions. The anion exchange ligand may possess a formal positive charge, or the positive charge can be induced through the pH of the solution that the anion exchange ligand is exposed to. Cation exchange ligands have a positively charged functional groups that target negatively charged target molecules through electrostatic interactions. The anion exchange ligand may possess a formal negative charge, or the negative charge can be induced through the pH of the solution that the anion exchange ligand is exposed to.

In some embodiments, the assistance group possesses a positive formal charge or is ionizable under certain pH conditions to have a positive charge. Such assistance groups may be beneficial when the target molecule has a negative formal charge. Examples of such assistance ligands include primary, secondary, tertiary, and quaternary amines. Suitable amines may be diamines, triamines, and polyamines.

Examples of primary amines include methylene diamine, ethylene diamine, propylene diamine, butylenediamine (putrescine), pentylamine, or any aliphatic diamine with 1-18 carbons between the terminal amines, covalently attached via one of the amines. Such ligands can be made from polyamines such as ethylene diamine, diethylenetriamine, triethylenetetramine covalently attached via one of the amines.

Examples of secondary amines can include any of the aforementioned primary amines immobilized to the substrate, substituted with an additional R-group as described above. In cases in which diamines are used, secondary amines may also be formed by covalent interaction with the substrate coupling both amines to the substrate. Ligands containing secondary amines with the structure of the ligand may also be immobilized such as linear polyethyleneimine, spermidine, or spermine. Furthermore, groups containing a non-terminal primary amine (e.g., 3-aminopentane) may also be conjugated to the substrate to result in a secondary amine.

Examples of suitable tertiary amines include N,N-dimethylethylenediamine; N,N-dimethylpropylenediamine; N,N-diethylpropylenediamine; or any aliphatic diamine with aliphatic carbon group substitution on one or both amines ranging from one to six carbons, with and a linker having 2-18 carbons between the terminal amines.

Examples of quaternary amines include any of the aforementioned primary amines that have undergone a quaternarization reaction resulting in a permanent positive charge. Such reactions can be performed with alkyl groups such as methyl iodide or aryl groups such as benzyl iodide. Quaternary amines can further include any of the aforementioned tertiary amines that have undergone a quaternarization reaction resulting in a permanent positive charge. Such reactions can be described by the Menshutkin reaction which uses an alkyl halide to form a quaternary ammonium salt from a reaction with a tertiary amine. Such reactions can be performed with alkyl containing groups of varying length such as butyl bromide or aryl groups such as benzyl chloride or combinations therein. Additionally, compounds containing quaternary amines can be immobilized directly.

In other embodiments, the assistance group possesses a negative formal charge or is ionizable under certain pH conditions to have a negative charge. Such assistance group may be beneficial when the target molecule has a positive formal charge. The difference in charge of target molecule and the assistance molecule may allow for an electrostatic interaction between the target molecule and the assistance group thereby allowing the target molecule to be proximate to the support surface and the affinity groups which may increase the probability of the target molecule of binding to an affinity group.

In some embodiments, the assistance group is such that it is able to induce hydrophobic interactions with the target molecules. Hydrophobic interactions exploit the differences in hydrophobicity of between the target molecules and possible impurities. In one embodiment, such ligands include aliphatic chains with three carbons or longer (common used lengths include butyl, pentyl, hexyl, heptyl, octyl, nonyl, decyl, undecyl, and dodecyl), benzyl, phenyl, phenol, pyridine, boronic acid groups, branched polymers such as polypropylene glycol, and sulfur-containing thiophilic ligands such as propanethiol, 2-butanethiol, 3,6-dioxa-1,8-octanedithiol, octanethiol, benzyl mercaptan, 2-mercaptopyridine, thiophenol, 1,2-ethanedithiol, 1,4-benzenedimethanethiol, 2-phenylethanethiol, and the like, and combinations thereof.

In some embodiments, separation ligands that include an assistance group can be directly incorporated into a functionalized layer of a support substrate through polymerization of a monomer that includes an assistance group.

In some embodiments, the separation media includes a plurality of separation ligands that includes a separation group that is a capping group. A capping group is a chemical moiety that prevents reactive groups of the support substrate from reacting with the target molecule or any other molecule in the isolation solution. A capping group may be employed to block support substrate reactive handles that have not reacted with other separation ligands. A capping group may be used to cap the end of a polymer chain. Capping groups may be any chemical group that is non-reactive towards the target molecule or other molecules the isolation solution. In some embodiments, a separation ligand immobilized on a support substrate has the formula (SLim)

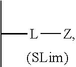

where L is a linker, Z is a separation group, and the vertical black line is the support substrate.

Each separation ligand of the plurality of separation ligands has the formula SL

[symbol]

L-Z (SL)
where L is a linker and Z is a separation group. L separates the support substrate from Z. The separation group may include an affinity group, capping group, or assistance group. The affinity group may be any affinity group as disclosed herein. The capping group may be any capping group as disclosed herein. The assistance group may be any capping group as disclosed herein.

Separation ligands of multiple chemical compositions may be immobilized to a single support substrate. For example, a support substrate may include a first portion of a separation ligands of formula SL and a second portion of separation ligands of formula SL. In some embodiments, the first portion and the second portion of separation ligands include the same affinity group but have different linkers (L). In other embodiments, the first portion and the second portion of the separation ligands may have the same linker but have different separation group.

In some embodiments, L is of formula L1 such that the separation ligand of formula SL is of formula SL1.

Rp¹ (L1)

[symbol]

Rp¹-Z (SL1)

Rp¹ is a reaction product, and Z is the separation group. A reaction product is the chemical group resulting from the reaction of two cooperative functional handles (as discussed herein). In a separation ligand of formula SL1, the linker is the reaction product. The reaction product Rp¹ links the support substrate (not shown) and the separation group (Z). A covalent bond from Rp¹ to the support substrate (M) is the point of covalent attachment of the linker (L1) to the support substrate. A covalent bond from Rp¹ to the separation group (Z) is the point of covalent attachment of the linker (L1) to the separation group (Z). Rp¹ may be any reaction product as disclosed herein.

The reaction product (Rp¹) may be the reaction product between any two cooperative reactive handles (as described herein). Examples of reaction products include amides, ureas, thioureas, carbamates, carbonates, esters, thioethers, ethers, and triazoles. In some embodiments, a reaction product (e.g., such as Rp¹) is Rp^{A}, Rp^{B}, Rp^{C}, Rp^{D}, Rp^{E}, Rp^{F}, Rp^{G}, Rp^{H}, Rp^{I}, Rp^{J}, Rp^{K}, or an isomer thereof. Chemical structures of Rp^{A}-Rp^{K} are depicted below.

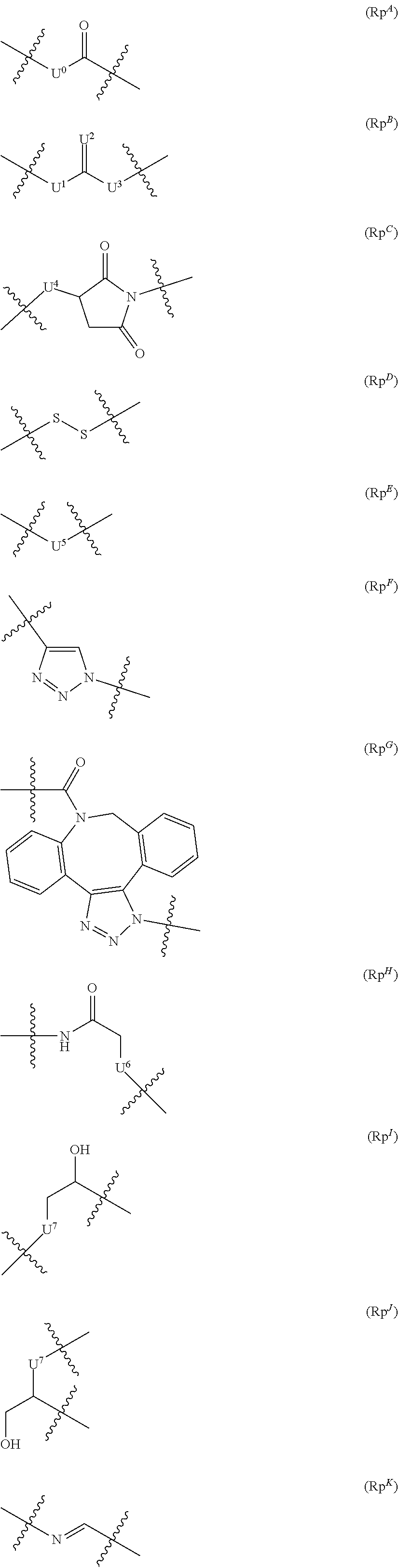

U⁰, U⁴, U⁵, U⁶, and U⁷ are each independently NH, N, O, or S. For Rp^{B} each U1, U2, and U³ are independently NH, N, O, or S. The reaction products have two connection points, each of which may be covalently linked to the support substrate or any component of a separation ligand. For separation ligands of formula SL1 one connection point the reaction product Rp¹ is linked to the separation group while the other connection point of the reaction product Rp¹ is linked to the support substrate.

In some embodiments where the separation ligand is of formula SL1, Rp¹ is Rp^{A} where U⁰ is NH. In some such embodiments, the amide nitrogen (U⁰) of Rp^{A} is covalently linked to the separation group. In other such embodiments, the amide nitrogen of Rp^{A} is covalently linked to the support substrate.

In some embodiments where the separation ligand is of formula SL1, Rp¹ is Rp^{A} where U⁰ is O. In some such embodiments, the ester oxygen (U⁰) of Rp^{A} is covalently linked to the separation group. In other such embodiments, the ester oxygen of Rp^{A} is covalently linked to the support substrate.

The identity of a reaction product (e.g., Rp¹) depends at least in part on the type of conjugation chemistry used to form the reaction product. In a conjugation reaction, each component being linked together includes a reactive handle, such that the reactive handles are cooperative reactive handles. Components that include a reactive handle for conjugation reactions are termed precursor compounds or precursors. A precursor compound includes the component and a reactive handle covalently linked to the component. Cooperative handles or cooperative reactive handles are two or more reactive handles that when exposed to each other under favorable reaction conditions a conjugation reaction occurs to form a reaction product between the reactive handles. Components that have been conjugated through a conjugation reaction may be referred to as a conjugate. For example, component A and component B are to be conjugated through a conjugation reaction. The component A precursor includes a reactive handle X. The component B precursor includes a reactive handle Y. X and Y are cooperative. A conjugation reaction between the component A precursor and the component B precursor results in the formation of an A-B conjugate that includes the reaction product between X and Y. It is understood that the notation of a conjugate is from the perspective of the conjugated components, not the precursors of those components (i.e., A-B conjugate not A precursor-B precursor conjugate). This is because upon completion of the conjugation reaction, the precursor components are no longer precursors. In the case of a component precursor that includes two independently reactive handles, one of which has been reacted with a different component precursor to form a conjugate, the conjugate notation is still from the perspective of the conjugated components, not the precursor components, with the understanding that the conjugate includes the unreacted second reactive handle. For example, a component D precursor includes a first reactive handle J and a second reactive handle Z. The component B precursor has the reactive handle Y. J and Y are cooperative handles. A conjugation reaction between the component A precursor and the component B precursor results in the formation of an A-B conjugate that includes the reaction product between J and Y. The A-B conjugate also includes the unreacted second reactive handle Z.

Any pair of cooperative reactive handles may be used to forma reaction product of the present disclosure. Examples of cooperative handles include an activated ester and an amine; an amine and an NHS-ester; a hydroxyl and an NHS-ester; a hydroxyl and an epoxide; an acyl chloride and an amine; an acyl chloride and an alcohol; an amine and an epoxide; a thiol and an epoxide; a thiol and a maleimide; a disulfide and a thiol; an azide and an alkyne (azide and a linear alkyne in the presence of Cu(I); an azide and a cyclic alkyne such as cyclooctyne, difluorinated cyclooctyne, dibenzocyclooctyne, TMTH-SulfoxImine, biarylazacyclooctynone, or bicyclo[6.1.0]nonyne); an amine and an isocyanate; an amine and an isothiocyanate, a amine and a benzoyl fluoride; a thiol and a iodoacetamide; a thiol and a bromoacetamide; a disulfide and 2-thiopyridine; a thiol and 3-arylpropiolonitirle; a phenol and a diazonium salt; a phenol and 4-phenyl-1,2,4-triazoline-3,5-dione; a phenol and aldehyde, and a aniline; a hydroxyl and sodium periodate; a thiol and an iodoacetamide; an amine and a pyridoxal phosphate; an azide and a functionalized triphenyl phosphine; a tetrazine and a strained alkene; and the like.

Examples of individual reactive handles that may be used to form the separation media of the present disclosure include Rh^{A} (hydroxyl), Rh^{B} (thiol), Rh^{C} (amine), Rh^{D} (activated ester), Rh^{E} (azide), Rh^{F} (alkyne), Rh^{G} (NHS-ester), Rh^{H} (maleimide), Rh^{I} (where X is a Cl, Br, or I leaving group attached to carbon that can undergo nucleophilic substitution; e.g., a bromoacetamide or iodoacetamide), Rh^{J} (cyclooctyne), Rh^{K} (isocyanate), Rh^{L} (isothiocyanate), Rh^{M} (where X is a Cl, Br, or I leaving group attached to carbon that can undergo nucleophilic substitution), Rh^{N} (an epoxide), Rh^{O} (an acyl chloride), Rh^{P} (aldehyde), and isomers thereof. Chemical structures of Rh^{A}-Rh^{P} are depicted below.

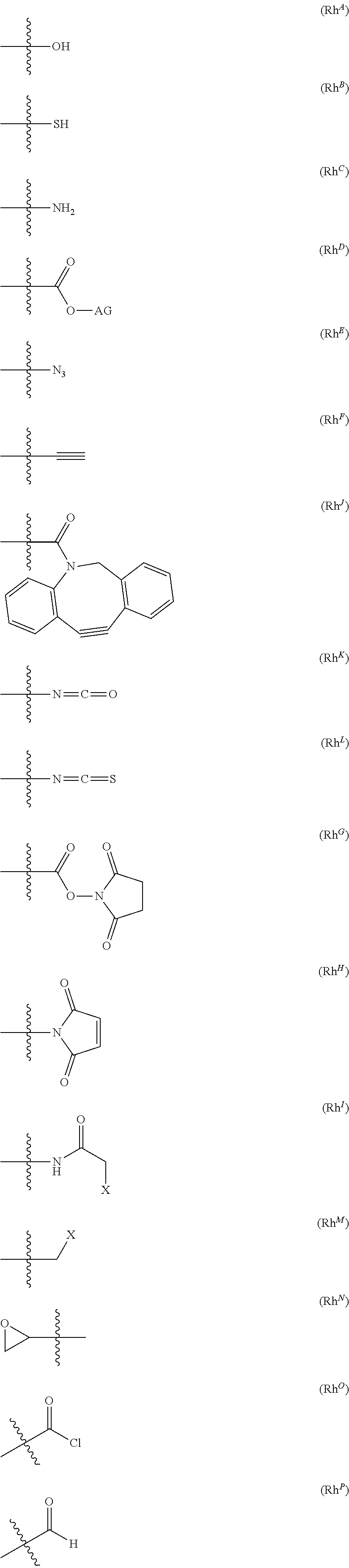

where X in Rh^{M} and Rh^{I} may be -chloro, -bromo, or -iodo.

Rh^{D} is an activated ester where AG is an activating group. An activated ester is an ester that is reactive with an activated ester cooperative reaction handle (e.g., an amide) in a conjugation reaction. Activated esters may be denoted as the type of activated ester or by the activating group. Examples of activating groups include O-acylisoureas, benzotriazoles (with a bond between the ester oxygen and one nitrogen of the triazole), and pentafluorophenyl. In some embodiments, Rh^{D} may be an activated ester of a carboxylic acid. In such embodiments, the activated ester is formed through the reaction of a carboxylic acid with one or more reagents that install the activating group. For example, a carboxylic acid may be converted into an activated ester having a O-acylisoureas activating group by treating the carboxylic acid with various carbodiimide reagents (e.g., N,N′-Dicyclohexylcarbodiimide, 1-Ethyl-3-(3 dimethylaminopropyl)carbodiimide, diisopropylcarbodiimide (DIC) under favorable reaction conditions. A carboxylic acid may be converted into an activated ester having a benzotriazole activating group by treating the carboxylic acid with various carbodiimide reagents followed by treatment with hydroxybenzotriazole (HOBT) or by treating the carboxylic acid with various benzotriazole containing compounds (e.g., O-(Benzotriazol-1-yl)-N,N,N′,N′-tetramethyluronium hexafluorophosphate (HBTU); O-(Benzotriazol-1-yl)- N,N,N′,N′-tetramethyluronium tetrafluoroborate (TBTU); 2-(1H-benzotriazol-1-yl)-1,1,3,3-tetramethyluronium hexafluorophosphate (HBTU); Benzotriazol-1-yloxy)tris(dimethylamino)phosphonium hexafluorophosphate (BOP): (Benzotriazol-1-yloxy)tripyrrolidinophosphonium hexafluorophosphate (PyBOP); and O-(7-Azabenzotriazol-1-yl)- N,N,N′,N′-tetramethyluronium tetrafluoroborate (TATU) under favorable reaction conditions. Other reagents are available for making activated esters from carboxylic acids including bromotripyrrolidinophosphonium hexafluorophosphate (PyBrOP); O-(N-Suc-cinimidyl)-1,1,3,3-tetramethyl-uronium tetrafluoroborate (TSTU); O-(5-Norbornene-2,3-dicarboximido)-N.N.N′,N′-tetramethyluronium tetrafluoroborate (TNTU); O-(1,2-Dihydro-2-oxo-1-pyridyl-N,N,N′,N′-tetramethyluronium tetrafluoroborate (TPTU); and 3-(Diethylphosphoryloxy)-1,2,3-benzotriazin-4(3H)-one (DEPBT); carbonyldiimidazole. In some embodiments, the activated ester may be created in situ from a carboxylic acid and not isolated prior to a conjugation reaction.

Rh^{O} is an acyl chloride. Acyl chlorides may be prepared from carboxylic acids, for example, using thionyl chloride. Acyl chlorides may not be stable and as such, may be prepared in situ and not isolated prior to a conjugation reaction.

Reactive handles Rh^{A}, Rh^{B}, Rh^{C}, Rh^{D}, Rh^{E}, Rh^{F}, Rh^{G}, Rh^{H}, Rh^{I}, Rh^{J}, Rh^{K}, Rh^{L}, Rh^{M}, Rh^{N}, Rh^{O}, and Rh^{P} include various pairs of cooperative handles that can from the reaction products of Rp^{A}, Rp^{B}, Rp^{C}, Rp^{D}, Rp^{E}, Rp^{F}, Rp^{G}, Rp^{H}, Rp^{I}, Rp^{J}, Rp^{K}. For example, under favorable reaction conditions, a conjugation reaction between Rh^{A} and Rh^{D} forms Rp^{A} where U⁰ is O. Under favorable reaction conditions, a conjugation reaction between Rh^{D} and Rh^{C} forms Rp^{A} where U⁰ is NH. Under favorable reaction conditions, a conjugation reaction between Rh^{C} and Rh^{G} forms Rp^{A} where U⁰ is NH. Under favorable reaction conditions, a conjugation reaction between Rh^{B} and Rh^{H} forms Rp^{C} where U⁴ is S. Under favorable reaction conditions, a conjugation reaction between two Rh^{B} forms Rp^{D}. Under favorable reaction conditions, a conjugation reaction between Rh^{C} and Rh^{I} forms Rp^{H} where U⁶ is NH. Under favorable reaction conditions, a conjugation reaction between Rh^{B} and Rh^{I} forms Rp^{H} where U⁶ is S. Under favorable reaction conditions, a conjugation reaction between Rh^{M} and Rh^{B} forms Rp^{E} where U⁵ is S. Under favorable reaction conditions, a conjugation reaction between Rh^{M} and Rh^{C} forms Rp^{E} where U⁵ is NH. Under favorable reaction conditions, a conjugation reaction between Rh^{K} and Rh^{C} forms Rp^{B} where U¹ and U³ are NH and U² is O. Under favorable reaction conditions, a conjugation reaction between Rh^{L} and Rh^{C} forms Rp^{B} where U¹ and U³ are NH and U² is S. Under favorable reaction conditions, a conjugation reaction between Rh^{F} and Rh^{E} forms Rp^{F}. Under favorable reaction conditions, a conjugation reaction between Rh^{J} and Rh^{E} forms Rp^{G}. Under favorable reaction conditions, a conjugation reaction between Rh^{N} and Rh^{A} forms Rp^{I} or Rp^{J} where U⁷ is O. Under favorable reaction conditions, a conjugation reaction between Rh^{N} and Rh^{B} forms Rp^{I} or Rp^{J} where U⁷ is S. Under favorable reaction conditions, a conjugation reaction between Rh^{N} and Rh^{C} forms Rp^{I} or Rp^{J} where U⁷ is N. Under favorable reaction conditions, a conjugation reaction between Rh^{O} and Rh^{A} forms Rp^{A} where U⁰ is O. Under favorable reaction conditions, a conjugation reaction between Rh^{O} and Rh^{B} forms Rp^{A} where U⁰ is NH. Under favorable reaction conditions, a conjugation reaction between Rh^{P} and Rh^{C} forms Rp^{K}.

Conjugation reactions between cooperative handles may be done under favorable reaction conditions. Favorable reaction conditions are conditions that facilitate a reaction, increase the yield of a reaction, minimize unwanted biproducts of a reaction, and/or increase the rate of a reaction. Example reaction conditions include reaction temperature, reaction atmosphere composition, reaction solvent, the presence of a catalyst, the presence of a base, the presence of an acid, and combinations thereof. Favorable reaction conditions for conjugation reactions are known.

Cooperative handles may be chosen such that the conjugation reaction is an orthogonal conjugation reaction. Orthogonal conjugation reactions are reactions where the chemistry is selective such that only two cooperative handles react to form a reaction product even when additional reactive handles or pairs of cooperative reactive handles may be present. Orthogonal conjugation reactions may be useful because they allow for multiple selective conjugation reactions to take place in series or in parallel. Orthogonality of two or more conjugation reactions may be achieved by choosing reactive handles that are only reactive with their cooperative counterpart in the presence of other cooperative reactive handle pairs. Orthogonality of two or more conjugation reactions may also be achieved by using reactive handles that are reactive with multiple cooperative counterparts, but the reactivity can be influenced through the reaction conditions such that only a specific pair of cooperative handles will react in the given set of reaction conditions.

To form a separation ligand of formula SL1, conjugation reaction precursor compounds are employed, each precursor compound having a reactive handle that is cooperative with the reactive handle of a different precursor compound. In some embodiments, a separation ligand of formula SL1 is formed through the conjugation of a separation group precursor of formula Pre-Z(1) and a support substrate precursor of formula Pre-M(1) by way of synthetic scheme S1. The support substrate precursor includes a reactive handle Rh1 that is covalently attached to the support substrate (thick black vertical line). The separation group precursor includes the separation group (Z) of formula SL1 and a separation group reactive handle Rh². Rh¹ and Rh² are cooperative reactive handles and may be any pair of cooperative handles as disclosed herein. In scheme S1, the support substrate reactive handle (Rh¹) is reacted with the separation group reactive handle (Rh²) to from a reaction product (Rp¹) thereby forming a separation ligand of formula SL1.

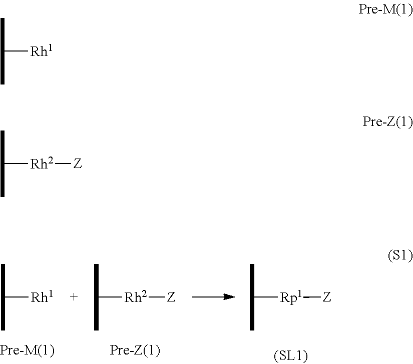

In some embodiments the material of the support substrate does not include a reactive handle that is cooperative with the separation group reactive handle (Rh²). In such embodiments, scheme S1 may further include installing the support substrate reactive handle Rh¹. The support substrate reactive handle Rh¹ may be installed through treatment of the support substrate to form the Rh¹. In such embodiments, a chemical functionality already present on the support substrate is transformed into the support substrate reactive handle. For example, the support substrate may be exposed to an oxidizing or reducing reagent (or conditions). The support substrate reactive handle Rh¹ may be installed through the installation of a functionalized layer. In such embodiments, the functionalized layer is considered a part of the support substrate. In such embodiments, the reactive handle of functionalized layer is the support substrate reactive handle. Examples of materials suitable for a functionalized layer are discussed herein.

In embodiments, where the separation group includes carbohydrate containing protein, the separation group reactive handle may be the side chain of an amino acid. For example, in some embodiments, the separation group reactive handle is the amine of the side chain of lysine. In some embodiments, the separation group reactive handle is the hydroxyl side chain of the amino acid serine or threonine. In some embodiments, the separation group reactive handle is the thiol of the amino acid side chain of cysteine. Because proteins may have multiple amino acids of the same type, it may be difficult to control the location of the reactive handle on the affinity group. For this reason, in some embodiments, the plurality of separation groups having an affinity group may have some affinity groups attached to the support substrate at one reactive handle location and other affinity groups attached to the support substrate at a different reactive handle location. Additionally, in some embodiments, the plurality of separation groups having an affinity group may have some affinity groups attached to the support substrate with a first reaction product and others attached with a second reaction product.

In some embodiments, the carbohydrate containing protein may be engineered to include an amino acid residue that has a reactive handle at a specific location on the protein (e.g., near the C or N terminus). In some such embodiments, the amino acid residue is a natural amino acid that has a side chain with a reactive handle (e.g., lysine, serine, threonine, cysteine). In other embodiments, the amino acid residue is an unnatural amino acid that has a side chain that includes a reactive handle. Examples of unnatural amino acids that have side chains with reactive handles include those that include an azide (e.g., 3-azido-alanine, 6-azido lysine, 4 azido phenylamine, (2S,4S)-Fmoc-4-azido-pyrrolidine-2-carboxylic acid, 2-(R)-Fmoc-amino-3-azidopropionic acid, and 4-(4-Azidophenyl)butyric acid) and those that include and alkyne (e.g., L-Homopropargylglycine). In some embodiments, where affinity group is engineered to include a reactive handle, the reactive handle may be separated from the affinity group by a linker. The linker may be an amino acid sequence.

In some embodiments, the linker of formula LI of formula SL is derived from an amino acid or a peptide. In some embodiments, the linker of formula LI is designed to mimic N-glycosidic linkages of glycans to an asparagine residue or peptides and/or proteins found in nature. In some such embodiments, the separation ligand may be of formula X, XI, XIII, or XIII.

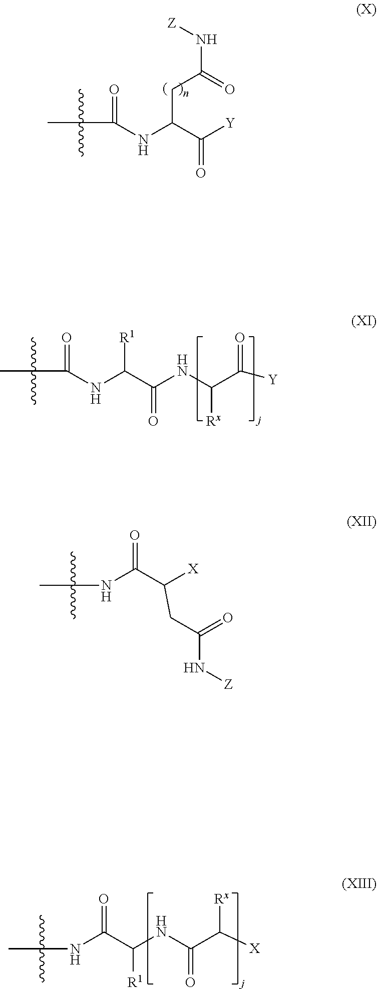

where Z is the separation group; R¹ is an amino acid side chain or a protected amino acid side chain; X is NH₂ or PG_{N} where PG_{N} is an amine protecting group; Y is OH or a PG_{C(O)OH} where PG_{C(O)OH} is a carboxylic acid protecting group; each R^{X} is an amino acid side chain, a protected amino acid side chain, or

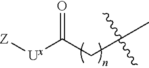

where at least one R^{X} is

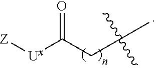

Each U^{X} is NH or O. j is 1, 2, 3, 4, 5, 6, 7, 8, 9, or 10. Formula X, XI, XII, and XIII all have a first reaction product Rp¹ that is Rp^{A} where U⁰ is NH.

R¹ and each R^{X} may be any amino acid side chain. An amino acid side chain is the chemical group extending from the alpha carbon of the amino acid. In some embodiments, R¹ and each R^{X} may independently be the amino acid side chain of arginine, histidine, lysine, aspartic acid, glutamic acid, serine, threonine, asparagine, glutamine, cysteine, selenocysteine, alanine, valine, isoleucine, leucine, methionine, phenylalanine, tyrosine, tryptophane, or an unnatural amino acid. The amino acid side chain may be a protected amino acid side chain; that is, the amino acid side chain may include a protecting group that masks a reactive group. For example, the amine of the side chain of lysine may be protected with an amine protecting group (e.g., tert-butyloxycarbonyl; allyloxycarbonyl; and benzyloxycarbonyl). The carboxylic acid of the side chain of aspartic acid and glutamic acid may be protected with a carboxylic acid protecting group (e.g., methyl ester; tert-butyl ester; 2,4-dimethyoxybenzyl ester; 9-fluorenylmethyl ester; and benzyl ester). The guanidinium of the side chain of arginine may be protected with a guanidinium protecting group (e.g., 2,2,4,6,7,-pentamethyl-2,3-dihydrobenzofuran-5-sulfonyl). The amide of the side chain of asparagine and glutamine may be protected with an amide protecting group (e.g., 9-xanthenyl). The thiol of the side chain of cysteine may include a thiol protecting group (e.g., trityl, p-methylbenzyl and acetamidomethyl). The side chain of serine or threonine may include an alcohol protecting group (e.g., tert-butyldimethylsilyl; allyl; and o-nitrobenzyl).

In some embodiments, X is an amine protecting group (PG_{N}). In some embodiments, Y is a carboxylic acid protecting group (PG_{C(O)OH)}. PG_{N} and PG_{C(O)OH} may be any amine and carboxylic acid protecting group, respectively, as described herein.

In some embodiments, a separation ligand of formula X, XI, XII, or XIII can be formed from a single conjugation reaction; that is, by way of synthetic scheme 1 (S1). More specifically, separation ligand of formula X can be formed by way of synthetic scheme S1(a); a separation ligand of formula XII can be formed by way of synthetic scheme S1(b); a separation ligand of formula XII can be formed by way of synthetic scheme S1(c); and a separation ligand of formula XIII can be formed by way of synthetic scheme S1(d) where the formula for the support substrate precursor (Pre-M(1a) and Pre-M(1b)) and the separation group precursor (Pre-Z(1a), Pre-Z(1b), Pre-Z(1c), and Pre-Z(1d)) are shown for each respective synthetic scheme.

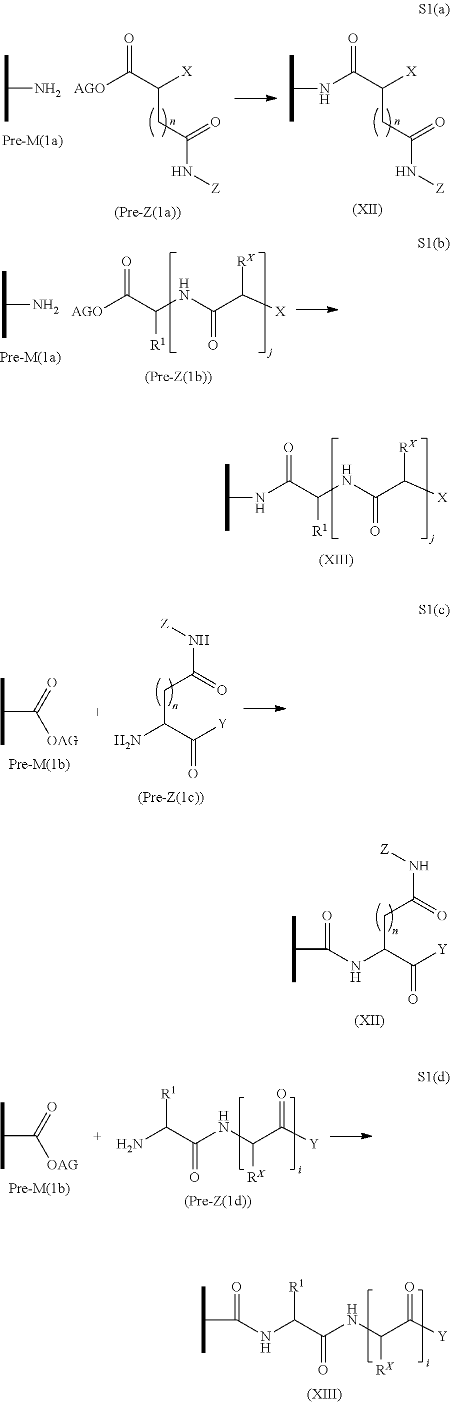

In scheme S1(a) and S1(b), the support substrate precursor (Pre-M(1a)) includes an amine reactive handle (i.e., the support substrate reactive handle). The counterpart separation group precursors include an activated ester reactive handle (i.e., the separation group reactive handle). The amine reactive handle is reacted with the activated ester reactive handle to form an amide reaction product (i.e., Rp¹ of scheme S1).

In scheme S1(c) and S1(d), the support substrate precursor (Pre-M(1b)) includes an activated ester reactive handle (i.e., the support substrate reactive handle). The counterpart separation group precursors include an amine reactive handle (i.e., the separation group reactive handle). The amine reactive handle is reacted with the activated ester reactive handle to form an amide reaction product (i.e., Rp¹ of scheme S1).

The conjugation reactions of S1(a), S1(b), S1(c), and S1(d) may be conducted similar to solid phase peptide coupling chemistry. In some such embodiments, the scheme further includes preparing the activated ester by reacting the carboxylic acid with an activating reagent to form the activated ester. The activated ester may be prepared from a carboxylic acid using reagents as described elsewhere herein. The activated ester is then reacted with the amine to form an amide bond (e.g., a peptide bond). Following the conjugation, any amino acid, carboxylic acid, or amine protecting groups can be deprotected if desired.

In some embodiments, the amine reactive handle of the separation group precursor (e.g., Pre-Z(1c) or Pre-Z(1d)) may be protected by an amine protecting group. In such embodiments, the synthetic scheme may first include deprotecting the protected amine to expose the amine reactive handle (i.e., the separation group reactive handle).

In some embodiments, the carboxylic acid from which the activated ester reactive handle is derived of the separation group precursor (e.g., Pre-Z(1a) or Pre-Z(1b)) may be protected by a carboxylic acid protecting group. In such embodiments, the synthetic scheme may first include deprotecting the protected carboxylic acid to expose the carboxylic acid. The scheme may further include reacting a carboxylic acid with a carboxylic acid activating reagent to form the activated ester reactive handle (i.e., the separation group reactive handle).

In some embodiments, the linker (L) is of formula L2 such that SM is of formula SL2; that is:

Rp³-Sp-Rp⁴ (L2)

[symbol]

Rp³-Sp-Rp⁴-Z (SL2)

In formulas L2 and SL2, Sp is a spacer, Rp³ is a first reaction product, and Rp⁴ is a second reaction product. In formula SL2, Z is the separation group. The linker of formula L2 includes Sp, Rp³ and Rp⁴. In a separation ligand of formula SL2, a covalent bond from Rp³ to the support substrate is the point of covalent attachment of the linker (L2) to the support substrate. A covalent bond from Rp+to the separation group (Z) is the point of covalent attachment of the linker (L2) to the separation group (Z). Rp³ and Rp+may be any reaction product as described herein.

In some embodiments where the separation ligand is of formula SL2, Rp³, Rp⁴, or both are Rp^{A} where U⁰ is NH. In some such embodiments, the amide nitrogen (U⁰) of Rp^{A} is covalently linked to the support substrate or covalently linked to the separation group. In other such embodiments, the amide nitrogen of Rp^{A} is covalently linked to the spacer.

In some embodiments where the separation ligand is of formula SL2, Rp³, Rp⁴, or both are Rp^{A} where U⁰ is O. In some such embodiments, the ester oxygen (U⁰) of Rp^{A} is covalently linked to the support substrate or covalently linked to the separation group. In other such embodiments, the ester oxygen of Rp^{A} is covalently linked to the spacer.

The spacer (Sp) of the linker (L1) may be of any length and/or chemical composition that does not completely inhibit the formation of the first reaction product and the second reaction product. The spacer (Sp) may be of any length and/or chemical composition that does not completely inhibit the ability of the affinity group to bind to its intended target.

The spacer (Sp) includes a divalent organic group. The divalent organic group includes a backbone. The “backbone” is the longest contiguous chain of atoms within the spacer (Sp). In some embodiments, the backbone is a carbon-based backbone. A backbone that is carbon-based is a backbone that has a greater number of carbon atoms than heteroatoms in the backbone. The backbone may include one or more substitutions extending from the backbone and/or one or more functional groups catenated within the backbone.

In some embodiments, the backbone is an alkanediyl (divalent group that is a radical of an alkane) or an alkenediyl (divalent group that is a radical of an alkene). The alkanediyl or alkenediyl may have a backbone chain length of C1 to C18, C1 to C10, C1 to C6, C1 to C4, C1 to C3, or C2 to C4. An alkenediyl may have one or more double bonds. The one or more double bonds may be located at any point along the backbone.

In some embodiments, the backbone includes one or more catenated functional groups. Catenated functional groups have at least one atom that is a part of the backbone; that is, at least one atom of the functional group lies within the backbone chain. The at least one atom of the functional group that is a part of the backbone can be a carbon or a heteroatom. For example, in some embodiments, the backbone includes a catenated ketone where the carbon atom of the carbonyl of the ketone is a part of the backbone. In other embodiments, the backbone includes a catenated amide. In some such embodiments, the nitrogen of the catenated amide is a part of the backbone and the carbon of the carbonyl is not a part of the backbone. In other such embodiments, the nitrogen and the carbonyl carbon of the amide are a part of the backbone. Example catenated functional groups include, ethers; thioether; esters (where the ester oxygen atom is a part of the backbone, or where the ester oxygen and the carbonyl carbon are a part of the backbone); thioesters (where the thioester sulfur atom is a part of the backbone, or where the thioester sulfur atom and the carbonyl carbon are a part of the backbone); amides (where the amide nitrogen is a part of the backbone, or where the amide nitrogen and the carbonyl carbon are a part of the backbone); ureas (where one of the urea nitrogens is a part of the backbone, or where both of the urea nitrogens and the carbonyl carbon are a part of the backbone); carbamates (where the carbamate oxygen is a part of the backbone; the carbamate nitrogen is a part of the backbone; or the carbamate oxygen, the carbamate nitrogen, and the carbonyl carbon are a part of the backbone); thioureas (where one of the urea nitrogens is a part of the backbone, or where both of the urea nitrogens and the carbonyl carbon are a part of the backbone); secondary and tertiary amines; aromatic rings (where at least two atoms of the aromatic ring are a part of the backbone); and any combination thereof.

In some embodiments the spacer includes a catenated ether (i.e., a catenated oxygen atoms). In some such embodiments, the backbone includes a polyethylene glycol chain of 1, 2, 3, 4, 5, 6, 7, 8, 9, or 10 —OCH₂CH₂— repeat units.

In some embodiments, the spacer includes a catenated ketone. In some such embodiments, the spacer is of the formula —(CO)—. In some embodiments, the spacer includes a C1 alkenediyl backbone and a catenated carbonyl where the C1 is the carbonyl carbon of the catenated ketone.

In some embodiments where the separation ligand is of formula SL2, Rp³ and Rp⁴ are both Rp^{E} where each U⁵ is independently O, NH, or S. In some embodiments, the U⁵ of Rp³ is O and the U⁵ of Rp⁴ is O. In some embodiments, the U⁵ of Rp³ is NH and the U⁵ of Rp⁴ is NH. In some embodiments, the U⁵ of Rp³ is O and the U⁵ of Rp⁴ is NH. In some embodiments, the U⁵ of Rp³ is NH and the U⁵ of Rp⁴ are O.

In some embodiments where Rp³ and Rp⁴are both Rp^{E}, Sp may be —C(O)—. In some such embodiments, L2 may be described as Rp^{B}. In some embodiments were L2 is Rp^{B}, U² is O. In some embodiments were L2 is Rp^{B}, U¹ is O. In some embodiments were L2 is Rp^{B}, U³ is O. In some embodiments were L2 is Rp^{B}, U¹ is NH. In some embodiments were L2 is Rp^{B}, U³ is NH. In some embodiments were L2 is Rp^{B}, U¹ is O, U² is O, and U³ is NH. In some embodiments were L2 is Rp^{B}, U¹ is NH, U² is O, and U³ is O.

In some embodiments where the separation ligand is of formula SL2, Rp³ is Rp^{E} and Rp⁴ is Rp^{I} or Rp^{J} where U⁵ and U⁷ are each independently O, NH, or S. In some embodiments U⁵ is NH and U⁷ is NH. In some embodiments U⁵ is O and U⁷ is O. In some embodiments U⁵ is NH and U⁷ is O. In some embodiments U⁵ is O and U⁷ is NH.

In some embodiments where Rp³ and Rp⁴ are both Rp^{E}, Sp may be —(CH₂)ₙ- where n is 0, 1, 2, 3, 4, 5, 6, 7, 8, 9, or 10. In some such embodiments, L2 is of the formula

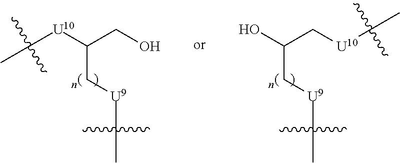

where U⁹ and U¹⁰ are each independently O, NH, or S and n is 0, 1, 2, 3, 4, 5, 6, 7, 8, 9, or 10. U⁹ may be U⁵ from Rp^{I} and U¹⁰ may be U⁷ from Rp^{J}. In some embodiments U⁹ is NH and U¹⁰ is NH. In some embodiments U⁹ is O and U¹⁰ is O. In some embodiments U⁹ is NH and U¹⁰ is O. In some embodiments U⁹ is O and U¹⁰ is NH.

In some embodiments, a separation ligand of formula SL is of formula

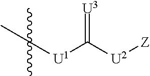

where U¹, U², and U³ are each independently O, NH, or S and Z is a separation group. For example, in some embodiments, a separation ligand of formula SL is

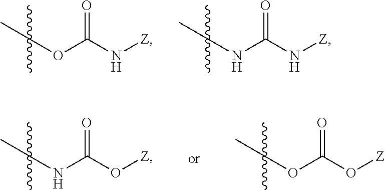

where Z is a separation group. In some embodiments, Z is an affinity group.

In some embodiments, a separation ligand of formula SL is of formula

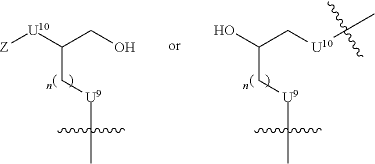

U⁹ and U¹⁰ are each independently O, NH, or S where Z is a separation group. In some embodiments, SL is

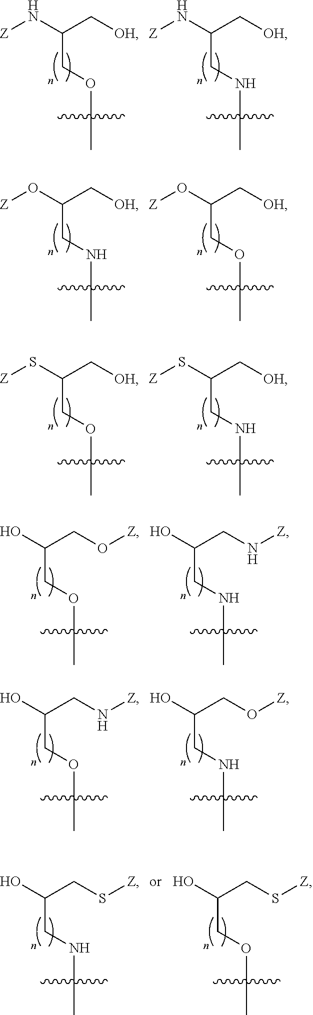

where Z is a separation group and n is 0, 1, 2, 3, 4, 5, 6, 7, 8, 9, or 10. In some embodiments Z is an affinity group.

To form a separation ligand of formula SL2, a series of conjugation reaction precursor compounds are employed, each precursor compound having a reactive handle that is cooperative with the reactive handle of a different precursor compound. In some embodiments, a separation media of formula SL2 is formed through the conjugation of a linker precursor of formula Pre-L, an affinity group precursor of formula Pre-Z(2), and a support substrate precursor of formula Pre-M(2). The linker precursor (Pre-L) includes the spacer Sp of the separation media of formula SL2, a first linker reactive handle Rh³, and a second linker reactive handle Rh⁴. The support substrate precursor (Pre-M(2)) includes a support substrate (vertical black line) and a support substrate reactive handle Rh⁵. The separation group precursor (Pre-Z(2)) includes the separation group Z of formula SL2 and a separation group reactive handle Rh⁶.

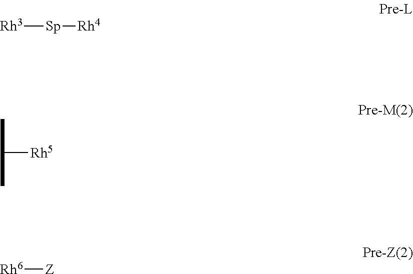

Rh³ and Rh⁵ are a pair of cooperative reactive handles. Rh⁴ and Rh⁶ are a pair of cooperative reactive handles. Rh³ of the linker precursor reacts with Rh⁵ of the support substrate precursor in a conjugation reaction to from a reaction product (i.e., Rp³ of formula SL2). Rh⁴ of the linker precursor reacts with Rh⁶ of the separation group precursor in a conjugation reaction to from a reaction product (i.e., Rp⁴ of formula SL2).

Because the linker precursor includes two reactive handles, the linker precursor is a bifunctional linker. In some embodiments, the linker precursor may be a multifunctional linker precursor that has three or more reactive handles. At least one of the reactive handles is configured to react with the support substrate precursor. The additional reactive handles may be configured to react with a cooperative reactive handle on one or more separation groups. Examples, of bifunctional and multifunctional linker precursors include, epichlorohydrin, diglycidyl ether, triglycidyl ether, tetraglycidyl ether, triazine, poly triazine, poly acrylic (e.g., the COOH groups can be made into activated ester reactive handles), succinic acid (e.g., the COOH groups can be made into activated ester reactive handles), and N′N′-disuccinimidyl carbonate (DSC).

In some embodiments, a separation ligand of formula SL2 may be formed through two conjugation reactions. The reactions may be conducted in any order or simultaneously. For example, in some embodiments, a separation ligand of formula SL2 is formed by way of synthetic scheme 2 (S2).

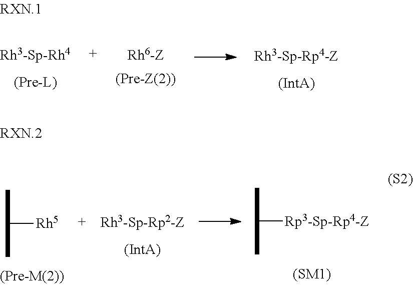

In a first conjugation reaction of scheme S2 (RXN1), the separation group reactive handle (Rh⁶) is reacted with a first linker reactive handle (Rh⁴) in a first conjugation reaction to from a first reaction product Rp⁴ thereby resulting in intermediate A (IntA). Intermediate A is a linker-separation group conjugate that includes the first reaction product (Rp⁴) and the second linker reactive handle (Rh³). IntA may be isolated or taken forward to the second conjugation reaction without isolation. In a second conjugation reaction of S2 (RXN 2) the second linker reactive handle (Rh³) of IntA is reacted with the support substrate reactive handle (Rh⁵) to form a second reaction product (Rp³), thereby forming a separation ligand of formula SL2.

In some embodiments, a separation ligand of formula SL2 is formed by way of synthetic scheme 3 (S3).

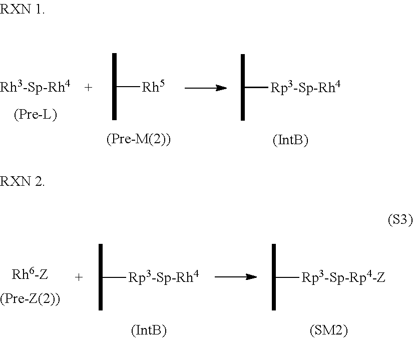

In a first conjugation reaction of scheme S3 (RXN1), the support substrate reactive handle (Rh⁵) is reacted with a first linker reactive handle (Rh³) in a first conjugation reaction to from a first reaction product (Rp³) thereby resulting in intermediate B (IntB). Intermediate B is a linker-support substrate conjugate that includes the first reaction product (Rp³) and the second linker reactive handle (Rh⁴). IntB may be isolated or taken forward to the second conjugation reaction without isolation. In a second conjugation reaction of S3 (RXN 2) the second linker reactive handle (Rh⁴) of IntB is reacted with the separation group reactive handle (Rh⁶) to form a second reaction product (Rp⁴), thereby forming a separation ligand of formula SL2.

Synthetic scheme S4 and synthetic scheme S5 are examples of forming a separation ligand of formula SL2 through scheme S3 using the bifunctional linker (Pre-L) N,N′-disuccinimidyl carbonate (S3) or epichlorohydrin (S4). In both S4 and S5, R¹⁰ can be OH, NH₂, or SH and R¹¹ can be O, NH, or S depending on the identity of R¹⁰.

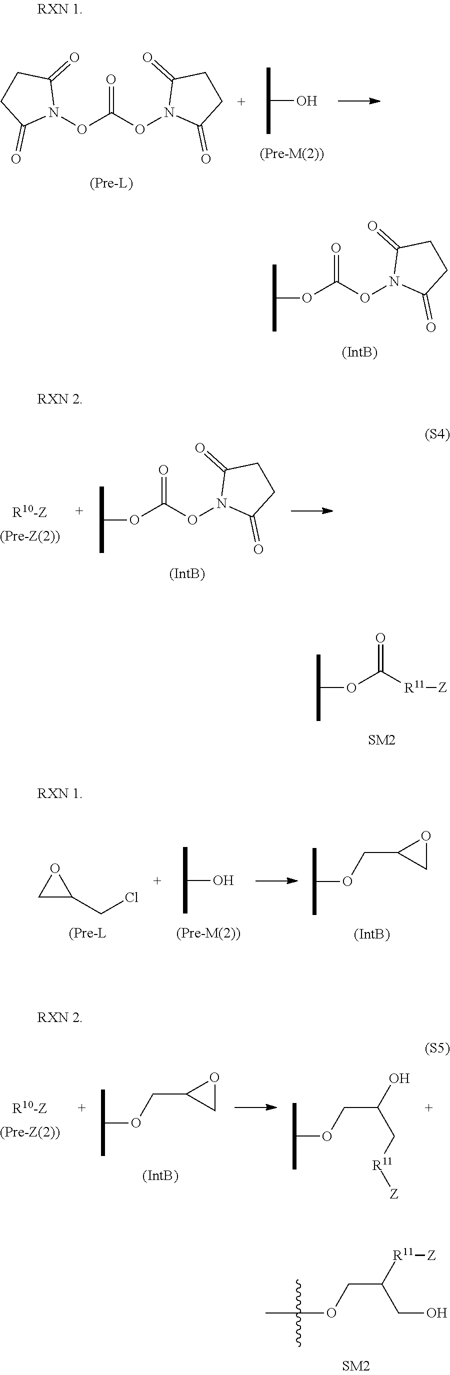

It is noted that separation ligands of formula X, XI, XII, and XIII include both a first reaction product and a second reaction product (Rp^{A} where U⁰ is NH). As such, a separation ligand of any one of formula X, XI, XII, XIII, may be synthesized according to scheme S2 or S3 given the appropriate precursor compounds.

The present disclosure provides methods of making the separation media of the present disclosure. The separation media may be made methods described in PCT application number PCT/US2019/065805 (WO2020123714A1, Zhou), which is incorporated by reference in its entirety.

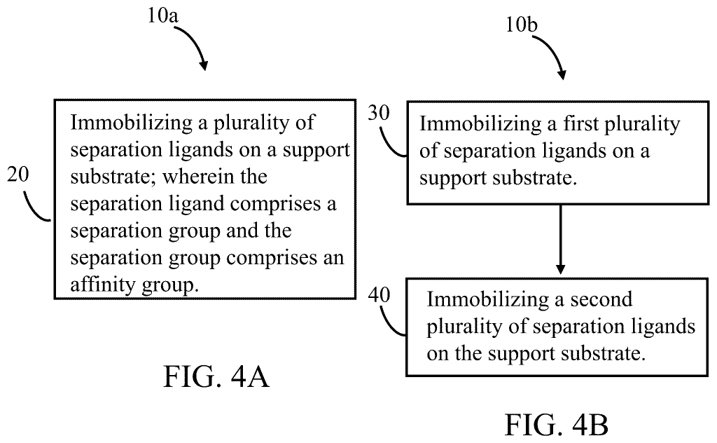

FIG. 4A is a flow diagram depicting a general method 10a for making a separation media of the present disclosure. The general method 10a includes immobilizing a plurality of separation ligands on a support substrate (step 20). Each separation ligand includes a separation group and a linker. The separation group includes the affinity group. Each separation ligand may be of formula SM, SM1, SM2, X, XI, XII, or XIII. Each separation ligand can be immobilized according to any relevant synthetic scheme described herein (e.g., S1, S1(a), S1(b), SI(c), SI(d), S2, S3, S4, or S5).

In some embodiments, the separation media includes a first plurality of separation ligands immobilized on the support substrate and a second plurality of separation ligands immobilized on the support substrate. FIG. 4B is a flow diagram depicting a general method 10b for making a separation media of the present disclosure that includes at least two pluralities of separation ligands. Each plurality of separation ligands immobilized on a support substrate may be of formula SLim. Each separation ligand of the first plurality of separation ligands and the second plurality of separation ligands includes a separation group and a linker. Each separation ligand of the first plurality of separation ligands and the second plurality of separation ligands may be of formula SM, SM1, SM2, X, XI, XII, or XIII. Each separation ligand of the first plurality of separation ligands and the second plurality of separation ligands can be immobilized according to any relevant synthetic scheme described herein (e.g., S1, S1(a), S1(b), S1(c), S1(d), S2, S3, S4, or S5).

In some embodiments of method 10b, the first plurality of separation ligands includes an assistance group, and the second plurality of separation ligands includes an affinity group. Without wishing to be bound by theory, it is thought that the assistance groups of the first plurality of separation ligands can interact with (e.g., via electrostatics and/or hydrophobic or hydrophilic interactions) with the affinity group of the separation group precursor used to form the second plurality of separation ligands. Through these interactions, the second separation group precursors may concentrate on the surface of the support substrate thereby increasing conjugation reaction efficiency (e.g., speed and/or yield). An increase in reaction efficiency may allow a lower concentration of the second plurality of the second separation group precursors to be used in the reaction step than would be needed to achieve the same reaction yield and/or surface coverage without the use of assistance groups. In some embodiments, the assistance group includes an amine. In such embodiments where separation ligands that include an amine assistance group are immobilized prior to immobilization of separation ligands containing affinity groups, the method is amine assisted.

The method 10b includes immobilizing the first plurality of separation ligands on a support substrate (step 30). The method 10b further includes immobilizing the second plurality of separation ligands on the support substrate (step 40).

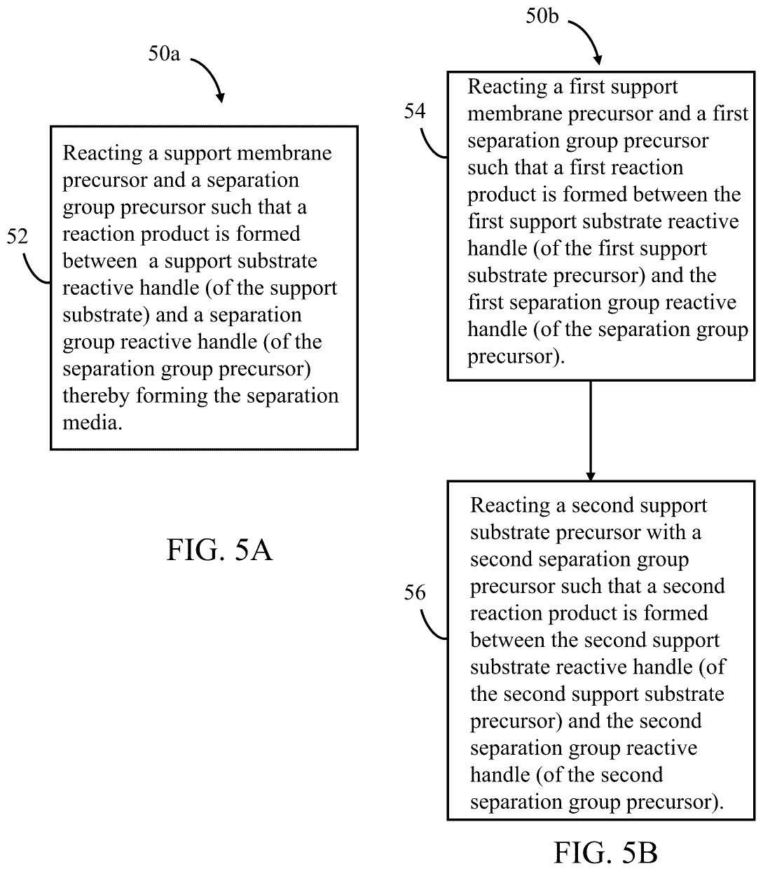

In some embodiments, method 10a or 10b may include method 50a. FIG. 5A is a flow diagram outlining method 50a for making a separation media including a separation ligand of the present disclosure. Method 50a may be understood in reference to synthetic scheme S1 as described herein; however, it is understood that method 50a is not limited to the synthetic scheme S1. The separation ligand of the separation media made from method 50a is synthesized from two components, a separation group precursor (e.g., Pre-Z(1)) and a support substrate precursor (e.g., Pre-M(1)). The separation group precursor includes the separation group (Z) and a separation group reactive handle (Rh¹). The support substrate precursor includes a support substrate (thick vertical black line) and a support substrate reactive handle (Rh²). The separation group reactive handle and the support substrate reactive handle are cooperative handles. Method 50a includes reacting a support substrate precursor and a separation group precursor such that a reaction product (e.g., Rp¹) is formed between the support substrate reactive handle (of the support substrate precursor) and the separation group reactive handle (of the separation group precursor) thereby forming the separation media (e.g., the immobilized separation ligand of Formula SLim).

In some embodiments, step 52 may be accomplished using a reaction mixture. The reaction mixture includes a solvent and the separation group precursor. The reaction mixture may be applied to the support substrate, or the support substrate may be submerged in the reaction mixture. The solvent may include an organic solvent, water, or both. In some embodiments, the solvent is an aqueous buffer that includes one or more salts and/or buffering agents as disclosed herein. The reaction mixture may include additional compounds that facilitate the reaction. For example, the reaction mixture may include an acid, a base, an initiator, a catalyst, or any combination thereof.

In some embodiments where the solvent includes an organic solvent, the reaction step is considered to be “organic assisted” or “organic solvent assisted.” In an organic assisted method, the solvent of the reaction mixture includes water and at least one water-miscible organic solvent. Examples of water-miscible organic solvents include ethanol, acetone, acetonitrile, methanol, propanol (e.g., 2-propanol, 1-propanol), 2-butanol, tetrahydrofuran, dimethylformamide, and dimethyl sulfoxide. The ratio of water to organic solvent in the reaction mixture is such that the reaction mixture is at or near the cloud point of the mixture. The cloud point is the point at which a liquid solution undergoes a liquid-liquid phase separation to from an emulsion or a liquid-solid phase transition to form a stable suspension or a precipitate. The cloud point can be visualized by observing the water-to-organic solvent ratio at which the reaction mixture becomes turbid. Without wishing to be bound by theory, it is thought that including an organic solvent in the reaction mixture such that the reaction mixture is at or near the cloud point increases the conjugation reaction efficiency. The organic solvent molecule can displace water molecules in the separation group precursor thereby increasing interactions between the separation group precursor and the support substrate.

It is possible to define a range of appropriate amounts of organic solvent in the reaction mixture in which the upper boundary is expressed by [V%_{cp}+a(100%−V%_{cp})] and the lower boundary is expressed by [V%_{cp}−bV%_{cp}], where “V%_{cp}” is the percent by volume of the organic solvent in the reaction mixture at the cloud point, “a” is the upper deviation from the cloud point, and “b” is the lower deviation from the cloud point. For the purpose of an example, if the percent by volume of the organic solvent in the ligand solution at the cloud point (V%_{cp}) is 60%, and the upper and lower boundaries are defined by a=0.3 and b=0.5, then the corresponding appropriate amounts of organic solvent in the reaction mixture would range from 30% to 72% organic solvent by volume. In embodiments, the reaction mixture can include an amount of organic solvent in which “a” is about 0.01, 0.02, 0.03, 0.04, 0.05,0.06, 0.07, 0.08, 0.09, 0.1, 0.12, 0.14, 0.16, 0.18, 0.2, 0.25, 0.3, 0.4, 0.5, 0.6, 0.7, 0.8, 0.9, 0.99 and “b” is about 0.01, 0.02, 0.03, 0.04, 0.05, 0.06, 0.07, 0.08, 0.09, 0.1, 0.12, 0.14, 0.16, 0.18, 0.2, 0.25, 0.3, 0.4, 0.5, 0.6, 0.7, 0.8, 0.9, 0.99. In some embodiments, the reaction mixture includes an amount of organic solvent ranging from 70% to 130%, 80% to 120%, 90% to 110%, or 95% to 105% of the volumetric amount of the organic solvent at the cloud point of the reaction mixture.

In some embodiments where the reaction mixture is aqueous and includes one or more salts, the reaction step may be kosmotropic salt assisted. In a kosmotropic salt assisted method, the reaction mixture includes water and at least one kosmotropic salt at a concentration such that the reaction mixture is at or near its cloud point. Examples of kosmotropic salts include sodium phosphate, sodium sulfate, and ammonium sulfate. Without wishing to be bound by theory, it is thought that including a kosmotropic salt in the reaction mixture such that the reaction mixture is at or near the cloud point increases the conjugation reaction efficiency. The salt molecules can disrupt the solvation shell of separation group precursors thereby increasing interactions between the separation group precursor and the support substrate.

In some embodiments, the separation media includes a first plurality of separation ligands immobilized on the support substrate and a second plurality of separation ligands immobilized on the support substrate. In some such embodiments, method 50b may be used to prepare the separation media. FIG. 5B is a flow diagram outlining method 50b for making a separation media that includes multiple pluralities of separation ligands immobilized to the support substrate. The first plurality of separation ligands synthesized according to method 50b are made from two components a first support substrate reactive precursor and a first separation group precursor. The second plurality of separation ligands synthesized according to method 50b are made from two components a second support substrate reactive precursor and a second separation group precursor. The first support substrate precursor includes the first support substrate reactive handle. The second support substrate precursor includes a second support substrate reactive handle. The first support substrate reactive handle and the second support substrate reactive handle may be the same or different. The first separation group precursor includes a separation group and a first separation group reactive handle. The second separation group precursor includes a separation group and the second separation group reactive handle. The first support substrate reactive handle and the first separation group reactive handle are cooperative reactive handles. The second support substrate reactive handle and second separation group reactive handle are cooperative handles.

Method 50b includes reacting the first support substrate precursor and the first separation group precursor such that a first reaction product is formed between the first support substrate reactive handle (of the first support substrate precursor) and the first separation group reactive handle (of the first separation group precursor). The method further includes reacting the second support substrate precursor with the second separation group precursor such that a second reaction product is formed between the second support substrate reactive handle (of the second support substrate precursor) and the second separation group reactive handle (of the second separation group precursor).

In some embodiments, the first separation group precursor includes an amine, and the entire method (50b) is amine assisted.

In some embodiments, step 54, step 56, or both are organic solvent assisted or kosmotropic salt assisted. For example, step 54 may be accomplished with a first reaction mixture that includes the first separation group precursor, water, and an organic solvent that is miscible with water or a kosmotropic salt. Step 56 may be accomplished with a second reaction mixture. The second reaction mixture includes the second separation group precursor water and an organic solvent that is miscible with water or at least one kosmotropic salt.

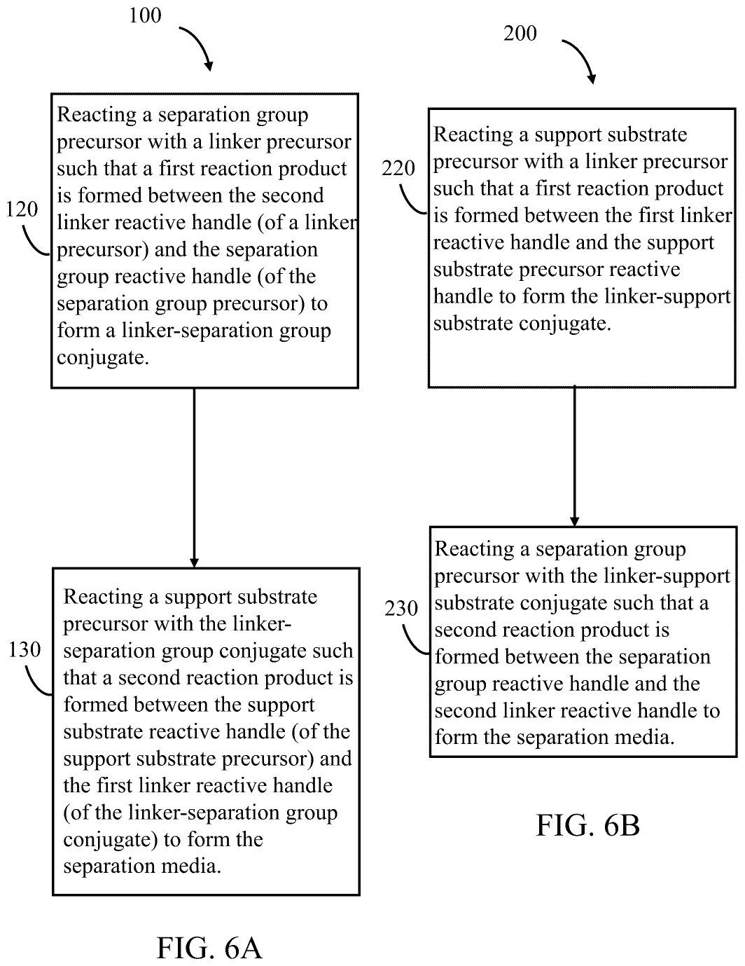

In some embodiments, method 10a, 10b, 50a, or 50b may include method 100. FIG. 6A is a flow diagram outlining method 100. Method 100 may be understood in reference to synthetic scheme S2 as described herein; however, it is understood that the method of 100 is not limited to the synthetic scheme S2. The separation ligands of the separation media made according to method 100 are synthesized from three components, a linker precursor (e.g., Pre-L), a support substrate precursor (e.g., Pre-M(2)), and a separation group precursor (e.g., Pre-Z(2)). The linker precursor includes a first linker reactive handle (Rh³), a second linker reactive handle (Rh⁴), and a spacer (Sp) that covalently links the first linker reactive handle and the second linker reactive handle. The separation group precursor includes a separation group (Z) and a separation group reactive handle (Rh⁶). The support substrate precursor includes a support substrate (thick vertical black line) and a support substrate reactive handle (Rh⁵). The second linker reactive handle (Rh⁴) and the separation group reactive handle (Rh⁶) are cooperative reactive handles. The first linker reactive handle (Rh³) and the support substrate reactive handle (Rh⁵) are cooperative reactive handles.

The method 100 includes reacting the separation group precursor with the linker precursor such that a first reaction product is formed between the second linker reactive handle (of a linker precursor) and the separation group reactive handle (of the separation group precursor) to form a linker-separation group conjugate (step 120). Method 100 further includes reacting the support substrate precursor with the linker-separation group conjugate of step 120 such that a second reaction product is formed between the support substrate reactive handle (of the support substrate precursor) and the first linker reactive handle (of the linker-separation group conjugate) to form the separation media (step 130).

In some embodiments, method 10a, 10b, 50a, or 50b may include the method 200. FIG. 6B is a flow diagram outline method 200. The method 200 may be understood in reference to synthetic scheme S3 as described herein; however, it is understood that the method of 200 is not limited to the synthetic scheme S3. The separation ligand made according to method 200 is synthesized from three components, a linker precursor (e.g., Pre-L), a support substrate precursor (e.g., Pre-M(2)), and a separation group precursor (e.g., Pre-Z(2)). The linker precursor includes a first linker reactive handle (Rh³), a second linker reactive handle (Rh⁴), and a spacer (Sp) that covalently links the first linker reactive handle and the second linker reactive handle. The separation group precursor includes a separation group (Z) and a separation group reactive handle (Rh⁶). The support substrate precursor includes a support substrate (M) and a support substrate reactive handle (Rh⁵). The second linker reactive handle (Rh⁴) and the separation group reactive handle (Rh⁶) are cooperative reactive handles. The first linker reactive handle (Rh³) and the support substrate reactive handle (Rh⁵) are cooperative reactive handles.

Method 200 includes reacting a support substrate precursor with a linker precursor such that a first reaction product is formed between the first linker reactive handle and the support substrate precursor reactive handle to form the linker-support substrate conjugate. Method 200 further includes reacting a separation group precursor with the linker-support substrate conjugate such that a second reaction product is formed between the separation group reactive handle and the second linker reactive handle to form the separation media.

Any step of method 100 or method 200 may be organic solvent assisted or kosmotropic salt assisted. In some embodiments, methods 10a, 10b, 50a, 50b, 100, and 200 further include functionalizing the support substrate to install the support substrate reactive handles. Installing the support substrate reactive handles followed by one or more conjugation reactions to immobilize the separation ligands to the reactive handles is called indirect immobilization. In such embodiments, the method may further include depositing a polymer having reactive handles onto the support substrate. In some embodiments, the polymer is deposited such that is grafted onto the support substrate. In other embodiments, the polymer is deposited such that it is grafted from the support substrate. In embodiments where the polymer is grafted from the support substrate, the method may further include coupling an initiator to the support substrate to form an immobilized initiator. In such embodiments, the method may further include polymerizing a plurality of monomers from the immobilized initiator.

In some embodiments, the support substrate reactive handle is already a part of the support substrate and not from a deposited functional layer. In such embodiments, the separation ligands are immobilized directly to the support substrate in a process called direct immobilization. Any of the methods 10a, 10b, 50a, 50b, 100, and 200 may include direct immobilization.

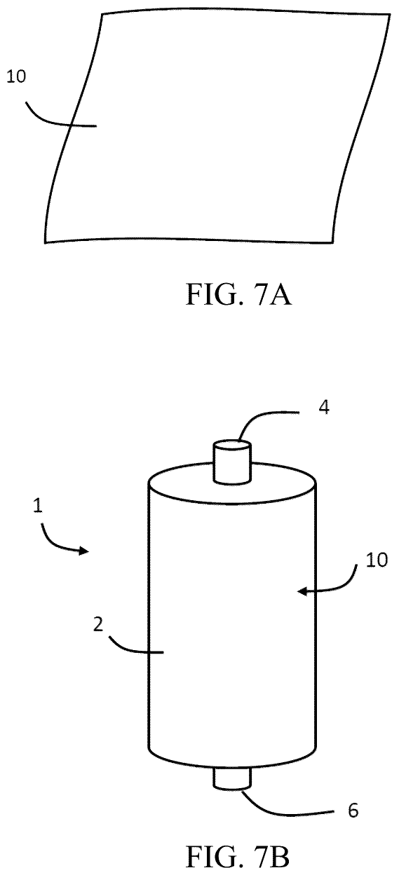

Direct and indirect immobilization may be accomplished using the amine assisted method, without amine assistance groups (not amine assisted), using the organic solvent assistance method, not using the organic solvent assistance method, using the kosmotropic assisted method, not using the kosmotropic salt assisted method, or any combination thereof. The separation media of the present disclosure may be employed in a separation device. The separation device may be a membrane chromatography column, a membrane chromatography cassette, or other membrane chromatography device that includes the separation media of the present disclosure. A separation device may be operated manually or integrated with software, pumps, detectors, and other accessories. The separation media 10 is schematically shown as a membrane in FIG. 7A. The separation media membrane 10 may be provided in a separation device 1 (e.g., a chromatography column), shown in FIG. 7B. The separation device 1 includes a housing 2 with an inlet 4 and an outlet 6 to facilitate flow through the device.

In some embodiments, two or more separation media of the present disclosure may be arranged in a stacked configuration. The stacked configuration may be employed in a separation device. In some embodiments, a first separation media and a second separation media are arranged in a stacked configuration. In some embodiments, the first separation media and the second separation media have the same identity; that is, the separation media have the same support substrate and the same separation ligands immobilized on the substrate. The separation ligands are immobilized at the same or similar separation ligand densities. In other embodiments, the first separation media and the second separation media have different identities. For example, the first separation media and the second separation media have a different support substrate; different separation ligands; different separation group densities; or any combination thereof.

The separation device (e.g., membrane chromatography column, membrane chromatography cassette, or other membrane chromatography device) may provide a residence time of 5 minutes or less, 2 minutes or less, 1 minute or less, 30 seconds or less, 10 seconds or less, 6 seconds or less, 5 seconds or less, 4 seconds or less, 3 seconds or less, 2 seconds or less, or 1 second or less. The separation device (e.g., membrane chromatography column, membrane chromatography cassette, or other membrane chromatography device) may provide a residence time of 0.01 seconds or greater, 0.1 seconds or greater, 1 second or greater, 5 seconds or greater, 6 seconds or greater, 10 seconds or greater, 30 seconds or greater, 1 minute or greater, or 2 minutes or greater. Residence time is the time any normalized amount of fluid takes to traverse the separation media of the separation device (a single separation media or multiple separation media). For example, residence time is the time it takes any molecule that is not the target and/or does not bind to the separation media to traverse the separation media in a separation device. Residence time is calculated as the flow rate or the solution going through the column divided by the total bed volume of all of the separation media included in the separation device. The residence times of the separation devices of the present disclosure may be lower than those of separation media made of resins. According to an embodiment, using membrane-based purification devices can significantly improve productivity.

Process productivity can be defined using the equation below. In the denominator, Vₜₒₜ is the total volume of solution passing through the separation media (e.g., column or cassette) during the whole process, including load (the volume of the isolation solution discussed herein), rinse (e.g., the volume of the washing solution as discussed herein), elution (e.g., the volume of the elution solution as discussed herein), and regeneration steps (e.g., the volume of the regeneration solution as discussed herein). BV is the chromatography medium bed volume (corresponding to the volume of the separation media), and τ (tau) is residence time. Loading volume is proportional to dynamic binding capacity of the chromatography column medium. Thus, process productivity increases with increasing binding capacity and decreasing residence time.

$$\\ Productivity = \frac{target}{Cost of time} = \frac{Loading volume \times target \times yield}{( \frac{{V}_{tot}}{BV} ) \times τ}$$

Dynamic binding capacity generally refers to the concentration of bound target on the separation media (milligram bound per unit bed volume of separation media) at breakthrough in the effluent. A dynamic binding capacity at 10% breakthrough (DBC_{10%}) can be determined via a standard chromatography method, e.g., using Cytiva ÄKTA pure Fast Protein Liquid chromatography (FPLC). First, the separation media is packed into a housing unit. Then, the contained separation media is connected to an FPLC system. Next, feed material (e.g., isolation solution) containing the target is passed though the separation media under certain column volumes per minute flowrate (CV/min) until the effluent concentration of the target reaches 10% of the feed concentration, as determined by a detector (e.g., a UV detector) . At the end, based on the holdup volume in the FPLC system and separation media volume, the DBC_{10%} is calculated as follows ((Volume to 10% breakthrough-holdup volume)×(feed concentration))/(volume of separation media)=DBC_{10%} expressed as mg target material/unit volume separation media. The volume of the separation media is determined by the surface area of the separation media multiplied by the thickness of the separation media. The volume of the separation media can be referred to as the bed volume. In general, the volume of the separation media does not account for the void space within the separation media. The holdup volume is the total volume between the injection port (i.e., the location where a fluid enters the system) and the detector. The holdup volume includes the bed volume (e.g., the separation media volume) as well as any volume between the injection port and the bed and any volume between the bed and the detector.

In some embodiments, a separation media or separation device containing the same has a dynamic binding capacity at 10% breakthrough of 0.01 milligrams of target per 1 mL bed volume (mg/mL bed volume) or greater, 0.1 mg/mL bed volume or greater, 1 mg/mL bed volume or greater, 5 milligrams of target per 1 mL of separation media (mg/mL bed volume) or greater, 10 mg/mL bed volume or greater, 20 mg/mL bed volume or greater, 25 mg/mL bed volume or greater, 30 mg/mL bed volume or greater, 35 mg/mL bed volume or greater, 40 mg/mL bed volume or greater, 45 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, or 120 mg/mL bed volume or greater. In some embodiments, a separation media has a dynamic binding capacity at 10% breakthrough of 150 mg/mL bed volume or less, 120 mg/mL bed volume or less100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 35 mg/mL bed volume or less, 30 mg/mL bed volume or less, 25 mg/mL bed volume or less, 20 mg/mL bed volume or less, 10 mg/mL bed volume or less, 5 mg/mL bed volume or less, 1 mg/mL bed volume or less, or 0.1 mg/mL bed volume or less. In some embodiments, a separation media or separation device containing the same has a dynamic binding capacity at 10% breakthrough of 0.01 mg/mL bed volume to 150 mg/mL bed volume, 0.01 mg/mL bed volume to 120 mg/mL bed volume, 0.01 mg/mL bed volume to 100 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 150 mg/mL bed volume, 0.1 mg/mL bed volume to 120 mg/mL bed volume, 0.1 mg/mL bed volume to 100 mg/mL bed volume, 0.1 mg/mL bed volume to 10 mg/mL bed volume, 0.1 mg/mL bed volume to 5 mg/mL bed volume, 0.1 mg/mL bed volume to 1 mg/ml bed volume, 1 mg/mL bed volume to 150 mg/mL bed volume, 1 mg/mL bed volume to 120 mg/mL bed volume, 1 mg/mL bed volume to 100 mg/mL bed volume, 5 mg/mL bed volume to 150 mg/mL bed volume, 5 mg/mL bed volume to 120 mg/mL bed volume, 5 mg/mL bed volume to 100 mg/mL bed volume, 10 mg/mL bed volume to 150 mg/mL bed volume, 10 mg/mL bed volume to 120 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 15 mg/mL bed volume to 60 mg/mL bed volume, 15 mg/mL bed volume to 50 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume. The dynamic binding capacity may depend at least in part on the target and the affinity group.

The separation media of the present disclosure may have a variety of static binding capacities (SBC). The static binding capacity is the amount of target bound to the separation media per volume of the separation media. The static binding capacity can be determined, for example, by incubating the separation media with an isolation solution containing a known amount of the target ligand for a period of time. Following incubation, the amount of the target still in the isolation solution (target not bound to the separation media) can be measured. The static binding capacity can then be calculated as the difference between the initial amount of the target in the isolation solution and the amount of target in the isolation solution following incubation with the separation media. The amount of the target in the isolation solution pre- and post-incubation with the separation media can be determined, for example, using spectroscopy and/or high performance liquid chromatography.

The static binding capacity may be higher than the dynamic binding capacity at 10% breakthrough. For example, in some embodiments, the SBC can be 10% to 40% greater than the DBC_{10%}. The pore size of the support substrate may influence the SBC and DBC_{10%}. For example, smaller pore sizes may cause a greater difference between the SBC and the DBC_{10%} as compared to relatively larger pore sizes. In some embodiments, a separation media or separation device containing the same has a static binding capacity of 0.01 milligrams of target per 1 mL of bed volume (mg/mL bed volume) or greater, 0.1 mg/mL bed volume or greater, 1 mg/mL bed volume or greater, 5 milligrams of target per 1 mL of separation media (mg/mL bed volume) or greater, 10 mg/mL bed volume or greater, 20 mg/mL bed volume or greater, 25 mg/mL bed volume or greater, 30 mg/mL bed volume or greater, 35 mg/mL bed volume or greater, 40 mg/mL bed volume or greater, 45 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, or 120 mg/mL bed volume or greater. In some embodiments, a separation media or separation device containing the same has a static binding capacity of 150 mg/mL bed volume or less, 120 mg/ml bed volume or less100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 35 mg/mL bed volume or less, 30 mg/mL bed volume or less, 25 mg/mL bed volume or less, 20 mg/mL bed volume or less, 10 mg/mL bed volume or less, 5 mg/mL bed volume or less, 1 mg/mL bed volume or less, or 0.1 mg/mL bed volume or less. In some embodiments, a separation media or separation device containing the same has a static binding capacity of 0.01 mg/mL bed volume to 150 mg/mL bed volume, 0.01 mg/mL bed volume to 120 mg/mL bed volume, 0.01 mg/mL bed volume to 100 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 150 mg/mL bed volume, 0.1 mg/mL bed volume to 120 mg/mL bed volume, 0.1 mg/mL bed volume to 100 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, 1 mg/mL bed volume to 150 mg/mL bed volume, 1 mg/mL bed volume to 120 mg/mL bed volume, 1 mg/mL bed volume to 100 mg/mL bed volume, 5 mg/mL bed volume to 150 mg/mL bed volume, 5 mg/mL bed volume to 120 mg/mL bed volume, 5 mg/mL bed volume to 100 mg/mL bed volume, 10 mg/mL bed volume to 150 mg/mL bed volume, 10 mg/mL bed volume to 120 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 15 mg/mL bed volume to 60 mg/mL bed volume, 15 mg/mL bed volume to 50 mg/mL bed volume, 20 mg/mL bed volume to 80 mg/mL bed volume, 20 mg/mL bed volume to 70 mg/ml bed volume, 20 mg/mL bed volume to 60 mg/mL bed volume, 20 mg/mL bed volume to 50 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume. The static binding capacity may depend at least in part on the target and the affinity group.

The separation media may have a variety of separation ligand densities. Separation ligand density is the amount of separation ligands immobilized per unit volume of the separation media. In embodiments where the separation media only includes separation groups that include affinity groups, the separation group density can be a measure of affinity group density. The separation ligand density can be determined, for example, by incubating the support substrate (for example, according to S1, S2, or S3) with the reaction solution containing a known amount of the separation group precursor for immobilization for a reaction time to form the separation media. Following incubation, the amount of the separation group precursor containing still in the reaction solution (unreacted) can be measured. The density of the separation ligands can then be calculated as the difference between the initial amount of the separation group precursor in the reaction solution and the amount of separation group precursor in the reaction solution following incubation with the support substrate. The amount of the separation group precursor in the reaction solution pre- and post-incubation with the support substrate can be determined, for example, using spectroscopy and/or high performance liquid chromatography. The separation group pre-cursor can be used as a proxy for the separation ligand.

In some embodiments, the separation media has separation ligand density of 0.01 milligrams of separation ligand per 1 mL of bed volume (mg/mL bed volume) or greater, 0.1 mg/mL bed volume or greater, 1 mg/mL bed volume or greater, 5 mg/mL bed volume or greater, 10 mg/mL bed volume or greater, 20 mg/mL bed volume or greater, 30 mg/mL bed volume or greater, 40 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, 110 mg/mL bed volume or greater, or 120 mg/mL bed volume or greater. In some embodiments, a separation media has a separation ligand density of 150 mg/mL bed volume or less, 120 mg/mL bed volume or less, 110 mg/mL bed volume or less, 100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 30 mg/mL bed volume or less, or 20 mg/mL bed volume or less, 10 mg/mL bed volume or less, 5 mg/mL bed volume or less, 1 mg/mL bed volume or less, or 0.1 mg/mL bed volume or less. In some embodiments, a separation media has a separation ligand density of 0.01 mg/mL bed volume to 150 mg/mL bed volume, 0.1 mg/mL bed volume to 150 mg/mL bed volume, 1 mg/mL bed volume to 150 mg/mL bed volume, 5 mg/mL bed volume to 150 mg/mL bed volume, 10 mg/ml bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 10 mg/mL bed volume to 20 mg/ml bed volume, 15 mg/mL bed volume to 60 mg/mL bed volume, 15 mg/mL bed volume to 50 mg/mL bed volume, 15 mg/mL bed volume to 30 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 20 mg/mL bed volume to 30 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, or 1 mg/mL bed volume to 10 mg/mL bed volume.

Separation ligand density can also be described as the specific surface area (SSA) in square meters (m²) relative to the bed volume of the separation media. SSA can be determined, for example, using nitrogen Brunauer-Emmett-Teller (BET) analysis. Prior to immobilization of the separation ligands on the support substrate, the support substrate will have a support substrate SSA. After immobilization of the separation ligands on the support substrate to form the separation media, the separation media has a separation media SSA. The support substrate SSA and the separation media SSA may be impacted by the pore size of the support substrate, Generally, support substrates with greater pore sizes have a larger support substrate SSA. Generally, the separation media SSA will be greater than the support substrate SSA. In some embodiments the separation media SSA is 0.5 times or greater than the support substrate SSA, 1 time or greater than the support substrate SSA, 1.5 times or greater than the support substrate SSA, 2 times or greater than the support substrate SSA, 3 time or greater than the support substrate SSA, 4 time or greater than the support substrate SSA, 5 time or greater than the support substrate SSA, or 7 time or greater than the support substrate SSA. In some embodiments the separation media SSA is 10 times or less than the support substrate SSA, 7 times or less than the support substrate SSA, 5 times or less than the support substrate SSA, 4 times or less than the support substrate SSA, 3 times or less than the support substrate SSA, 2 times or less than the support substrate SSA, 1.5 times or less than the support substrate SSA, or 1 time or less than the support substrate SSA.

In some embodiments the separation media has a separation SSA of 1.5 meters squared per milliliter of bed volume (m²/mL bed volume) or greater, 2 m²/mL bed volume or greater, 3 m²/mL bed volume or greater, 4 m²/mL bed volume or greater, 5 m²/mL bed volume or greater, 8 m²/mL bed volume or greater, 9 m²/mL bed volume or greater, 10 m²/mL bed volume or greater, or 15 m²/mL bed volume when the support substrate has an average pore size of 0.1 μm to 10.0 μm, such as 0.2 μm to 0.5 μm. In some embodiments the separation media has a separation SSA of 20 m²/mL bed volume or less, 15 m²/mL bed volume or less, 10 m²/mL bed volume or less, 9 m²/mL bed volume or less, 8 m²/mL bed volume or less, 7 m²/mL bed volume or less, 6 m²/mL bed volume or less, 5 m²/mL bed volume or less, 4 m²/mL bed volume or less, or 3 m²/mL bed volume or less, 2 m²/mL bed volume or less when the support substrate has an average pore size of 0.1 μm to 10.0 μm, such as 0.2 μm to 0.5 μm. In some embodiments the separation media has a separation SSA of 1.5 m²/mL bed volume to 20 m²/mL bed volume, 1.5 m²/mL bed volume to 15 m²/mL, 1.5 m²/mL bed volume to 10 m²/mL, 2 m²/mL bed volume to 20 m²/mL, 2 m²/mL bed volume to 15 m²/mL, 2 m²/mL bed volume to 10 m²/mL, 2 m²/mL bed volume to 9 m²/mL, 2 m²/mL bed volume to 8 m²/mL, 2 m²/mL bed volume to 7 m²/mL, 2 m²/mL bed volume to 6 m²/mL, 2 m²/mL bed volume to 5 m²/mL, 3 m²/mL bed volume to 20 m²/mL, 3 m²/mL bed volume to 15 m²/mL, 3 m²/mL bed volume to 10 m²/mL, 4 m²/mL bed volume to 20 m²/mL, 4 m²/mL bed volume to 15 m²/mL, 4 m²/mL bed volume to 10 m²/mL, 5 m²/mL bed volume to 20 m²/mL, 5 m²/mL bed volume to 15 m²/mL, or 5 m²/mL bed volume to 10 m²/mL when the support substrate has an average pore size of 0.1 μm to 10.0 μm, such as 0.2 μm to 0.5 μm.

In some embodiments, the separation media and/or separation devices containing the same are able to purify a target molecule at a fast flow rate. For example, separation media and/or separation devices containing the same may be used to purify a target at residence times of 5 minutes or less, 2 minutes or less, 1 minute or less, 30 seconds or less, 10 seconds or less, or 6 seconds or less. The residence time is somewhat dependent on the volume of the separation media and/or on the size of the device. For example, in separation media that have low volumes and/or separation devices that are small, the residence times may be as low as 1 second or less. Although there is no desired lower limit for the residence time, in practice residence times are 0.1 seconds or greater.

In some embodiments, the separation media and/or separation device containing the same may be used to purify or concentrate a target from an isolation solution with a high recovery of the target molecule. The recovery of a target molecule is amount of the target molecule recovered after passing it through the separation media divided by the amount of target molecule in the isolation solution. In some embodiments, the target molecule recovery is 50% or greater, 60% or greater, 80% or greater, 90% or greater, 95% or greater, or 99% or greater. In some embodiments, the target molecule recovery is 100% or less, 95% or less, 90% or less, 80% or less, 70% or less, or 60% or less. In some embodiments, the target molecule recovery is 80% to 100%, 90% to 100%, or 95% to 100%.

In some embodiments, the separation media and/or separation device containing the same may be used to purify or concentrate target models from an isolation solution with a high recovery of active target molecule. An active target molecule is a molecule that possesses at least some of the function as the target molecule prior to exposure to the separation media of the present disclosure. For example, an active target molecule is a target molecule that has undergone purification using the separation media of the present disclosure and retains at least some binding affinity to a binding partner. The recovery of active target molecules is the amount of active target molecule recovered after passing them through the separation media divided by the amount of target molecules in the isolation solution. In some embodiments, the active target molecule recovery is 50% or greater, 60% or greater, 80% or greater, 90% or greater, 95% or greater, or 99% or greater. In some embodiments, the active target molecule recovery is 100% or less, 95% or less, 90% or less, 80% or less, 70% or less, or 60% or less. In some embodiments, the active target molecule recovery is 80% to 100%, 90% to 100%, or 95% to 100%.

In some embodiments, the separation media and/or separation device containing the same may be used to remove impurities from an isolation solution. An impurity is any molecule that is not the target molecule, or a buffering agent, a salt, or an additive that has been added to the isolation solution. In some embodiments, the separation media is able to remove 50% or greater, 60% or greater, 70% or greater, 80% or greater, 90% or greater, or 99% or greater of the impurities initially present in the isolation solution. In some embodiments, the separation media is able to remove 100% or less, 99% or less, 90% or less, 80% or less, 70% or less, or 60% or less. In some embodiments, the separation media is able to remove 50% to 100%, 60% to 100% , 70% to 100%, 80% to 100% 90% to 100%, 90% to 99%, or 90% to 95% of the impurities initially present in the isolation solution.

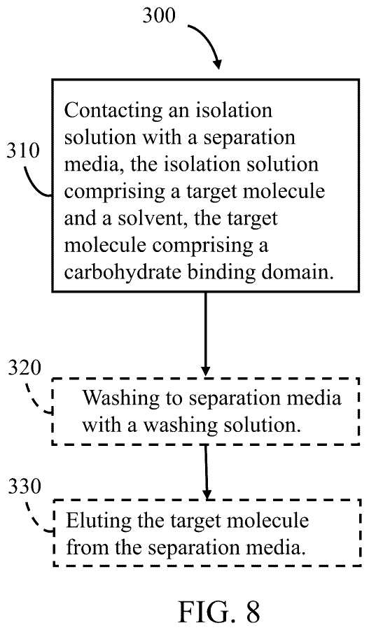

The present disclosure describes a method for using the separation media and/or the separation devices of the present disclosure. FIG. 8 is a flow diagram outlining a method 300 for using the separation media of the present disclosure to isolate and/or concentrate a target molecule from an isolation solution. Method 300 includes contacting an isolation solution with a separation media (step 310).

The isolation solution includes a solvent (isolation solvent) and a target molecule. In some embodiments, the isolation solution includes a plurality of target molecules that have already been purified from a mixture that included additional biomolecules. In such embodiments the plurality of target molecules may be already pure but not concentrated to the desired concentration in a given isolation solution. In some such embodiments, the separation media may be used to concentrate the plurality of target molecules by decreasing the volume of solution in which they are located. In such embodiments, the isolation solution may include one or more suitable buffering agents, one or more suitable salts, one or more suitable additives, or any combination thereof.

In other embodiments, the separation media or separation device may be used to purify the target from a mixture that includes contaminant molecules or undesired molecules. In some such embodiments, the isolation solution includes a mixture of biomolecules and/or cellular debris. In addition, the isolation solution may include one or more suitable buffering agents, suitable salts, other suitable additives, or any combination thereof. For example, the isolation solution may include the lysate of an expression system used to produce the plurality of target molecules as well as any salts, buffering agents, or additives used to lyse the cells. The isolation solution may include the media of an expression system in which the target molecules have been excreted from.

Examples of suitable salts and buffering agents include sodium chloride; potassium chloride; lithium chloride; rubidium chloride; calcium chloride; magnesium chloride; cesium chloride; tris base (tris(hydroxymethyl)aminomethane); 4-(2-hydroxyethyl)-1-piperazineethanesulfonic acid (HEPES) ;; sodium phosphate; potassium phosphate; ammonium sulfate, 2-(N-morpholino)ethanesulfonic acid (MES); 2,2′,2″-Nitrilotriacetic acid (ADA); N-(2-Acetamido)-2-aminoethanesulfonic acid (ACES); 3-morpholino-2-hydroxypropanesulfonic acid (MOPSO); cholamine chloride hydrochloride; 3-(N-morpholino)propanesulfonic acid (MOPS); N,N-Bis(2-hydroxyethyl)-2-aminoethanesulfonic acid (BES); 2-{[1,3-Dihydroxy-2-(hydroxymethyl)propan-2-yl]amino}ethane-1-sulfonic acid (TES); 3-(N,N-Bis [2-hydroxyethyl]amino)-2-hydroxypropanesulfonic acid (DIPSO); 3-[N-Tris(hydroxymethyl)methylamino]-2-hydroxypropanesulfonic acid (TAPSO); acetamidoglycine; piperazine-1,4 BIS(2-hydroxypropanae sulphonic acid) (POPSO); N-(Hydroxyethyl)piperazine-N′-2-hydroxypropanesulfonic acid (HEPPSO); 3-[4-(2-Hydroxyethyl)piperazin-1-yl]propane-1-sulfonic acid (HEPPS); N-(Tri(hydroxymethyl)methyl)glycine (tricine); 2-Aminoacetamide; glycylglycine; N,N-Bis(2-hydroxyethyl)glycine; N-Tris(hydroxymethyl)methyl-3-aminopropanesulfonic acid (TAPS); and the like. Suitable salts and/or buffering agents may be added in an amount of 1 millimolar (mM) or greater, 5 mM or greater, or 10 mM or greater, 20 mM or greater, 50 mM or greater, 100 mM or greater 200 mM or greater, or 500 mM or greater. Suitable salts may be added in an amount of 1 M or less, 500 mM or less, 100 mM or less, 50 mM or less, or 30 mM or less. The salts may be added in an amount ranging from 1 mM to 1 M, 1 mM to 500 mM, 1 mM to 200 mM, 1 mM to 100 mM, 1 mM to 50 mM, 5 mM to 30 mM, 5 mM to 20 mM, or 20 mM to 100 mM.

In some embodiments, the isolation solution includes one or more kosmotropic salts, one or more chaotropic salts, or both. Kosmotropic salts are known as salts that decrease the solubility of nonpolar substances in aqueous solutions. In contrast, chaotropic salts increase the solubility of nonpolar substances in aqueous solutions. In some embodiments, the amount and/or identity of a kosmotropic and/or chaotropic salts may be designed to increase the binding affinity and/or binding specificity between the target molecule and the affinity groups and/or assistance groups (if present).

Examples of kosmotropic salts that may be present in the isolation solution include ammonium sulfate, ammonium phosphate, potassium phosphate, sodium sulfate, sodium chloride, and any combination thereof. Suitable kosmotropic salts may be present in the isolation solution in an amount of 0.1 M or greater, 0.5 M or greater, or 1.0 M or greater, or 2.0 M or greater. Suitable kosmotropic salts may be present in the isolation solution in an amount of 6.0 M or less, 5.0 M or less, or 4.0 M or less. The kosmotropic salts may be added in an amount ranging from 0.1 M to 6M, 0.5 M to 2.5 M, or 0.5 M to 3.0 M.

Examples of chaotropic salts that may be present in the solution include sodium chloride, calcium chloride, magnesium chloride and any combination thereof. In some embodiments, the isolation solution includes 1 M or less, 0.5 M or less, or 0.1 M or less of chaotropic salts. In some embodiments, the isolation solution is free or substantially free of chaotropic salts.

Suitable additives include glycerol and other polyols; protease inhibitors; phosphatase inhibitors; cryoprotectants; detergents; chelating agents; reducing agents; and any combination thereof. Suitable additives may be present in the isolation solution in amounts of 0.01 mM or greater, 0.1 mM or greater, 1 mM or greater, 5 mM or greater, 10 mM or greater, or 20 mM or greater. Suitable salts may be added in an amount of 100 mM or less, 50 mM or less, 30 mM or less, 10 mM or less, 5 mM or less, or 1 mM or less. Suitable additives may be present in the isolation solution in amounts ranging from 0.01 mM to 100 mM, 1 mM to 50 mM, 5 mM to 30 mM, 5 mM to 20 mM, 0.01 mM to 5 mM, or 1 mM to 5 mM.

The isolation solution solvent may be any solvent that does not degrade or react with the target molecule. In some embodiments, the solvent is water. In some embodiments, the solvent is an organic solvent such as, for example, methanol, ethanol, isopropanol, and acetonitrile, DMSO, DMF, or combinations thereof. In some embodiments, the majority of the solvent is water. Alternatively, in some embodiments, the majority of the solvent may be made up of organic solvents. In some embodiments, the solvent is nonaqueous, e.g., consists of organic solvents.

The pH of the isolation solution may be any pH that does not make the target molecule unstable or insoluble. Additionally, the pH of the isolation solution should be such that the separation ligands of the separation media are not unstable. The pH of the isolation solution may be controlled to enhance the binding affinity of the target molecules to the affinity groups and/or assistance group (if present).

The isolation solution solvent may be any solvent that does not degrade or react with the target molecule. In some embodiments, the solvent includes water, an organic solvent, or both. In some embodiments, the includes is an organic solvent such as, for example, methanol, ethanol, isopropanol, and acetonitrile, DMSO, DMF, or any combination thereof. In some embodiments, the majority of the solvent is water. Alternatively, in some embodiments, the majority of the solvent may be made up of organic solvents. In some embodiments, the solvent is nonaqueous, e.g., consists of organic solvents.

In some embodiments when the target molecule was recombinantly expressed, the isolation solution includes at least a portion of a cell lysate. As such, the isolation solution may include cell debris and other biomolecules that are not the target. In some embodiments, the isolation solution includes one or more salts, one or more buffering agents, and/or one or more additives employed in the processes of lysing the cells used to express the target.

In some embodiments, the method 300 includes washing the separation media with a washing solution (step 320). Washing the separation media with a washing solution includes contacting the separation media with the washing solution. Washing the separation media may allow for any molecules that are not the target molecule to be removed from the separation media. In the washing step, at least a portion of the target molecules remain bound to the affinity groups and temporarily immobilized on the support substrate.

The washing solution may include a variety of components or may simply be a solvent (e.g., water). The composition and/or pH of the washing solution should be such that none of the components degrade or react with the target molecule. Additionally, the composition and/or pH should be such that the washing solution does not decrease the affinity of the target molecule to the affinity group to a point where the target molecule is able to be removed from the affinity group and washed through the separation media. The washing solution includes a washing solvent. The washing solvent may be water, an organic solvent, or both. The washing solvent may be any solvent as described herein such as those described relative to the isolation solution. In embodiments, the washing solution includes one or more buffering agents, one or more salts, one or more additives, or any combination thereof. The one or more salts, one or more buffering agents, or one or more additives may be present in the washing solution in any amount as described relative to the isolation solution.

The pH of the washing solution may be any pH that does not make the target molecule unstable or insoluble. Additionally, the pH of the washing solution should be such that the separation ligands of the separation media are not unstable. The pH of the washing solution may be controlled to enhance the binding affinity of the target molecules to the affinity groups and/or decrease the binding affinity of any off target molecules to the affinity groups.

In some embodiments, step 320 may be repeated with additional washing solutions. The additional washing solutions may have the same composition and/or pH as the first washing solution or a different composition and/or pH than the first washing composition.

In some embodiments, method 300 further includes eluting the plurality of target molecules that were temporarily immobilized on the support substrate (step 330). The target molecules may be eluted using by contacting the separation media with an elution solution. The elution solution includes an elution solvent. The elution solvent may be any solvent as described herein (e.g., the solvent included in the washing solution and/or the isolation solution). The elution solution may be of any composition and/or pH that allows for the target molecules to be separated from the affinity groups and exit the separation media. As such, the elution solution generally is of a pH or includes a composition that decreases the affinity of the target molecule for the affinity group or has a higher affinity for the affinity group such as to compete of the target molecule. Additionally, if assistance groups are present, the elution solution generally is of a pH or includes a composition that decrease the electrostatic and/or hydrophobic interactions between the assistance groups and the target molecule.

Methods for eluting the target molecules include using an elution solution that has a higher conductivity and/or salt composition than the washing and/or isolation solution; using an elution solution that has a different pH than the washing and/or isolation solution; using an elution solution that has a different solvent or mixture of solvents than the washing and/or isolation solution; and combinations thereof. The composition of the elution solution may be designed for specific target molecules. Different target molecules (or target molecules and other molecules) may be eluted using a linear gradient elution or using a step isocratic elution.

In some embodiments, the elution solution includes high amounts of one or more salts in order to decrease the binding affinity between the target molecule and the affinity groups and/or assistance groups (if present). The salt or mixture of salts may be any salt as described herein, for example, in reference to the isolation solution. The salt or mixture of salts may be present in the elution solution in an amount of 50 mM or greater, 100 mM or greater, 150 mM or greater, 200 mM or greater, 300 mM or greater, 500 mM or greater, or 1 M or greater. The salt or mixture of salts may be present in the elution solution in an amount of 5 M or less, 1 M or less, 500 mM or less, 300 mM or less, 200 mM or less, or 100 mM or less.

In some embodiments, the amount and/or identity of a kosmotrope and/or chaotropic salts may be designed to decrease the binding affinity between the target molecules and the affinity groups and/or assistance groups (if present).

In other embodiments, the pH of the elution solution may be such as to decrease the binding affinity between the target molecules and the affinity groups. Without wishing to be bound by theory, the pH of the solution may impact the strength and/or availability of various affinity group - target molecule interactions such as hydrogen bonding interactions, electrostatic interactions, hydrophobic interactions, or combinations thereof. In some embodiments, the pH of the elution solution may be higher than the pH of the washing and/or isolation solution. In some embodiments, the pH of the elution solution may be lower than the pH of the washing/isolation solution.

In some embodiments, the elution solution may include a molecule that is bound by the target molecule and/or can compete for binding to the target molecule. Such a molecule may be present in an amount such as to compete off the target molecules from the affinity groups. To that end, in some embodiments, the elution solution includes an affinity group competitive molecule and a solvent. Different target molecules (or target molecules and other molecules) may be eluted using a linear gradient elution or using a step isocratic elution.

An affinity group competitive molecule is a molecule that binds to the target molecule, and when present at a sufficient concentration can compete off the target molecule from the affinity group. In some embodiments, the affinity group competitive molecule has a higher affinity for the target molecule than the affinity group. In other embodiments, the affinity group competitive molecule has a lower affinity for the target molecule than the affinity group. In yet other embodiments, the affinity group competitive molecule may have the same affinity for the affinity group as the target molecule.

An affinity group competitive molecule may be any molecule that binds to a given target molecule. In some embodiments, an affinity group competitive molecule is a monosaccharide, a disaccharide, or a glycan. In some such embodiments, the affinity group competitive molecule may include the same monosaccharide or glycosylation pattern as the affinity group (e.g., carbohydrate or carbohydrate containing protein) immobilized on the support substrate. In some embodiments, the affinity group competitive molecule does not include the same monosaccharide or glycosylation pattern as the affinity group (e.g., carbohydrate or carbohydrate containing protein) immobilized on the support substrate. The affinity group competitive molecule may be chosen based on the identity of the affinity group ligand. Examples of affinity group competitive molecules include mannosides (e.g., methyl mannoside), sorbitol, and N-acetyl-D-glucosamine.

An affinity group competitive molecule may be present in an elution solution at a concentration sufficient to compete off the target molecules from the affinity groups. In some embodiments, the affinity group competitive molecule may be present in an elution solution the amount of 20 mM or greater, 50 mM or greater, 100 mM or greater, 200 mM or greater, 300 mM or greater, 400 mM or greater, or 500 mM or greater. In some embodiments, the affinity group competitive molecule may be present in an elution solution the amount of 1 M or less, 500 mM or less, 400 mM or less, 300 mM or less, 200 mM or less, 100 mM or less, or 50 mM or less. In some embodiments, the affinity group competitive molecule may be present in an elution solution the amount of 20 mM to 400 mM, 50 mM to 200 mM, or 100 mM to 500 mM.

The volume of the elution solution used to elute the target molecules may vary. For example, in embodiments where the separation media is being employed to concentrate the target molecules, the volume of elution solution is less that the volume of isolation solution.

In some embodiments, the method includes regenerating the separation media. Regeneration is done to prepare the separation media (or the separation media of a separation device) for subsequent uses. Regeneration may include washing the separation media with a solution designed to strip any molecule that is not covalently attached to the support substrate form the separation media. Regeneration may also include flowing an equilibration solution through the separation media such as to prepare the separation media for future use. In some embodiments, the equilibrium solution may be the same as the isolation solution but without the target molecule or the same as the washing solution.

### Exemplary Embodiments

The following is a non-limiting list of exemplary embodiments according to the present disclosure.
Embodiment 1 is separation media that includes a support substrate and a plenarily of separation ligands of the formula SL immobilized on the support substrate. The formula SL is

-L-Z (SL),
where L is a linker and Z is a separation group. The separation group includes an affinity group. The affinity group includes a carbohydrate binding domain, a carbohydrate binding ligand, or both. The separation media may be configured for isolating a target molecule, the target molecule including a carbohydrate binding domain.
Embodiment 2. Embodiment 2 is the separation media of embodiment 1, wherein SL is or formula SL1 or SL2.

[symbol]

Rp¹-Z (SL1)

[symbol]

Rp³-Sp-Rp⁴-Z (SL2)

In formula SL1 and SL2 Rp¹, Rp³, and Rp⁴ each independently include the reaction product of any one of Rp^{A}, Rp^{B}, Rp^{C}, Rp^{D}, Rp^{E}, Rp^{F}, Rp^{G}, Rp^{H}, Rp^{I}, Rp^{J}, Rp^{K}, or an isomer thereof, wherein Rp^{A}, Rp^{B}, Rp^{C}, Rp^{D}, Rp^{E}, Rp^{F}, Rp^{G}, Rp^{H}, Rp^{I}, Rp^{J}, Rp^{K} are represented by:

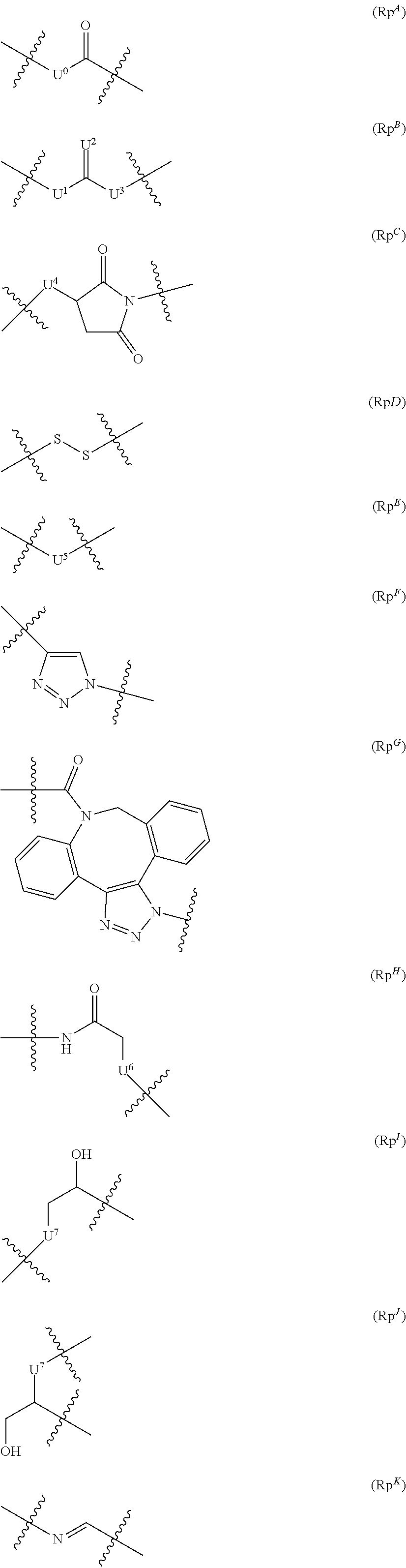

wherein U⁰, U¹, U², U³, U⁴, U⁵, U⁶, and U⁷ are each independently NH, N, O, or S. Sp is a spacer.
Embodiment 3 is the separation media of embodiment 1 or 2, where U¹, U¹, U², U³, U⁴, U⁵, U⁶, and U⁷ are each independently NH, O, or S.
Embodiment 4 is the separation media of any one of embodiments 1 to 3, wherein the plurality of separation ligands are of formula SL2 and Sp is an alkanediyl or alkenediyl comprising one or more catenated functional groups. In some embodiments, the alkanediyl or alkenediyl includes a backbone chain of length C1 to C18.
Embodiment 5 is the separation media of embodiment 4, where the alkanediyl or alkenediyl includes a backbone chain of length C1 to C3.
Embodiment 6 is the separation media of any one of embodiment 1 to 5, where the spacer includes —C(O)—.
Embodiment 7 is the separation media of any one of embodiment 1 to 6, where wherein Rp³, Rp⁴. or both includes Rp^{E}.
Embodiment 8 is the separation media of any one of embodiment 1 to 6, wherein Rp³ and Rp⁴ includes Rp^{E}.
Embodiment 9 is the separation media of embodiment 8, where each U⁵ is O.
Embodiment 10 is the separation media of embodiment 8, where each U⁵ is NH.
Embodiment 11 is the separation media of embodiment 8, where one U⁵ is NH and U⁵ is O. In some embodiments, the U⁵ of Rp³ is O and the U⁵ or Rp⁴ is NH. In some embodiments, the U⁵ of Rp³ is NH and the U⁵ or Rp⁴ is O.
Embodiment 12 is the separation media of embodiment 1 or embodiment 2, where SL2 includes

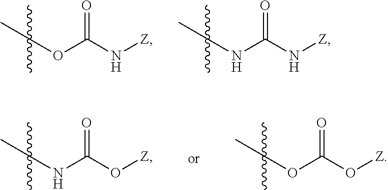

Embodiment 13 is the separation media of embodiment 1 or embodiment 2, where SL2 includes

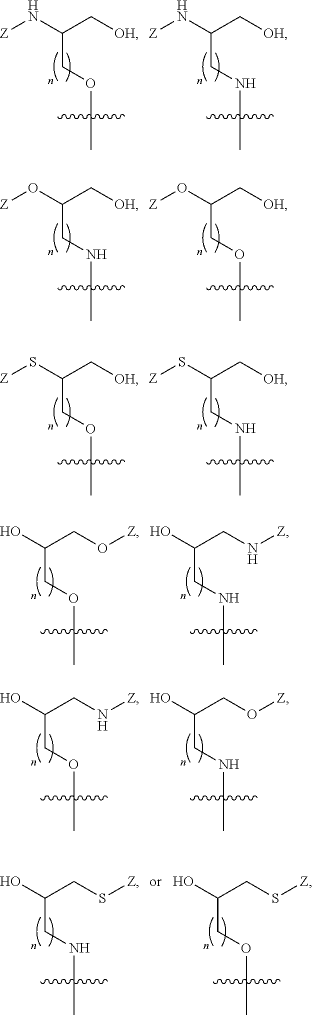

wherein n is 0, 1, 2, 3, 4, 5, 6, 7, 8, 9, or 10. In some embodiments, n is 1.

Embodiment 13 is the separation media of embodiment 1 or embodiment 2, the separation ligand formula SL or SL1 is of formula X, XI, XII, or XIII:

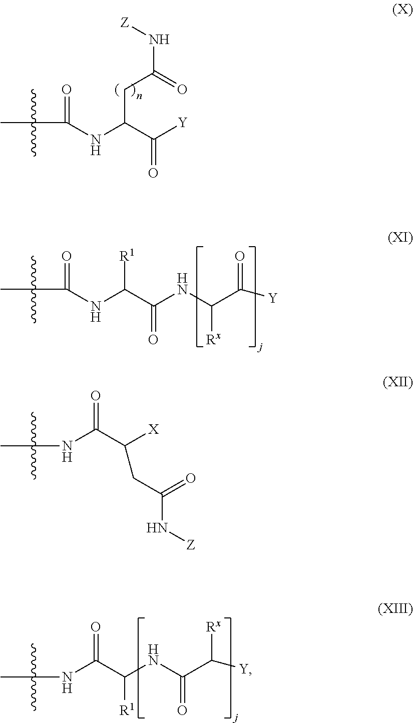

wherein n is 0, 1, 2, 3, or 4. X is NH₂ or PG_{N} where PG_{N} is an amine protecting group. Y is OH or a PG_{C(O)OH} where PG_{C(O)OH} is a carboxylic acid protecting group. R¹ is an amino acid side chain. j is 1, 2, 3, 4, 5, 6, 7, 8, 9, or 10. Each R^{X} is independently an amino acid side chain or

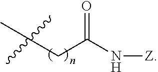

At least one R^{X} is

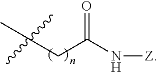

Embodiment 14 is the separation media of any one of embodiments 1 to 13, where the support substrate includes a polyolefin membrane, a polyethersulfone membrane, a poly(tetrafluoroethylene) membrane, a nylon membrane, a fiberglass membrane, a hydrogel membrane, a hydrogel monolith, a polyvinyl alcohol membrane, a cellulose membrane, a cellulose ester membrane, a cellulose acetate membrane, a regenerated cellulose membrane, a cellulosic nanofiber membrane, a cellulosic monolith, a filter paper, or any combination thereof.
Embodiment 15 is the separation media of any one of embodiments 1 to 14, where the separation media is configured for use with an organic solvent.
Embodiment 16 is the separation media of any one of embodiments 1 to 15, where the separation media is configured for use with an aqueous solvent.
Embodiment 17 is the separation media of any one of embodiments 1 to 16, where the carbohydrate includes a glycan.
Embodiment 18 is the separation media embodiment 17, where the glycan has a linear structure.
Embodiment 19 is the separation media embodiment 17, where the glycan has a branched structure with two terminal ends.
Embodiment 20 is the separation media of any one of embodiments 17 to 20, where the glycan includes a terminal sialic acid and an adjacent saccharide, the terminal sialic acid and the adjacent saccharide covalently linked through a linkage.
Embodiment 21 is the separation media of embodiment 20, where the linkage is an α-2,6 linkage.
Embodiment 22 is the separation media of embodiment 20, where the linkage is an α-2,3 linkage.
Embodiment 23 is the separation media of any one of embodiments 20 to 22, where the sialic acid is N-acetylneuraminic acid.
Embodiment 24 is the separation media of any one of embodiments 1 to 23, where carbohydrate binding domain includes hemagglutinin. In some embodiments, the hemagglutinin comprises H1, H2, H3, H4, H5, H6, H7, H8, H9, H10, H11, H12, H13, H14, H15, H16, H17, H18, or a recombinant form thereof, of influenza A.

Embodiment 25 is the separation media of any one of embodiments 1 to 24, wherein the target molecule includes a recombinant hemagglutinin or a fragment thereof. In some embodiments, the target molecule includes a recombinant hemagglutinin vaccine. In some embodiments, the target molecule includes a virus, a modified virus, or a viral vector where the virus, modified virus, or viral vector that includes hemagglutinin or a fragment thereof (for example, influenza A, influenza B, influenza C, or influenza D). In some embodiments, the target includes the HA1 subunit (eHA1) of the influenza A virus subtype HIN1.
Embodiment 26 is a separation media comprising two or more of the separation media of any one of embodiment 1 to 25 arranged in a stacked configuration. In some embodiments, the separation media includes two separation media and the separation media are of the same identity. In some embodiments, the separation media includes two separation media and the separation media are of a different identity.
Embodiment 27 is a separation device comprising a housing and the separation media of any one of embodiments 1 to 26 disposed within the housing.
Embodiment 28 is a method of isolating a target molecule from an isolation solution. The isolation solution includes an isolation solvent and the target molecule comprising a carbohydrate. The target molecule may be any target molecule of embodiment 24 to 25. The method includes contacting the isolation solution with the separation media of any one of embodiment 1 to 26 or the separation media within the separation device of embodiment 27.
Embodiment 29 is a method of embodiment 28, where the method further includes washing the separation media with a washing solution.
Embodiment 30 is a method of embodiment 28 or 29, where the method further includes eluting the target molecule from the separation media. Eluting may use an elution solution comprising an elution solvent and an affinity group competitive molecule.
Embodiment 31 is the separation media, separation device, or method of any one of embodiments 1 to 30, wherein the separation media has a static binding capacity of has a static binding capacity of 0.01 milligrams of target per 1 mL of bed volume (mg/mL bed volume) or greater, 0.1 mg/mL bed volume or greater, 1 mg/mL bed volume or greater, 5 milligrams of target per 1 mL of separation media (mg/mL bed volume) or greater, 10 mg/mL bed volume or greater, 20 mg/mL bed volume or greater, 25 mg/mL bed volume or greater, 30 mg/mL bed volume or greater, 35 mg/mL bed volume or greater, 40 mg/mL bed volume or greater, 45 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, or 120 mg/mL bed volume or greater. In some embodiments, a separation media or separation device containing the same has a static binding capacity of 150 mg/mL bed volume or less, 120 mg/mL bed volume or less 100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 35 mg/mL bed volume or less, 30 mg/mL bed volume or less, 25 mg/mL bed volume or less, 20 mg/mL bed volume or less, 10 mg/mL bed volume or less, 5 mg/mL bed volume or less, 1 mg/mL bed volume or less, or 0.1 mg/mL bed volume or less. In some embodiments, a separation media or separation device containing the same has a static binding capacity of 0.01 mg/mL bed volume to 150 mg/mL bed volume, 0.01 mg/mL bed volume to 120 mg/mL bed volume, 0.01 mg/mL bed volume to 100 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 150 mg/mL bed volume, 0.1 mg/mL bed volume to 120 mg/mL bed volume, 0.1 mg/mL bed volume to 100 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, 1 mg/mL bed volume to 150 mg/mL bed volume, 1 mg/mL bed volume to 120 mg/mL bed volume, 1 mg/mL bed volume to 100 mg/mL bed volume, 5 mg/mL bed volume to 150 mg/mL bed volume, 5 mg/mL bed volume to 120 mg/mL bed volume, 5 mg/mL bed volume to 100 mg/mL bed volume, 10 mg/mL bed volume to 150 mg/mL bed volume, 10 mg/mL bed volume to 120 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 15 mg/mL bed volume to 60 mg/mL bed volume, 15 mg/mL bed volume to 50 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume.
Embodiment 32 is the separation media, separation device, or method of any one of embodiments 1 to 31, wherein the separation media has a dynamic binding capacity at 10% breakthrough of 0.01 milligrams of target per 1 mL bed volume (mg/mL bed volume) or greater, 0.1 mg/mL bed volume or greater, 1 mg/mL bed volume or greater, 5 milligrams of target per 1 mL of separation media (mg/mL bed volume) or greater, 10 mg/mL bed volume or greater, 20 mg/mL bed volume or greater, 25 mg/mL bed volume or greater, 30 mg/mL bed volume or greater, 35 mg/mL bed volume or greater, 40 mg/mL bed volume or greater, 45 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, or 120 mg/mL bed volume or greater. In some embodiments, a separation media has a dynamic binding capacity at 10% breakthrough of 150 mg/mL bed volume or less, 120 mg/mL bed volume or less100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 35 mg/mL bed volume or less, 30 mg/mL bed volume or less, 25 mg/mL bed volume or less, 20 mg/mL bed volume or less, 10 mg/mL bed volume or less, 5 mg/mL bed volume or less, 1 mg/mL bed volume or less, or 0.1 mg/mL bed volume or less. In some embodiments, a separation media or separation device containing the same has a dynamic binding capacity at 10% breakthrough of 0.01 mg/mL bed volume to 150 mg/mL bed volume, 0.01 mg/mL bed volume to 120 mg/mL bed volume, 0.01 mg/mL bed volume to 100 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 150 mg/mL bed volume, 0.1 mg/mL bed volume to 120 mg/mL bed volume, 0.1 mg/mL bed volume to 100 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, 1 mg/mL bed volume to 150 mg/mL bed volume, 1 mg/mL bed volume to 120 mg/mL bed volume, 1 mg/mL bed volume to 100 mg/mL bed volume, 5 mg/mL bed volume to 150 mg/mL bed volume, 5 mg/mL bed volume to 120 mg/mL bed volume, 5 mg/mL bed volume to 100 mg/mL bed volume, 10 mg/mL bed volume to 150 mg/mL bed volume, 10 mg/mL bed volume to 120 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 15 mg/mL bed volume to 60 mg/mL bed volume, 15 mg/mL bed volume to 50 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume.
Embodiment 33 is the separation media, separation device, or method of any one of embodiments 1 to 32, wherein the separation media has a separation ligand density of 0.01 milligrams of separation ligands per 1 mL of bed volume (mg/mL bed volume) or greater, 0.1 mg/mL bed volume or greater, 1 mg/mL bed volume or greater, 5 mg/mL bed volume or greater, 10 mg/mL bed volume or greater, 20 mg/mL bed volume or greater, 30 mg/mL bed volume or greater, 40 mg/mL bed volume or greater, 50 mg/mL bed volume or greater, 60 mg/mL bed volume or greater, 70 mg/mL bed volume or greater, 80 mg/mL bed volume or greater, 90 mg/mL bed volume or greater, 100 mg/mL bed volume or greater, 110 mg/mL bed volume or greater, or 120 mg/mL bed volume or greater. In some embodiments, a separation media has a separation ligand density of 150 mg/mL bed volume or less, 120 mg/mL bed volume or less, 110 mg/mL bed volume or less, 100 mg/mL bed volume or less, 90 mg/mL bed volume or less, 80 mg/mL bed volume or less, 70 mg/mL bed volume or less, 60 mg/mL bed volume or less, 50 mg/mL bed volume or less, 40 mg/mL bed volume or less, 30 mg/mL bed volume or less, or 20 mg/mL bed volume or less, 10 mg/mL bed volume or less, 5 mg/mL bed volume or less, 1 mg/mL bed volume or less, or 0.1 mg/mL bed volume or less. In some embodiments, a separation media has a separation ligand density of 0.01 mg/mL bed volume to 150 mg/mL bed volume, 0.1 mg/mL bed volume to 150 mg/mL bed volume, 1 mg/mL bed volume to 150 mg/mL bed volume, 5 mg/mL bed volume to 150 mg/mL bed volume, 10 mg/mL bed volume to 100 mg/mL bed volume, 10 mg/mL bed volume to 90 mg/mL bed volume, 10 mg/mL bed volume to 80 mg/mL bed volume, 10 mg/mL bed volume to 70 mg/mL bed volume, 10 mg/mL bed volume to 60 mg/mL bed volume, 10 mg/mL bed volume to 50 mg/mL bed volume, 10 mg/mL bed volume to 40 mg/mL bed volume, 10 mg/mL bed volume to 20 mg/mL bed volume, 15 mg/mL bed volume to 60 mg/mL bed volume, 15 mg/mL bed volume to 50 mg/mL bed volume, 15 mg/mL bed volume to 30 mg/mL bed volume, 20 mg/mL bed volume to 80 mg/mL bed volume, 20 mg/mL bed volume to 70 mg/mL bed volume, 20 mg/mL bed volume to 60 mg/mL bed volume, 20 mg/mL bed volume to 50 mg/mL bed volume, 20 mg/mL bed volume to 30 mg/mL bed volume, 30 mg/mL bed volume to 80 mg/mL bed volume, 30 mg/mL bed volume to 70 mg/mL bed volume, 30 mg/mL bed volume to 60 mg/mL bed volume, or 30 mg/mL bed volume to 50 mg/mL bed volume, 0.01 mg/mL bed volume to 10 mg/mL bed volume, 0.01 mg/mL bed volume to 5 mg/mL bed volume, 0.01 mg/mL bed volume to 1 mg/mL bed volume, 0.1 mg/mL bed volume to 10 mg/mL bed volume, 0.1 mg/mL bed volume to 5 mg/mL bed volume, 0.1 mg/mL bed volume to 1 mg/mL bed volume, or 1 mg/mL bed volume to 10 mg/mL bed volume.
Embodiment 34 is the separation media, separation device, or method of any one of embodiments 1 to 33, wherein the separation media has a separation specific surface area (SSA) of 1.5 meters squared per milliliter of bed volume (m²/mL bed volume) or greater, 2 m²/mL bed volume or greater, 3 m²/mL bed volume or greater, 4 m²/mL bed volume or greater, 5 m²/mL bed volume or greater, 8 m²/mL bed volume or greater, 9 m²/mL bed volume or greater, 10 m²/mL bed volume or greater, or 15 m²/mL bed volume when the support substrate has an average pore size of 0.1 μm to 10.0 μm, such as 0.2 μm to 0.5 μm. In some embodiments the separation media has a separation SSA of 20 m²/mL bed volume or less, 15 m²/mL bed volume or less, 10 m²/mL bed volume or less, 9 m²/mL bed volume or less, 8 m²/mL bed volume or less, 7 m²/mL bed volume or less, 6 m²/mL bed volume or less, 5 m²/mL bed volume or less, 4 m²/mL bed volume or less, or 3 m²/mL bed volume or less, 2 m²/mL bed volume or less when the support substrate has an average pore size of 0.1 μm to 10.0 μm, such as 0.2 μm to 0.5 μm. In some embodiments the separation media has a separation SSA of 1.5 m²/mL bed volume to 20 m²/mL bed volume, 1.5 m²/mL bed volume to 15 m²/mL, 1.5 m²/mL bed volume to 10 m²/mL, 2 m²/mL bed volume to 20 m²/mL, 2 m²/mL bed volume to 15 m²/mL, 2 m²/mL bed volume to 10 m²/mL, 2 m²/mL bed volume to 9 m²/mL, 2 m²/mL bed volume to 8 m²/mL, 2 m²/mL bed volume to 7 m²/mL, 2 m²/mL bed volume to 6 m²/mL, 2 m²/mL bed volume to 5 m²/mL, 3 m²/mL bed volume to 20 m²/mL, 3 m²/mL bed volume to 15 m²/mL, 3 m²/mL bed volume to 10 m²/mL, 4 m²/mL bed volume to 20 m²/mL, 4 m²/mL bed volume to 15 m²/mL, 4 m²/mL bed volume to 10 m²/mL, 5 m²/mL bed volume to 20 m²/mL, 5 m²/mL bed volume to 15 m²/mL, or 5 m²/mL bed volume to 10 m²/mL when the support substrate has an average pore size of 0.1 μm to 10.0 μm, such as 0.2 μm to 0.5 μm.

Embodiment 35 is the separation media, separation device, or method of any one of embodiments 1 to 34, wherein the separation media has an average pore size of 10 micrometer (μm) or less, 5 μm or less, 2 μm or less, 1 μm or less, 0.6 μm or less, 0.5 μm or less, 0.45 μm or less, or 0.2 μm or less. The membrane may have an average pore size of 0.1 μm or greater, 0.2 μm or greater, 0.45 μm or greater, 0.5 μm or greater, 0.6 μm or greater, 0.7 μm or greater, or 1 μm or greater. The membrane may have an average pore size ranging from about 0.1 μm to 10.0 μm, 0.1 μm to 0.2 μm, 0.1 μm to 0.45 μm, 0.1 μm to 0.5 μm, 0.1 μm to 1 μm, 0.2 μm to 0.45, 0.2 μm to 0.50, 0.2 μm to 1 μm, 0.2 μm to 2 μm, 0.2 μm to 10 μm, 0.45 μm to 1 μm, 0.45 μm to 2 μm, 0.45 μm to 10 μm, 1 μm to 2 μm, or 1 μm to 5 μm. In some embodiments, the support substrate has an average pore size of 0.1 μm to 0.5 μm, 0.1 μm to 0.6 μm, 0.1 μm to 0.3 μm, or 0.4 μm to 0.6 μm.

### EXAMPLES

These examples are merely for illustrative purposes only and are not meant to be limiting on the scope of the appended claims. All parts, percentages, ratios, etc. in the examples and the rest of the specification are by weight, unless noted otherwise.

### Example 1: Assessment of Synthetic Methods to Prepare Separation Media

Two amine assisted method synthetic techniques were explored to install the support substrate reactive handles of the separation media on a base membrane. Without wishing to be bound by theory, it was thought that the incorporation of amine assistance groups may allow for a high density of the negatively charge fetuin or mucin affinity groups to be immobilized on the separation media.

In a first synthetic strategy, separation ligands were indirectly immobilized on a support substrate using an amine assisted method. Poly(2-hydroxyethyl acrylate) (poly(HEA)), poly(PEGMA-360), and poly(PEGMA-500) were grafted from a regenerated cellulose (pore sizes ranging from 0.2 μm to 3 μm) support substrate via surface-initiated activators generated by electron transfer atom transfer radical polymerization (SIAGET ATRP) activation process. Poly(HEA) was chosen as it is a hydrophilic polymer with low nonspecific binding.

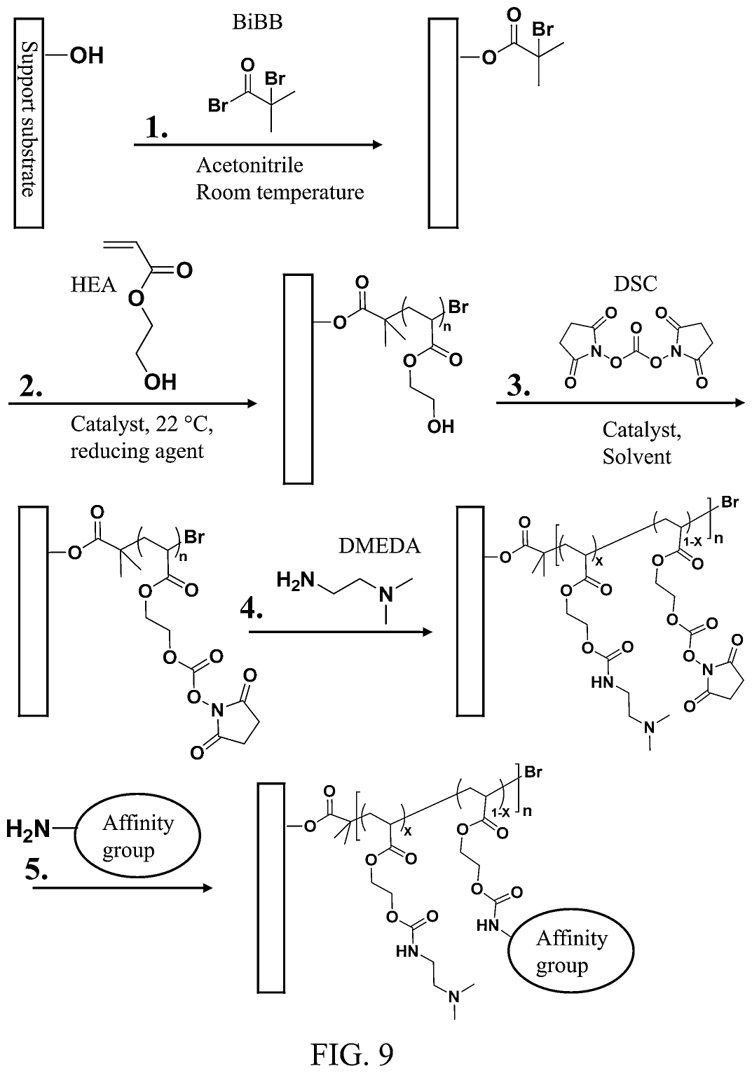

FIG. 9 shows the schematic of the synthetic strategy employed. In the first step, an initiator (α-bromoisobutyryl bromide, BiBB) was coupled to the OH groups of the regenerated cellulose (RC) membrane. In the second step, hydroxyethyl acrylate monomers were polymerized from the immobilized initiator to from poly(HEA). The next step (step 3) was to react the poly(HEA) reactive handles (OH groups) with the bifunctional (has two NHS ester reactive handles) linker precursor disuccinimidyl carbonate (DSC). The poly(HEA) reactive handles (OH) react with the first NHS ester of the linker precursor to form a first carbamate reaction product. In step 4, a portion of the second NHS ester groups are reacted with the primary amine reactive handle of N,N-dimethylethylenediamine (DMEDA) to form an amide reaction product and install the separation ligands containing the amine assistance groups. The DMEDA groups (e.g., the tertiary amine of DMEDA) act as cationic assisting groups to increase the local concentration of the negatively charged fetuin or mucin affinity groups (in aqueous buffer) at the membrane surface through coulombic interaction. In step 5, the second portion of the second NHS ester groups are reacted with the separation group (fetuin or mucin affinity group) precursor reactive handle to form an amide reaction product and install the separation ligands containing the affinity group on the support substrate. The membrane was exposed to a tris base solution as a final step to quench unreacted NHS intermediates and to install separation ligands containing a capping group. With the cationic assistance groups, a high density ligand immobilization using low ligand concentration was achieved, which is important to reduce production cost.

Although FIG. 9 shows the synthetic scheme for creating a separation membrane that includes poly(HEA), the same synthetic scheme can be used to make separation membranes with different polymers grafted from the RC membrane by simply substituting the HEA monomer in step 2 with different monomers. For example, separation media using the synthetic scheme in FIG. 9 were made by polymerizing a PEGMA-360 monomer (Mn of 360) and a PEGMA-500 monomer (Mn of 500 Da) to create separation media having poly(PEGMA-360) and poly(PEGMA-500).

Different initiator concentrations and reaction times were explored with the purpose of increasing the chain density while maintaining good ligand accessibility, low backpressure, and uniformity across larger membranes (e.g., support substrates). In general, lower initiator concentrations or shorter reaction times resulted in lower ligand density and lower binding capacity. High initiator concentrations or longer initiation times resulted in higher ligand degrees of grafting. It was found increasing initiator concentration was found to positively correlate with degree of grafting.

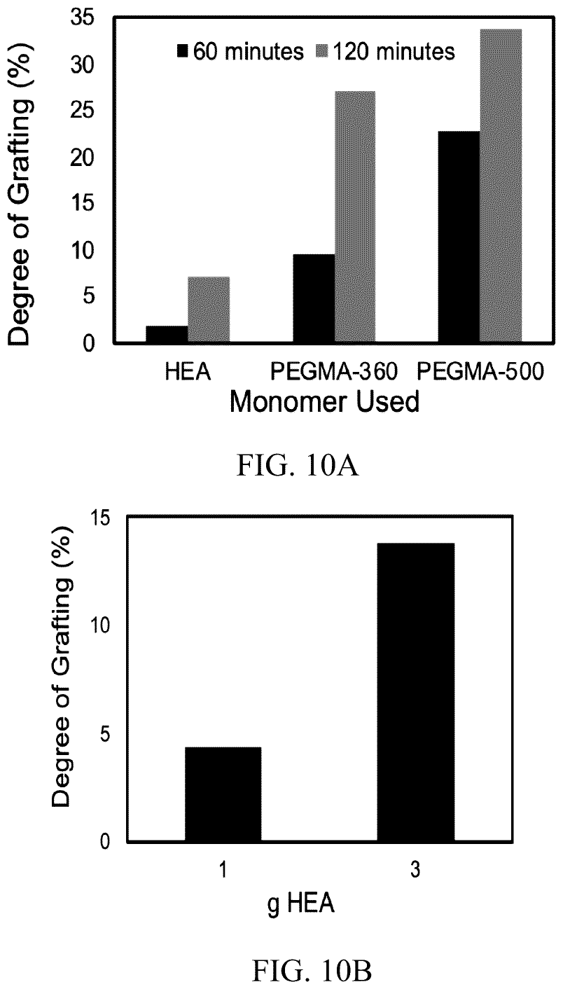

FIG. 10A shows the results of varying the initiation time (e.g., the amount of time the first step is allowed to proceed for). After initiation for 60 or 120 minutes, membranes were incubated in polymerization solutions of HEA monomers, or monomers to makePEGMA-360 or PEGMA-500 for 30 minutes. Increasing initiation time was found to positively correlate with degree of grafting with all polymers tested. As such, increasing the amounts of initiation time can be used to manipulate the grafting density of polymers.

FIG. 10B shows the results of varying the HEA monomer concentration (e.g., the amount of monomer added in step 2). After initiation for 60 minutes, membranes were incubated in polymerization solutions containing 1 gram (g) or 3 g HEA for 30 minutes. Increasing monomer amount in polymerization was found to positively correlate with degree of grafting.

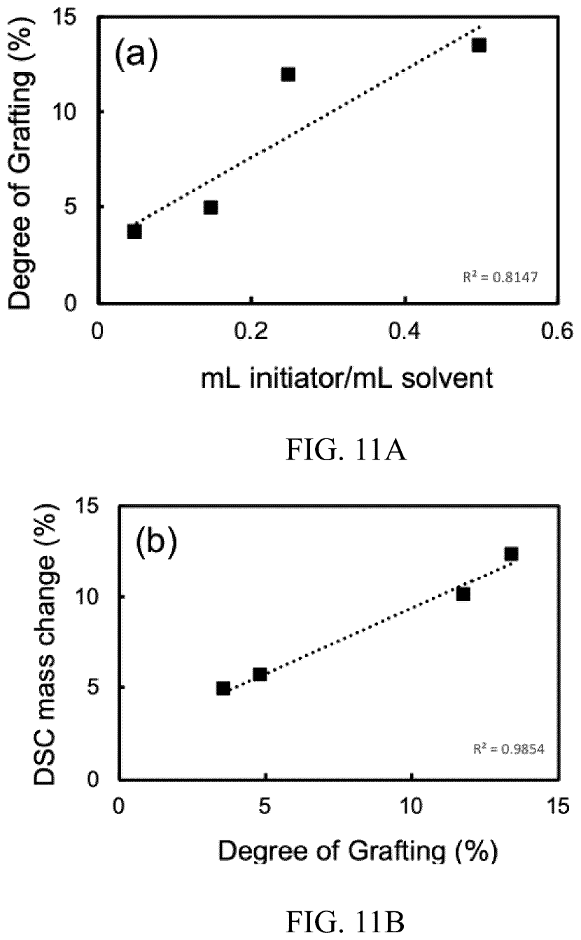

Shown in FIG. 11A, the initiator concentration was found to have a positive correlation with degree of grafting using 1 g HEA in polymerization solution. Shown in FIG. 11B, the degree of grafting was found to have a linear relationship with percent mass change following the coupling of DSC in step 3.

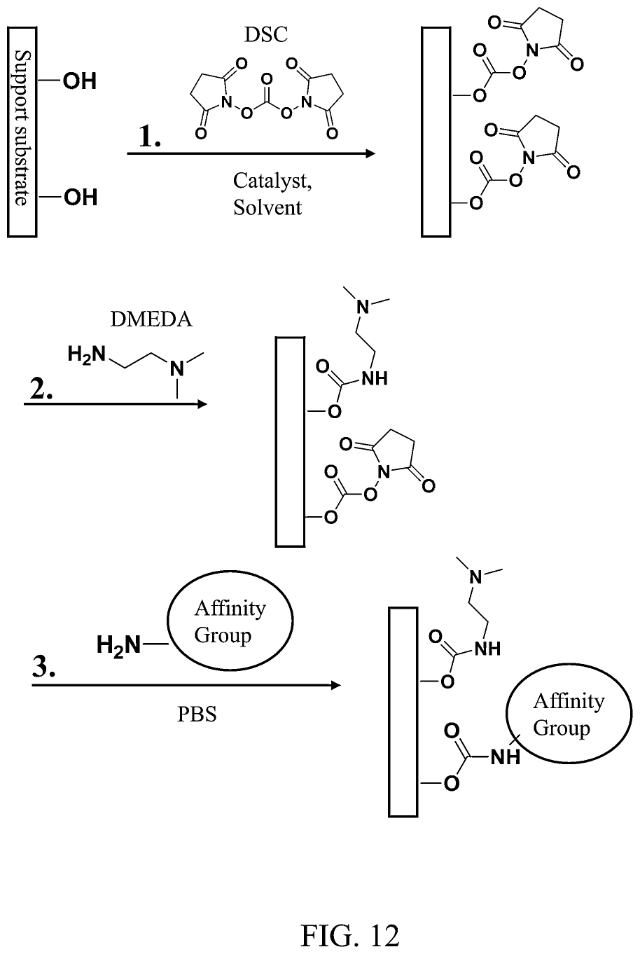

While ATRP polymerized substrates were found to be effective to increase activation density, it was shown that modulation of support substrate porosity was a more convenient method of increasing binding capacity. RC membranes were chosen for direct immobilization of the separation ligands using the amine assisted method for making separation media. FIG. 12 shows an amine assisted method used to construct some of the separation medias of Example 1.

The direct amine assisted amine method was similar to the polymer amine assisted method except that the membrane was not functionalized with a polymer. Instead, the hydroxyl reactive handles of the membrane were directly reacted with one of the NHS ester reactive handles of DSC to form a carbamate reaction product.

### Example 2: Additional Synthetic Method to Prepare Separation Media

Residual tertiary amine moieties in the final separation media may have the potential for nonspecific binding when the solution conductivity is very low. As affinity chromatography typically is performed at conductivity levels above that which tertiary amines retain significant binding capacity, the residual amine groups were expected to have negligible effect on chromatographic performance. In an effort to circumvent this potential issue completely, an organic solvent assisted coupling method was employed to install the separation ligands containing the affinity group without the use of amine containing assistance groups. The organic assisted coupling method utilizes water-miscible organic solvents as a constituent of the immobilization solution to enhance protein (carbohydrate containing protein affinity group) coupling efficiency, which enables use of low protein concentrations in the coupling solution. Additions of organic solvents to the aqueous buffered coupling solution (10%-80% by volume, dependent on the organic solvent used) to bring solution near the cloud point. At the cloud point, the protein solution starts to appear turbid upon increasing the concentration of organic solvent. Organic solutions replace water molecules in the protein's solvation shell which can facilitate greater interaction between the separation ligand and the membrane (support substrate). Additional organic solutions added beyond the cloud point exacerbate aggregation and flocculation dynamics of the ligand, which can comparatively reduce efficiency of coupling reaction. This coupling methodology allows for high performance membranes to be prepared with low affinity group consumption.

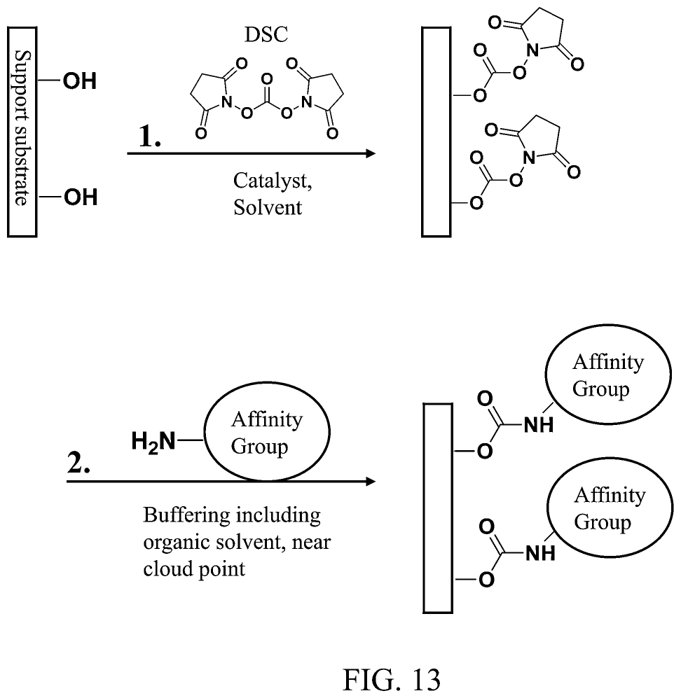

FIG. 13 shows an example synthetic scheme that may be used to prepare separation media of the present application via direct immobilization of the separation ligands using the organic solvent assisted coupling method. In step 1 the RC membrane reactive handles (OH) (the support substrate reactive handles) react with the first N-hydroxy succinimidyl (NHS) ester of the DSC linker precursor to form a carbamate reaction product. In the second step, a reaction mixture that is near the cloud point that includes the separation group precursor (includes the affinity group), water, and a water-miscible solvent is exposed to the reaction product of step 2. The second NHS ester groups of the support substrate-linker conjugate react with the reactive handle (NH₂) of the separation group precursor to form an amide reaction product and install the separation ligands having the affinity group on the support substrate.

### Example 3: Bind-and-Elute Condition Screen

Separation media prepared in Examples 1 and 2 were screened for binding capacity and the ability to bind and elute already pure active recombinant hemagglutinin (rHA) produced in E. coli. Static (e.g., equilibrium) binding capacity (SBC) and dynamic binding capacity at 10% breakthrough (DBC_{10%}) over a commercially relevant range of conditions were evaluated. The ability to elute rHA with high recovery while maintaining vaccine function is important for vaccine applicability. Separation media demonstrating high binding capacity were used to scout optimum buffer conditions for binding and eluting rHA with high recovery and activity.

E. coli-derived recombinant HA has traditionally lacked native HA characteristics for effective vaccine function. It is known that the HA1 subunit (eHA1) of H1N1 pandemic influenza virus produced in E. coli forms biologically active oligomers/trimers with native receptor binding activity and is highly immunogenic in animal models, comparable to egg- and baculo-derived HA.

Influenza HA binds to cells through the recognition of membrane bound proteins that include sialic acid (SA) groups. Influenza type and subtype can have specificities related to the a linkage of the SA, the majority tend to interact more strongly with α-2,6-linked SA over α-2,3-linked SA. Therefore, initial attempts focused on the immobilization of sialic acid rich proteins such as mucin and fetuin. Using the previously described coupling methodologies in Example 1 and Example 2, mucin and fetuin separation media were fabricated. An alternative approach to the immobilization of fetuin was taken with the understanding that immobilized boronates are able to interact with cis-diols (such as those in the sialyations in fetuin). It was ultimately determined that separation media having separation ligands that included a fetuin affinity group directly immobilized to the support substrate and prepared using the organic solvent coupling methodology (described in Example 2) were the best candidates to advance, with reduced binding associated with other methods. Additionally, there was little binding of HA found to be associated with mucin functionalization, regardless of the coupling methodology. Using membranes with optimized fetuin density, SBC performance was assessed using eHA1 in 20 mM phosphate at pH 7.3. The static binding capacity of the membrane was measured to be ˜10 mg eHA1/mL separation media. Scaled-down, high-throughput elution experiments were performed by loading eHA1 to separation media and applying various buffers in sequence and utilizing mass balance using UV spectroscopy to measure the eHA1 content of each eluate fraction. Additional bind and elute cycles were performed once elution was observed. The following parameters were tested:

### Binding Conditions

1. Low conductivity phosphate buffers pH ˜7.0 were identified as the suitable binding conditions.

### Elution Conditions

1 Water structuring based: 20 mM Sodium Phosphate with 1 M, 2 M, or 3 M sodium chloride at pH 7.0; 0.1 M boric acid, 0.1 M tris base, 0.1 M potassium chloride, 2 mM EDTA, 3 M potassium chloride at pH 8.2; 0.25 M histidine, 0.5 M arginine, at pH 7.5; 0.25 M histidine, 0.5 M arginine, at pH 9.86; 100 mM glycine at pH 3.0; 6 M urea, 0.55 mM triton x-100, 8.5 mM SDS.

2 Competitive: 0.4 M lactose; 0.4 M methyl glucoside; 0.4 M methyl mannoside; 0.4 M sorbitol; 0.4 M N-acetyl-D-glucosamine.

3 Combinatory: 0.1 M sialic acid, 1 M NaCl, 2 mM EDTA, 20 mM Tris at pH 7.4; 0.1 M methyl mannoside, 2.5 mM sodium phosphate at pH 3; 0.1 M methyl mannoside, 0.1 M N acetylglucosamine, 2.5 mM sodium phosphate at pH 3.0; 0.1 M methyl mannoside, 2.5 mM sodium phosphate at pH 2.0; 0.1 M methyl mannoside, 0.1 M N-acetylglucosamine, 2.5 mM sodium phosphate, at pH 2.0.

4. Control: 0.1 M NaOH

5. Other: proprietary elution buffer.

The eluents resulting in greatest amount recovered were 20 mM sodium phosphate with >1 M NaCl, 8.5 mM SDS, and the proprietary elution buffer, with little success found with any other eluant. For subsequent bind and elute testing; 20 mM sodium phosphate at pH 7.0 was selected for binding and the proprietary elution buffer was used in elution due to superior performance relative to 20 mM sodium phosphate with any amount of NaCl and to avoid UV aberration from micellar entrapment, aggregate formation, and protein denaturation associated with SDS. The best conditions identified by this study were used as a starting point to further optimize bind-elute conditions for purification of HA from cell supernatant in which purity and biological activity were measured in addition to binding capacity and recovery.

### Example 4: Assessment of Separation Media for Capture of Recombinant HA Protein Influenza Vaccine

Separation media having fetuin on the surface (made similar to FIG. 13) were assembled into columns with 0.1 mL bed volume and put into 2.5 cm syringe filter-like with a polypropylene housing using an ultrasonic welder. The columns had luer lock ports for simple “plug-and-play” connection to common chromatography systems (e.g., Fast Protein Liquid Chromatography).

Various recombinant HA proteins and whole HA-based vaccine were used to test the ability of the separation media (through testing the columns that included the separation media) of the present disclosure to capture and elute active protein and/or vaccines. The HA proteins tested included H1N1 A/California/07/2009 virus (eHA1); H3 Type A (rH3); B/Victoria rHA, B/Yamagata-like rHA. Additionally, type B whole influenza virus was also tested.

The columns were connected to a fast paced liquid chromatography (FPLC) system and equilibrated with buffer. The rHA1 feed solution was loaded onto the column, followed by rinse buffer to flush out unbound impurities. The vaccine product and bound impurities were released by an eluent. A strip solution (0.1 M NaOH) was applied to remove any remaining adsorbed species on the column. The flow-through, elution, and strip fractions were collected for analysis.

Specifically, using an FPLC at a flow rate of 0.75 mL/min (10 CV/min) an affinity column was equilibrated with 20 mM sodium phosphate at pH 7.3 and challenged with 2 mL of 1 mg/mL eHA1 that had previously undergone a propriety purification method. It was observed that some eHA1 flowed through the column and was not bound. It was suspected that flowthrough is due in part to inactive forms of eHA 1 but could also be resultant from membrane saturation. Nevertheless, using 20 mM sodium phosphate with 3 M NaCl as an elution buffer (note, this is not the proprietary elution buffer), a clear elution peak was observed prior to running the buffer UV absorbance shift due to transition to NaCl laden eluate. These early results indicated that the separation ligands including the fetuin affinity group was able to bind and elute eHA1.

Small volume purifications may be challenging using computer and pump aided chromatography systems due to the dead volume in the system. To reduce eHA1 loss due to system hold up (dead volume in the system), purifications were instead performed manually using a syringe to drive 5 mL of equilibration solution (20 mM sodium phosphate at pH 7.3) through the affinity column unit at a flow rate of 1 mL/min. Samples of 10 mL eHA1 that had already undergone the proprietary purification method in the same buffer were applied using the same methodology at 0.4 mL/min before eluting 20% of the initial protein load with 20 mM sodium phosphate at pH 7.3 with 2 M NaCl at 1 mL/min. eHA activity was determined to be equal in both the feed sample and the elution, with no active eHA found in the flowthrough. This suggests that of the total protein extracted from the proprietary purification method that only about 20% was found to be active and supports the notion that active eHA1 was selectively captured during the purification process and able to be eluted, resulting in enrichment in its active form per unit protein. This is a successful proof of concept indicating that affinity purification of rHA can improve active component per unit protein.

Sample absorbance at 280 was found to be sub optimal for enhanced analysis and lower wavelengths were attempted. It was found that optimal wavelengths were below 230 nm, outside of the range of the spectrophotometer used. After screening several wavelengths (200, 210, 214, 220, 240, and 280 nm), size exclusion chromatography (SEC) HPLC was utilized to identify eluate purity. Unfortunately, several elements in the proprietary elution buffer were found to overlap with impurities found in the feed, however, rHA in these reduced wavelengths was able to be isolated and retention time was identified to be around 18.3 minutes. SEC HPLC was run for the rHA feed (after proprietary purification but before exposure to the column of the present example); a sample after the rHA feed was exposed to the column and eluted with no column washing step; and after the rHA feed was exposed to the column, the column washed, and the rHA eluted. Analysis of the HPLC chromatograms shows that although modest, the percentage of rHA in the sample was able to be increased via purification using the column of the present example.

Starting from results of the bind-elute screening study in Example 3, the effect of those parameters (buffer type, pH, additives) as well as membrane synthesis conditions was further evaluated based on vaccine purity, recovery, and activity. Protein species in samples were resolved and approximate molecular masses determined using standard SDS-PAGE. The relative abundance of protein species was determined by comparing band intensity in SDS-PAGE gels. HA protein concentrations before and after separation by the column and was determined by UV spectroscopy. The activity of the recombinant HA and virus containing HA was assessed by a hemagglutination assay using 0.5% turkey red blood cells and was expressed as hemagglutinin score.

Additional bind-and-elution conditions were evaluated and tested for yield against the proprietary purification (PP) method used in the pre-purification of the samples discussed in the above experiments of this Example. In subsequent rounds of testing, additional improvements were made to the initial elution conditions (elution method 1) which resulted in improved yields (elution method 2; improvements by up to 100% in some cases). It was found that for H1 rHA both the amount recovered, and activity were higher than the PP method. However, for H3 rHA despite there being improved yields, there was little difference in activity compared to proprietary eluates (Table 4).

TABLE 4
Comparison of separation media (membranes) of the present
disclosure and a proprietary purification method

| | Minimum | | | | |
|---|---|---|---|---|---|
| | Binding | Elution Method 1 | | Elution Method 2 | |
| | Capacity | Yield (mg | % | Yield (mg | |
| | (mg rHA/mL | rHA/mL | Yield | rHA/mL | % Yield |
| rHA Type | membrane) | membrane) | vs PP | membrane) | vs PP |
| Victoria B | 13.2 | 4 | 29 | 4 | Not tested |
| Yamagata- | 5.4 | 2 | 37 | 2 | 107 |
| like B | | | | | |
| Yamagata- | 5.2 | 2 | 42 | 2.2 | 81 |
| like B | | | | | |
| repeat | | | | | |
| rH3 | 6.36 | 2 | 28 | 1.8 | 81 |

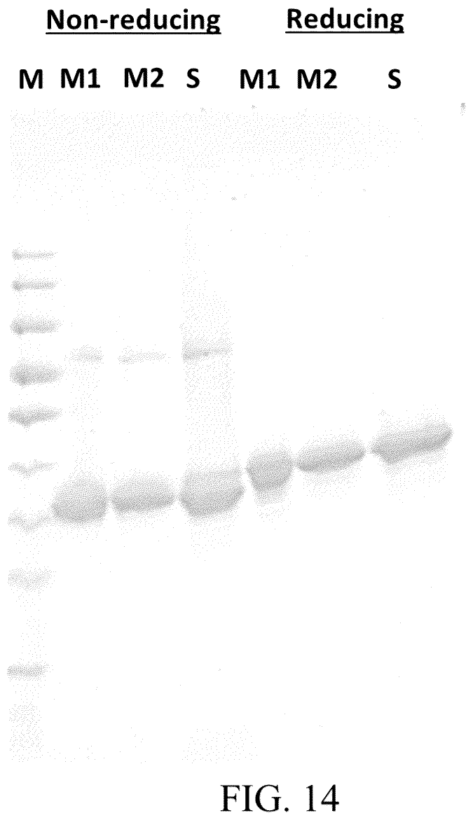

To benchmark against existing technology a comparative experiment was performed to compare the columns of the present disclosure and a Cytiva CAPTO S IMPACT (Cytiva Life Sciences, Marlborough, MA) column following previously optimized elution protocols. Both non-reducing and reducing conditions were applied to eluates from the columns of the present disclosure using both elution methods 1 (M1) and 2 (M2) as well as elution from a Cyvita CAPTO S IMPACT purification. FIG. 14 shows an SDS PAGE gel of purified rHA samples using the affinity columns (separation media) of the present disclosure and Cytiva CAPTO S IMPACT columns. Results indicate that the columns of the present disclosure showed fewer impurities in the elution pool as indicated by reduced smearing in non-reducing conditions and fewer bands present in reducing conditions than the Cytiva CAPTO S IMPACT columns.

Once feasibility was demonstrated using E. coli-derived HA, binding of whole type B influenza was confirmed in a similar fashion. These results along with binding of a plethora of rHA types in the comparison study suggest that the separation media has general HA specificity and indicates that purifications of HA produced in baculovirus and the traditional chicken egg platforms are likely to also be feasible. This broadened applicability would have greater commercial impact as it could provide an alternative to currently employed centrifugation techniques.

### Example 5. Assessment of Carbohydrate Affinity Groups That do not Include a Protein Component

Several approaches were attempted, with the goal of creating a fully synthetic sialic acid (Nue5Ac) rich separation media. Initial attempts focused on the in-house synthesis and coupling of modified 6′sialyllalactose with latter attempts focused on membrane modification with either α-2,3-linked and α-2,6-linked sialic acids exclusively. Separation media prepared with immobilized sialic acids with specific linkages may provide additional process flexibility to purification train.

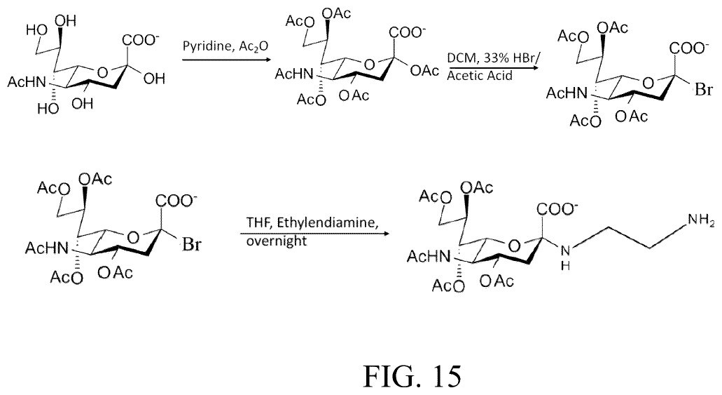

FIG. 15 shows the synthetic scheme used to synthesize 6′sialyllactose from Neu5Ac. Starting with 6′ sialyllactose, alcohol groups were protected using acetic anhydride catalyzed by pyridine. The crude product was then dried in dichloromethane (DCM) with sodium sulfate before subsequent removal of the salt by filtration. The product was then selectively brominated using 33% hydrobromic acid/acetic acid in DCM. Subsequent amination at the brominated site was performed via diamine conjugation in tetrahydrofuran (THF). The product was purified and immobilized to the support substrate using the synthetic scheme in FIG. 9. Immobilization may also be accomplished using the synthetic scheme in FIG. 12 or FIG. 13. Following coupling to the membrane, the alcohol groups of the sugars were then deprotected using either 0.01 or 0.1 M sodium hydroxide to restore the active form of the sugar.

Due to the low UV absorbance of rHA, coupled ligand density was determined using lectins such as wheat germ agglutinin (WGA). WGA has illustrated interaction with sialic acid residues in literature. Separation media examples were equilibrated by immersion in 20 mM Tris at pH 7.4 with 1 mM each of CaCl₂, MnCl₂, and MgCl₂ (to satisfy WGA's cofactor requirements) before being transferred to a 1 mg/mL solution of WGA in the same buffer. There was an increase in WGA bound associated with the strength of deprotection agent used indicating that sugar had been successfully coupled and restored to its functional condition (Table 5).

TABLE 5
Amount of WGA bound to separation media

| Membrane | SBC (mg WGA/mL membrane) |
|---|---|
| Control | 3.3 |
| 6′ sialyllactose with | 13.2 |
| 0.01M NaOH deprotection | |
| 6′ sialyllactose with | 19.5 |
| 0.1M NaOH deprotection | |

Encouraged by these results, further investigations into the immobilization synthetic sialic acids were performed. To remove carbohydrate synthesis yield and purity as confounding factors, asparagine coupled to glycans containing a α-2,3-linked or a α-2,6- linked sialic acid (Glyctech, Inc in Kyoto, Japan) were coupled to a support substrate using the organic solvent assistance method. For further evaluation, genetically modified enzymes with α-2,3-linked or α-2,6-linked specificity (available from LECTENZ BIO in Athens, GA; SIAFIND 2,3 and SIAFIND 2,6) were used in lieu of WGA to determine successful incorporation of synthetic sugars without the need for additional cofactors. Several immobilization conditions (method 1 and method 2) were evaluated for both carbohydrates and compared to an unmodified control and membranes prepared previously with optimized fetuin density (Table 6). It was shown that high densities of α-2,3- linked and α-2,6-linked sialic acid (SA) were able to be immobilized to the support substrate (notably in a range comparable to fetuin immobilized separation media). Additionally, membranes prepared with synthetic carbohydrates exhibited only carbohydrate/lectin specific interactions. As such, in contrast to fetuin which contains both α-2,3-linked and α-2,6-linked sialic acids, separation media prepared with synthetic carbohydrates may be able to discriminate HA preferentially.

TABLE 6
SBC of various synthetic carbohydrate affinity
groups and synthetic methods used to prepare
the separation membranes containing the same

| Separation | | |
|---|---|---|
| media affinity | SBC of mg binding lectin per mL separation media | |
| group | SIAFIND 2,3 | SIAFIND 2,6 |
| Control | <LOD | 1.5 |
| Fetuin | 15.9 | 17.9 |
| 2,6 Set 1 Method 1 | <LOD | 13.9 |
| 2,6 Set 1 Method 2 | <LOD | 23.3 |
| 2,6 Set 2 Method 2 | <LOD | |
| 2,3 Set 1 Method 1 | 13.5 | |
| 2,3 Set 1 Method 2 | 18.5 | |
| 2,3 Set 2 Method 2 | 15.0 | 2.1 |

LOD is limit of detection.

The complete disclosure of all patents, patent applications, and publications, and electronically available material cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

1.-34. (canceled)

35. A separation media comprising:
a support substrate; and
a plurality of separation ligands for formula SL1 or SL2 [symbol]
Rp¹-z (SL1) [symbol]
Rp³-Sp-Rp⁴-Z (SL2)
wherein:
Z is a separation group comprising an affinity group, the affinity group comprising a carbohydrate, a small molecule, or both; and
Rp¹, Rp³, and Rp⁴ each independently comprise the reaction product of any one of Rp^{A}, Rp^{B}, Rp^{C}, Rp^{D}, Rp^{E}, Rp^{F}, Rp^{G}, Rp^{H}, Rp^{I}, Rp^{J}, Rp^{K}, or an isomer thereof, wherein Rp^{A}, Rp^{B}, Rp^{C}, Rp^{D}, Rp^{E}, Rp^{F}, Rp^{G}, Rp^{H}, Rp^{I}, Rp^{J}, Rp^{K} are represented by: 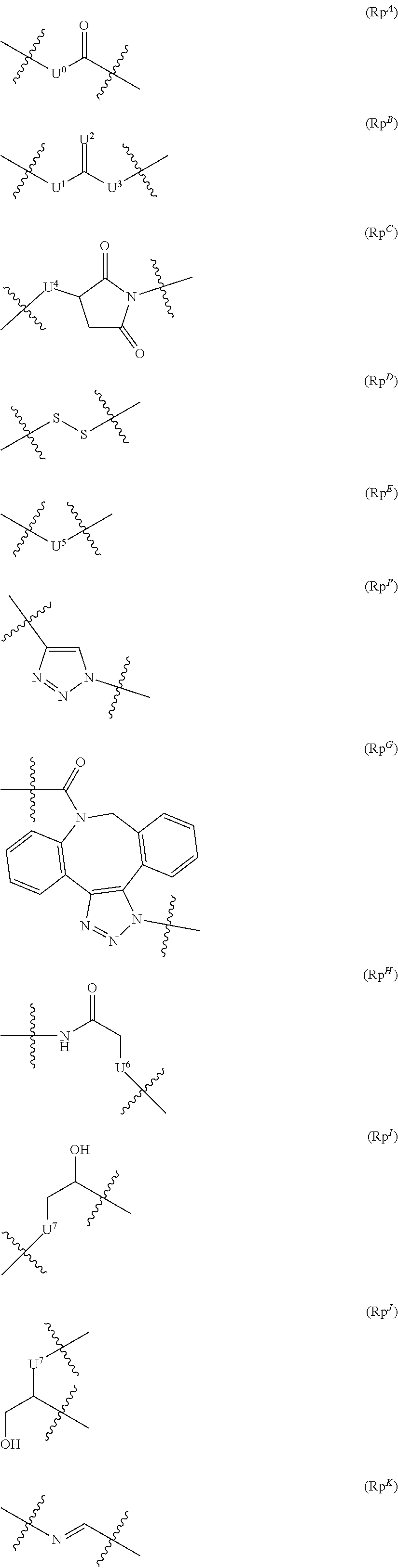
wherein:
U⁰, U¹, U², U³, U⁴, US, U⁶, and U⁷ are each independently NH, O, or S;
Sp is a spacer comprising a divalent organic group, and
wherein the separation media is configured for isolating a target molecule comprising a carbohydrate binding domain.

36. The separation media of claim 35, wherein the alkanediyl or alkenediyl comprises a backbone chain of length C1 to C3.

37. The separation media of claim 35, wherein Sp comprises —C(O)—.

38. The separation media of claim 35, wherein Rp³ and Rp⁴ comprises Rp^{E}.

39. The separation media of claim 38, wherein each U⁵ is O, each U⁵ is NH, or one U⁵ is NH and U⁵ is O

40. The separation media of claim 35, wherein SL2 comprises 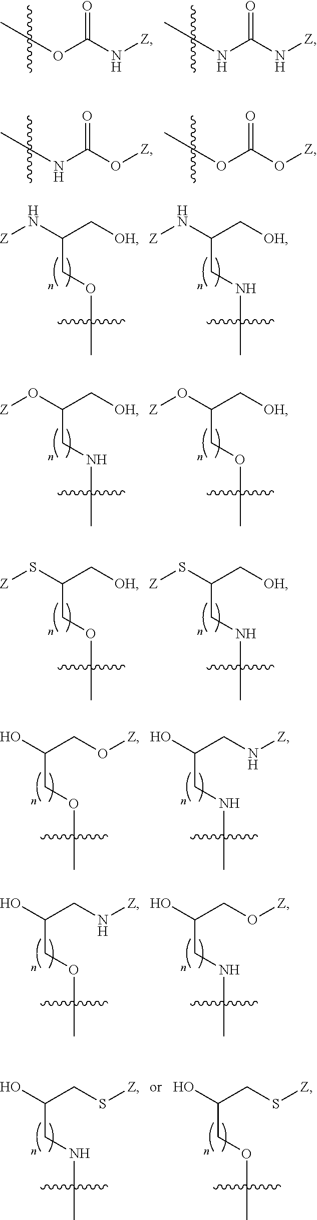
wherein n is 0, 1, 2, 3, 4, 5, 6, 7, 8, 9, or 10.

41. The separation media of claim 35, wherein the separation ligand formula SL or SL1 is of formula X, XI, XII, or XIII: 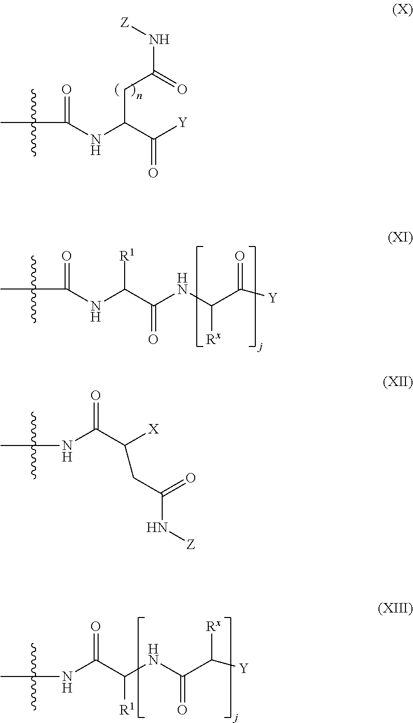
wherein n is 0, 1, 2, 3, or 4;
wherein X in the formula is NH₂ or PG_{N} where PG_{N} is an amine protecting group;
wherein Y is Y is OH or a PG_{C(O)OH} where PG_{C(O)OH} is a carboxylic acid protecting group;
wherein R¹ is an amino acid side chain;
wherein j is 1, 2, 3, 4, 5, 6, 7, 8, 9, or 10;
wherein each R^{X} is independently an amino acid side chain or 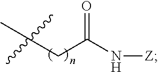
and wherein at least one R^{X} is

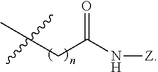

42. The separation media of claim 35, wherein the support substrate comprises a polyolefin membrane, a polyethersulfone membrane, a poly(tetrafluoroethylene) membrane, a nylon membrane, a fiberglass membrane, a hydrogel membrane, a hydrogel monolith, a polyvinyl alcohol membrane, a cellulose membrane, a cellulose ester membrane, a cellulose acetate membrane, a regenerated cellulose membrane, a cellulosic nanofiber membrane, a cellulosic monolith, a filter paper, or any combination thereof.

43. The separation media of claim 35, wherein the carbohydrate comprises a glycan. 44 The separation media of claim 43, wherein the glycan has a linear structure.

45. The separation media of claim 43, wherein the glycan has a branched structure with two terminal ends.

46. The separation media of claim 44, wherein the glycan comprises a terminal sialic acid and an adjacent saccharide, and wherein the terminal sialic acid and the adjacent saccharide are covalently linked through a linkage.

47. The separation media of claim 46, wherein the linkage is an α-2,6 linkage or α-2,3 linkage.

48. The separation media of claim 35, wherein carbohydrate binding domain comprises hemagglutinin.

49. The separation media of claim 35, wherein the target molecule comprises a recombinant hemagglutinin.

50. A separation device comprising a housing and the separation media of claim 35 disposed within the housing.

51. A method of isolating a target molecule from an isolation solution:
the isolation solution comprising:
an isolation solvent;
the target molecule comprising a carbohydrate recognizing domain;
the method comprising:
contacting the isolation solution with the separation media of claim 35.

52. The method of claim 51, wherein the method further comprises washing the separation media with a washing solution.

53. The method of claim 51, wherein the method further comprises eluting the target molecule from the separation media.

54. The method of any one of claims 51, wherein carbohydrate binding domain comprises hemagglutinin.
